(19) United States
(12) Patent Application Publication
(10) Pub. No.: US 2011/0038892 A1
(43) Pub. Date: Feb. 17, 2011

(54) COMPLEXING SYSTEM

(75) Inventors: Bazbek DAVLETOV, Cambridge (GB); Frederic DARIOS, Cambridge (GB); Enrico FERRARI, Cambridge (GB); Mikhail SOLOVIEV, Surrey (GB)

Correspondence Address: BANNER & WITCOFF, LTD., 1100 13th STREET, N.W., SUITE 1200, WASHINGTON, DC, 20005-4051, US

(73) Assignees: MEDICAL RESEARCH COUNCIL, London (GB); ROYAL HOLOLOWAY AND BEDFORD NEW COLLEGE, Surrey (GB)

(21) Appl. No.: 12/854,514

(22) Filed: Aug. 11, 2010

Related U.S. Application Data
(60) Provisional application No. 61/319,204, filed on Mar. 30, 2010.
(60) Provisional application No. 61/233,286, filed on Aug. 12, 2009.

(51) Int. Cl. A61K 39/08; C07K 14/00; C07K 14/33; C40B 60/12; G01N 30/00; C12M 1/34; C07K 1/00

(52) U.S. Cl. 424/239.1; 530/350; 506/39; 422/69; 435/287.1; 530/402

(57) ABSTRACT

The invention relates to a complexing system comprising two polypeptide helices derived from a SNAP protein; one polypeptide helix derived from syntaxin; one polypeptide helix derived from synaptobrevin or a homolog thereof; and one or more cargo moieties attached to the polypeptide helices, wherein the four polypeptide helices can form a stable SNARE complex. The invention also relates to a method of producing the complexing system and the use of the complexing system.

This application contains and incorporates the electronic file containing the sequence listing which is 98 kb and is named “seqlst00104” and was created Aug. 11, 2010.

### FIELD OF INVENTION

The present invention relates to a complexing system for forming a molecular scaffold which is based on the SNARE complex.

### BACKGROUND TO INVENTION

In eukaryotic cells, vesicles are merged with a membrane in a process known as vesicle fusion. For example, vesicles can be merged with the cell plasma membrane or other cell compartments such as endosomes or lysosomes. The most studied form of vesicle fusion is the docking of synaptic vesicles with the pre-synaptic membrane in neuronal cells to release neurotransmitters to cause propagation of a nerve impulse in the post-synaptic neuron.

The process of vesicle fusion is mediated by SNARE proteins (SNAREs), which are a large protein superfamily consisting of more than 60 members in yeast and mammalian cells. SNAREs are small, abundant and mostly membrane-bound proteins. Although they vary considerably in structure and size, they all share segments in their cytosolic domains called SNARE motifs that are capable of assembly into a tight, four-helix bundle called a SNARE complex. It is thought that SNARE motifs are about 60-70 amino acids in length (Jahn R and Scheller R H), although this is not well defined. SNARE complexes are sometimes composed of three proteins: syntaxin and SNAP-25, which are resident in the cell membrane; and synaptobrevin (also referred to as vesicle-associated membrane protein or VAMP), which is anchored in the vesicular membrane.

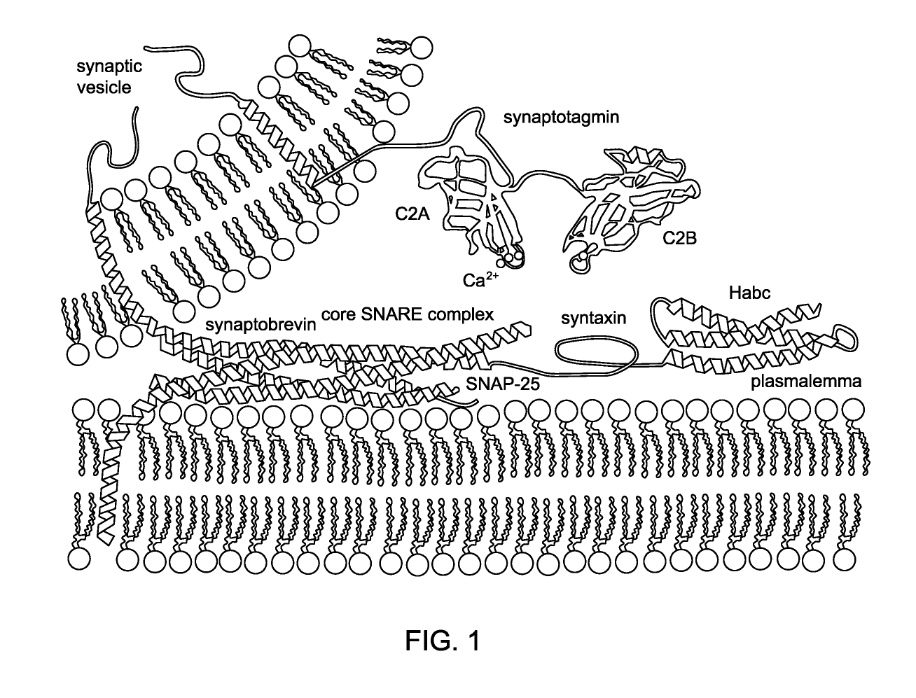

In neuronal exocytosis, syntaxin and synaptobrevin are anchored in their respective membranes by their C-terminal domains, whereas SNAP-25 is tethered to the plasma membrane via several cysteine-linked palmitoyl chains. The core SNARE complex is a four-α-helix bundle in which one α-helix is contributed by syntaxin, one α-helix is contributed by synaptobrevin and two α- helices are contributed by SNAP-25. This SNARE complex has been found to be very stable. A schematic representation of the molecular machinery involved in vesicle fusion is shown in FIG. 1.

### SUMMARY OF INVENTION

The inventors have found that the SNARE complex and the formation of it can be used for various applications. Therefore, the basis of the invention is a complexing system comprising:

two polypeptide helices derived from a SNAP protein;

one polypeptide helix derived from syntaxin;

one polypeptide helix derived from synaptobrevin or a homolog thereof; and

one or more cargo moieties attached to the polypeptide helices,

wherein the four polypeptide helices can form a stable SNARE complex.

In various aspects of the invention, further limitations can apply to the above complexing system. The invention also relates to a method of producing a stable SNARE complex using the above complexing system and use of the above complexing system and resulting stable SNARE complex.

### DETAILED DESCRIPTION OF INVENTION

The following description sets out a number of different embodiments of the invention. However, it will be apparent to one skilled in the art that virtually all the limitations, preferred features and variations described are applicable to other embodiments of the invention as long as they are all based on the complexing system described above.

In one aspect of the invention, there is provided a complexing system for forming a molecular scaffold, the system comprising:

two polypeptide helices derived from a SNAP protein;

one polypeptide helix derived from syntaxin;

one polypeptide helix derived from synaptobrevin or a homolog thereof; and

one or more cargo moieties attached to the polypeptide helices,

wherein the four polypeptide helices can form a stable SNARE complex, and wherein at least two of the polypeptide helices are less than 50 amino acids in length.

In other aspects of the invention, the complexing system may not have at least two of the polypeptide helices being less than 50 amino acids in length.

The present invention allows the controlled assembly of a stable complex formed of distinct functional units. The advantage of having a stable complex is that it can be used in relatively harsh conditions without the risk of the complex dissociating. This means that the helix or helices to which the one or more cargo moiety is/are attached will remain part of the complex, ensuring that the one or more cargo moiety does not dissociate from the rest of the complex.

As indicated above, the complexing system is based on the formation of a stable SNARE complex. Therefore, the four polypeptide helices of the present invention can, to a certain degree, have any sequence as long as they can form a stable SNARE complex.

In neurons, the SNARE complex is formed from the following proteins: SNAP-25; syntaxin; and synaptobrevin. These proteins, as well as other SNARE proteins, contain SNARE motifs or SNARE domains which are the portions of the proteins which are involved in forming the SNARE complex. These SNARE domains or motifs are helices which pack together to form the SNARE complex. Generally, only a portion of the SNARE proteins is involved in SNARE complex formation; not the entire SNARE protein. For example, syntaxin has a C-terminal trans-membrane domain, a SNARE domain and an N-terminal regulatory domain, also known as the head domain. Obviously, only the SNARE domain is involved in forming the SNARE complex.

The terms “SNARE motif” and “SNARE domain” are well known to those skilled in the art. Further, the SNARE motifs and SNARE domains of the various different SNARE proteins are also well known to a skilled person (Jahn R and Scheller R H (2006); Sieber et al. (2006); Besteiro (2006)).

The four polypeptide helices of the present invention are based on the SNARE domains or motifs of the SNARE proteins that form the SNARE complex, i.e. a SNAP protein; syntaxin; and synaptobrevin or a homolog thereof. Therefore, in one embodiment, the complexing system comprises: two polypeptide helices derived from the SNARE motif of a SNAP protein; one polypeptide helix derived from the SNARE motif of syntaxin; and one polypeptide helix derived from the SNARE motif of synaptobrevin or a homolog thereof, wherein the four polypeptide helices can form a stable SNARE complex. It may not be necessary for a helix of the invention to be the same length as the SNARE motif or domain from a SNARE protein. It may be shorter in length as long as it can still form a stable SNARE complex when complexed with the other helices of the invention.

The SNAP protein from which the two polypeptide helices are derived can be any SNAP protein which can form part of a SNARE complex. The skilled person is aware of the various SNAP proteins which can form part of a SNARE complex. For example, the SNAP protein may be SNAP-25A, SNAP-25B, SNAP-23 (also known as syndet), or SNAP-29. Preferably, the SNAP protein is not α-SNAP. Preferably, the SNAP protein is SNAP-25, i.e. SNAP-25A or SNAP-25B. SNAP proteins contain two SNARE motifs. Therefore, the two polypeptide helices may be derived from the two SNARE motifs in a particular SNAP protein. Alternatively, the two polypeptide helices may be derived from different SNAP proteins. Preferably, they are derived from the same SNAP protein.

The syntaxin protein from which the polypeptide helix is derived can be any syntaxin protein which can form part of a SNARE complex. The skilled person is aware of the various syntaxin proteins which can form part of a SNARE complex. For example, the syntaxin protein may be selected from syntaxin 1A, syntaxin 1B, syntaxin 2 (also known as epimorphin), syntaxin 3 and syntaxin 4, syntaxin 5, syntaxin 6, syntaxin 7, syntaxin 8, syntaxin 10, syntaxin 11, syntaxin 13, syntaxin 17 or syntaxin 18. Preferably, the syntaxin protein is syntaxin 1A or 3.

The synaptobrevin protein or homolog thereof from which the polypeptide helix is derived can be any synaptobrevin protein or homolog which can form part of a SNARE complex. The skilled person is aware of the various synaptobrevin proteins and homologs which can form part of a SNARE complex. Synaptobrevin is a member of the vesicle-associated membrane protein (VAMP) family. Other VAMP proteins are known to be able to form SNARE complexes and, therefore, may be suitable for providing the basis upon which a polypeptide helix can be derived. In one embodiment, homologs of synaptobrevin are VAMP proteins which can form part of a SNARE complex. Such VAMP proteins are well known to those skilled in the art. The synaptobrevin protein or homolog thereof may be selected from synaptobrevin 1, synaptobrevin 2, synaptobrevin 3 (also known as cellubrevin) and synaptobrevin 7 (also known as TI-VAMP). Preferably, the polypeptide helix is derived from a synaptobrevin protein. Preferably, the synaptobrevin protein is synaptobrevin 1, 2 or 3.

The organism from which the SNARE proteins originate can be any suitable organism in which SNARE complexes are utilised. For example, the proteins may originate from: mammals, such as humans, primates, and rodents; fish; and invertebrates, such as flies. Optionally, the SNARE proteins may be derived from yeast (Rossi G et al. (1997)). The organism from which the SNARE proteins originate may depend on the application of the complexing system. For example, for medical applications, the SNARE proteins preferably originate from humans.

The four polypeptide helices of the invention are derived from the SNARE proteins which form the SNARE complex. The helices of the SNARE motif or domain of the SNARE proteins which form the SNARE complex are generally about 60-70 amino acids in length. As indicated above, the four polypeptide helices of the invention are derived from the SNARE motif or SNARE domain of the SNARE proteins. The term “derived from” means that the sequence of the polypeptide helix is substantially the same as the sequence of the SNARE domain/motif or a portion thereof so that it is capable of forming a stable SNARE complex. Preferably, the sequence of the polypeptide helix should have at least about 80% sequence identity with the sequence of the selected SNARE domain/motif or the portion thereof. More preferably, the sequence identity should be at least about 85%, and even more preferably at least about 90%. In one embodiment, the sequence identity may be at least about 95%, at least about 98% or even 100%. However, in some embodiments, it may be preferable for the sequence of the polypeptide helix to differ from the sequence of the selected SNARE domain/motif or the portion thereof. This may be beneficial in terms of expression of the protein, purification of the protein or down-stream applications. For example, and without limitation, this may include the addition of histidine residues at either end of the sequence to enable purification, or incorporation of additional lysine or cysteine residues for functional attachment of the peptides to surfaces or cargoes.

The two SNAP derived helices may comprise a sequence selected from SEQ ID NOs. 1, 2, 5, 6, 24, 41, 48, 49, 50, 51, 64 and 65.

The syntaxin derived helix may comprise a sequence selected from SEQ ID NOs. 3, 7, 9, 25, 32, 33, 34, 35, 36, 37, 38, 46, 47, 60, 61, 62 and 66.

The helix derived from synaptobrevin or a homolog thereof may comprise a sequence selected from SEQ ID NOs. 4, 8, 27, 28, 29, 30, 31, 42, 43, 44, 45, 52, 53, 67, 68 and 69.

Further, the helices derived from the SNARE proteins may consist of the above sequences.

Additionally, a helix of the invention may comprise or consist of a sequence selected from SEQ ID NOs. 1-9, 12-15 and 18-69.

Each polypeptide helix should be long enough so that it can interact with the other helices to form a stable SNARE complex. In effect, this means that the polypeptide helix must be derived from a sufficiently long portion of the SNARE motif/domain to allow the formation of a stable SNARE complex. It is relatively straight forward and well within the capabilities of a skilled person to test whether a polypeptide helix is long enough to allow formation of a stable SNARE complex. This can be done by complexing the polypeptide helix with the other three polypeptide helices and testing to see whether the resulting SNARE complex dissociates under adverse conditions. Adverse conditions are those which generally cause dissociation of protein complexes and protein-protein interactions. Such conditions will be apparent to a skilled person. For example, such adverse conditions may be exposing the complex to a strong surfactant or a disrupting detergent. In one embodiment, a SNARE complex can be tested to determine whether it is stable by using SDS-PAGE which is performed in the presence of denaturing SDS concentrations (>0.02%). If the complex is dissociated by the SDS in the gel so that it separates into its component parts, the SNARE complex may not be stable. However, if the SNARE complex does not dissociate but remains as a single entity when moving through the SDS-PAGE gel and can be detected as such, for example, using Coomassie staining, it can be considered to be stable. Therefore, in one embodiment, the SNARE complex can be considered to be stable if it does not dissociate using SDS. Preferably, the SNARE complex is stable when using a gel sample buffer for SDS-PAGE containing about 2% SDS. More preferably, the SNARE complex is stable when using a gel for SDS-PAGE containing about 0.1% SDS. This can be done using a gel for the SDS-PAGE which contains about 0.1% SDS.

Alternatively, the stability of the SNARE complex can be verified by bead pull-down. The SNARE complex can be considered to be stable if one component of the complexing system will bring down all interacting partners in a stoichiometric manner. Bead pull-down is well-known to a skilled person (Rickman C. et al. (2004)).

Surface plasmon resonance measurement of protein-protein interaction can also be used and is well known to those skilled in the art (Karlsson & Falt (1997)). When using surface plasmon resonance, the SNARE complex can be considered to be stable if one does not observe dissociation evidenced by the decrease of the surface plasmon resonance signal.

Another method of determining whether a SNARE complex is stable is to determine the dissociation constant of the complex. This is the dissociation constant for one helix dissociating from the complex. A stable SNARE complex should have a dissociation constant of less than 10⁻⁷ M. In certain embodiments the stable SNARE complex may have a dissociation constant of less than 10⁻⁸, 10⁻⁹, 10⁻¹⁰, 10⁻¹¹, 10⁻¹², 10⁻¹³, 10⁻¹⁴, or 10⁻¹⁵ M. Depending on the application that the complexing system is used for, it might be beneficial to have different dissociation constants. Therefore, in some embodiments, the dissociation constant may be 10⁻⁷ M to 10⁻¹¹ M or 10⁻⁷ M to 10⁻¹⁰ M. Alternatively, the dissociation constant may be comparable to antibody dissociation constants (6-10×10⁻⁸ M).

SNARE complex formation can also be assessed by binding to complexin which binds to the fully formed SNARE complex (Hu et al. (2002)).

In one embodiment, the polypeptide helices of the present invention are at least about 25 amino acids in length. SNARE complexes using such helices can form in solution. However, they may not be SDS resistant. Preferably, the polypeptide helices of the present invention are at least about 30 or about 35 amino acids in length. More preferably, the polypeptide helices of the present invention are at least about 40 amino acids in length. SNARE complexes formed using such helices are generally SDS resistant. Alternatively, the polypeptide helices may be at least about 45 amino acids in length.

In one embodiment, at least one of the polypeptide helices is less than 50 amino acids in length. The advantage of this is that it is much easier to artificially manufacture polypeptides which are less than 50 amino acids in length. The polypeptide helices which are less than 50 amino acids can be any of the polypeptide helices. Preferably, at least two of the polypeptide helices are less than 50 amino acids in length. Preferably, they are the polypeptide helices derived from syntaxin and synaptobrevin or a homolog thereof.

In alternative embodiments, three or four of the polypeptide helices may be less than 50 amino acids in length. In such embodiments, any combination of helices can be less than 50 amino acids in length. The inventors surprisingly found that minimal peptide sequences of the SNARE motif of each SNARE protein are sufficient to form a high-affinity stable four helical bundle SNARE complex. This is advantageous for ease of production of the peptides. Furthermore, each minimal sequence can carry a cargo, which retains functionality.

Where two of the polypeptide helices are less than 50 amino acids in length, preferably it is the helix derived from syntaxin and the helix derived from synaptobrevin or a homolog thereof which are less than 50 amino acids in length.

The four polypeptide helices of the invention may be about the same length or may be different in length. For example, in one embodiment they may all be about 45 amino acids in length. Alternatively, the polypeptide helices may be different lengths.

In one embodiment of the invention, the polypeptide derived from syntaxin and/or the polypeptide derived from synaptobrevin or a homolog thereof may further comprise a membrane attachment sequence to allow immobilisation of the SNARE complex to membranes or lipid bilayers. Such membrane attachment sequences are well known to those skilled in the art. For example, the native sequences of syntaxin and synaptobrevin contain transmembrane portions to allow attachment of the protein to vesicle and cellular membranes (Kasai and Akagawa (2001) and Laage et al. (2000)). Therefore, in one embodiment, the polypeptide helix derived from syntaxin may further comprise a membrane attachment sequence derived from the syntaxin transmembrane domain for immobilising the polypeptide helix to a membrane or lipid bilayer. Similarly, the polypeptide helix derived from synaptobrevin or a homolog thereof may further comprise a membrane attachment sequence derived from the transmembrane domain of synaptobrevin or a homolog thereof for immobilising the polypeptide helix to a membrane or lipid bilayer.

A stable SNARE complex is one which does not dissociate under adverse conditions which generally cause dissociation of protein complexes and protein-protein interactions. This may be measured, for example, using SDS-PAGE, bead pull-down, surface plasmon resonance of immobilised protein, the dissociation constant or complexin binding. This is discussed in more detail above.

Since the SNARE complex is well studied and is well known to those skilled in the art, a skilled person would be able to establish whether particular proteins are suitable for use in the present invention and how to manipulate these proteins and their sequences to produce the polypeptide helices of the invention so that they can form a stable SNARE complex.

The one or more cargo moiety may be any moiety which a skilled person might want to attach to the polypeptide helices. The one or more cargo moiety is preferably attached to the end of the helices in the complexing system. The cargo moiety may be selected from a small molecule, a polymer containing a small molecule, a polypeptide, a protein, a nucleic acid or derivative, and a particle or nanoparticle. For example, the cargo moiety may be:

1. a small molecule or a polymer containing a small molecule such as:
an affinity tag, e.g. biotin;
a therapeutic, e.g. a toxin or a drug;
a reactive group for further/downstream cross-linking, polymerisation and further derivatisation, e.g. an amino group, carboxyl group, sulfhydryl group, guanidine group, phenolic group, thioether group, imidazol group, indol group, etc.;
a spontaneously reactive group suitable for further modification, e.g. a maleimide or derivative for cross-linking to SH groups, or any other chemistry suitable for cross-linking;
a molecule for direct attachment to surfaces, e.g. an SH— containing molecule for attachment to metal surfaces;
an imaging reagent, e.g. a fluorescent or absorbent moiety for UV, VIS, IR, Raman, NMR, MRI, PET, X-ray or other imaging;
a biologically relevant ligand, e.g. for receptor binding/targeting;
a biologically relevant substrate, e.g. a phosphorylation or other PTM site;
a biologically relevant molecule, e.g. a lipid or carbohydrate;
a protective group or molecule, e.g. PEG;
a metal-chelating compound;

2. a polypeptide or protein such as:
a binding peptide, hormone, toxin, etc.;
a polypeptide or protein containing a functional site, e.g. a protease digestion site;
a targeting functional peptide, e.g. for different organelle targeting, nuclear targeting (for transfection), intracellular targeting (for drug delivery), etc.;
a peptide affinity tag, e.g. Flag, Myc, VSV, HA, 6×His, 8×his, poly-His, etc.;
a polypeptide or protein capable of forming a protein-protein interaction, e.g. PDZ, SH2/3;
an antibody, antibody fragment, antibody mimic, RNA- or peptide-based aptamer, or another affinity reagent (proteinous or non-proteinous);
an enzyme, e.g. for research, diagnostics (the complexing system can be used to immobilise enzymes for some applications) and therapeutic applications, for nucleic acid synthesis or amplification including promoters, polymerases, restriction endonucleases, or other modifying enzymes;

3. a nucleic acid or derivative such as:
DNA, RNA, or PNA for detection, immobilisation, hybridisation, synthesis priming, synthesis and amplification, labelling, signal detection and signal amplification, transcription and translation; and

4. a particle or nanoparticle such as:
a ferromagnetic particle or nanoparticle (for separation);
dendrimers (for labelling);
a metallic particle or nanoparticle, e.g. gold or silver for staining or labelling;
a semiconductor particle or nanoparticle, e.g. quantum dots for labelling and detection;
a polymer micro or nanoparticle, e.g. resins, gels, etc.
a carbon nanotube or nanowire

In one embodiment, a plurality of cargo moieties can be attached to the ends of the polypeptide helices. The number of cargo moieties that can be attached to the ends of polypeptide helices will depend on how many free ends are present on the helices. For example, where the complexing system comprises four separate polypeptide helices, the helices will have eight free ends (one at each end of each helix). Therefore, it is possible to attach a cargo moiety to each of the eight free ends. This means that 1, 2, 3, 4, 5, 6, 7 or 8 cargo moieties could be attached to the free ends of the helices. Where some of the helices do not have free ends, for example, if two of the helices are joined together or one helix is attached to a substrate, the number of free ends is reduced. This will reduce the number of cargo moieties that can be attached to the ends of the helices. In one embodiment, two or more cargo moieties can be attached at an end of a helix.

In a particular embodiment, a first cargo moiety is attached to the end of a first helix and a second cargo moiety is attached to the end of a second helix. In such an embodiment, the first and second cargo moieties should not be attached together on the same helix or helix containing component. They should be attached to separate helices or helix containing components. For example, the two cargo moieties may be attached to two single independent helices.

When a polypeptide helix derived from syntaxin is joined to a polypeptide helix derived from synaptobrevin or a homolog thereof (described in more detail later), the first and second cargo moieties should not both be attached to this bi-helical component. Instead, the first cargo moiety may be attached to the synaptobrevin/syntaxin fusion protein and the second cargo moiety may be attached to one of the helices derived from a SNAP protein.

When the four polypeptide helices of the complexing system are joined together to form two helix containing components (described in more detail later), the first cargo moiety should be attached to the first helix containing component and the second cargo moiety should be attached to the second helix containing component.

In one embodiment, the first cargo moiety is an enzymatic or imaging moiety. In another embodiment, the second cargo moiety is a ligand for targeting the complexing system to a particular target, for example, a particular type of cell or a particular receptor. Preferably, in the same embodiment, the first cargo moiety is an enzymatic or imaging moiety and the second cargo moiety is a ligand for targeting the complexing system to a particular target, for example, a particular type of cells or a particular receptor.

The enzymatic or imaging agent may be any suitable agent. The imaging agent can be any agent which can be attached to a helix and which allows the position of the helix to be imaged, for example, a GFP fluorescent tag, fluorescently labelled peptides, and MRI contrast agents. The enzymatic agent can be any enzyme or functional portion thereof. In one embodiment, the enzymatic or imaging agent is an enzymatic agent. In a specific embodiment, the enzymatic agent comprises the light chain of a botulinum toxin or a functional portion thereof. The function of the light chain of a botulinum toxin is as an endopeptidase. Therefore, a functional portion of the light chain of a botulinum toxin is a portion which retains the endopeptidase activity. Preferably, the enzymatic agent comprises the light chain of a botulinum toxin. The light chain of the botulinum toxin can be from any botulinum toxin. There are seven different types of botulinum toxin which are A, B, C, D, E, F and G. Preferably, the light chain is from botulinum toxin A or E and, more preferably, the light chain is from botulinum toxin A.

Preferably, the enzymatic agent comprises the light chain of a botulinum toxin or a functional portion thereof and the translocation portion of the heavy chain of a botulinum toxin. The translocation portion of the heavy chain of the botulinum toxin allows the light chain associated with it to be released from a vesicle into the cytosol of a cell. A skilled person would readily understand what is meant by the term ‘the translocation portion of the heavy chain of the botulinum toxin’, which may also be referred to as the translocation domain. The translocation portion of the heavy chain of the botulinum toxin can be from any botulinum toxin. Preferably, the translocation portion is from botulinum toxin A or E and, more preferably, from botulinum toxin A. The light chain or functional portion thereof and the translocation portion can be from the same or different botulinum toxins. Preferably, the light chain or functional portion thereof and the translocation portion are from the same botulinum toxin. The light chain or functional portion thereof and translocation portion may be joined in any suitable way. Preferably, they are joined via a disulphide bond as in a naturally occurring botulinum toxin. If the light chain of a botulinum toxin or a functional portion thereof and the translocation portion of the heavy chain of a botulinum toxin are joined by a peptide bond between the amino acid chains, preferably, there is a nicking site in the amino acid sequence between the light chain of a botulinum toxin or a functional portion thereof and the translocation portion of the heavy chain of a botulinum toxin which is recognised by a protease to cause cleavage of the amino acid sequence between the two parts. In one embodiment, the nicking site is a thrombin site which can be cleaved by thrombin. The light chain of a botulinum toxin or a functional portion thereof and the translocation portion of the heavy chain of a botulinum toxin may be attached to one of the polypeptide helices derived from a SNAP protein.

The sequence of the light chain of the botulinum toxin may comprise the sequence as shown in SEQ ID NO. 70.

The sequence of the translocation portion of the heavy chain of the botulinum toxin may comprise the sequence as shown in SEQ ID NO. 71.

When the cargo moiety is a ligand, it can be any suitable ligand for targeting the complexing system to a particular target, for example, a particular type of cell or a particular receptor. Such ligands are well known to those skilled in the art. For example, the ligand may be capable of binding to a cell surface receptor. Such ligands could include neuropeptides such as substance P, neuropeptide Y and VIP, growth factors such as NGF and BDNF, and hormones such as pituitary hormones (e.g. ACTH, TSH, PRL, GH, endorphins, FSH, LH, oxytocin, ADH and AVP), GNRH and CGRP. In a particular embodiment, the ligand is a somatostatin peptide or a functional portion thereof which allows the somatostatin peptide to bind to a somatostatin receptor. In other embodiments, the ligand may be a substance P peptide or a functional portion thereof which allows the substance P peptide to bind to a neurokinin receptor or the ligand may be an AVP peptide or a functional portion thereof which allows the AVP peptide to bind to an AVP receptor. In an alternative embodiment, the ligand may be the receptor binding portion of the heavy chain of a botulinum toxin. The receptor binding portion of the heavy chain of a botulinum toxin is responsible for recognition of neuronal gangliosides and binds to synaptic vesicle receptor, SV2C, allowing the toxin to be endocytosed into the cell. The term ‘receptor binding portion of the heavy chain of a botulinum toxin’ would be readily understood by a skilled person and may also be referred to as the receptor binding domain. The receptor binding portion can be from any botulinum toxin. Preferably, the receptor binding portion is from botulinum toxin A or E and, more preferably, the receptor binding portion is from botulinum toxin A. In one embodiment, the receptor binding portion of the heavy chain of a botulinum toxin is attached to the polypeptide helix derived from synaptobrevin or a homolog thereof.

The sequence of the receptor binding portion of the heavy chain of the botulinum toxin may comprise the sequence as shown in SEQ ID NO. 72.

The present invention also provides the above complexes for use in therapy and/or diagnosis. The exact nature of the therapy and/or diagnosis will depend on the identity of the ligand and enzymatic agent/imaging agent.

In one embodiment in which a first cargo moiety is attached to the end of a first helix and a second cargo moiety is attached to the end of a second helix, the first cargo moiety is an enzymatic agent comprising the light chain of a botulinum toxin or a functional portion thereof and the translocation portion of the heavy chain of a botulinum toxin, and the second cargo moiety is a ligand which is the receptor binding portion of the heavy chain of a botulinum toxin. In an alternative embodiment, the first cargo moiety is an enzymatic agent comprising the light chain of a botulinum toxin or a functional portion thereof and the translocation portion of the heavy chain of a botulinum toxin, and the second cargo moiety is a ligand which is a somatostatin peptide or a functional portion thereof.

The one or more cargo moiety may be joined directly to the end of the helix or may be attached via a linker. Suitable linkers are well known to those skilled in the art. For example, this may be done chemically or, if the cargo moiety is a protein or polypeptide, recombinantly. The cargo moiety may be attached via a linker. Such linkers are well known to those skilled in the art.

The four polypeptide helices may be four separate helices which are not joined together in any way until they form a SNARE complex. In one embodiment, two of the helices may be joined together so that the complexing system comprises three separate components (referred to hereinafter as a three component system). The two helices may be joined together in any suitable way as long as the two helices can assemble into the same stable SNARE complex. The two helices may be joined together by recombinant means or chemically coupled. The two helices may be joined together via a linker The term “join” means the linear linking of helices (this is in contrast to binding of helices which happens in parallel orientation akin to ‘zippering’). Joining two helices together helps to simplify the complexing system. Any two helices may be joined together. In one embodiment, the two polypeptide helices derived from a SNAP protein are joined together. The advantage of this is that a full length SNAP protein can be used which comprises two polypeptide SNARE helices in the protein, for example, a full length SNAP-25 protein. Alternatively, the polypeptide helix derived from syntaxin may be joined to the polypeptide helix derived from synaptobrevin or a homolog thereof.

In another embodiment, two of the helices are joined together whilst the other two helices are also joined together. This creates a complexing system comprising two separate components (referred to hereinafter as a two component system) and further simplifies the system. The two sets of helices may be joined together in any suitable way as long as they can still form a stable SNARE complex. For example, the two sets of helices may be joined together by recombinant means or chemically coupled. One or both sets of helices may be joined together via a linker. Any combination of helices can be joined together to form the two sets of two helices. Preferably, the two polypeptide helices which are derived from a SNAP protein are joined together and the helices derived from syntaxin and synaptobrevin or a homolog thereof are joined together. This allows the use of a full length SNAP protein such as SNAP-25.

In an alternative two component system, three of the helices can be joined together. The three helices may be joined together in any suitable way as long as they can still form a stable SNARE complex with the fourth helix. For example, the three helices may be joined together by recombinant means or chemically coupled. The three helices may be joined together with a linker between two of the helices or between all three helices. Any three helices can be joined together. Preferably, the two polypeptide helices which are derived from a SNAP protein are joined together along with a third helix, i.e. the helix derived from syntaxin or the helix derived from synaptobrevin or a homolog thereof. In a particular embodiment, the two polypeptide helices which are derived from a SNAP protein are joined together along with the polypeptide helix derived from synaptobrevin or a homolog thereof. In an alternative embodiment, the two polypeptide helices which are derived from a SNAP protein are joined together along with the polypeptide helix derived from syntaxin. In one embodiment, the cargo moiety is attached to the fourth single helix, for example, to syntaxin when the two polypeptide helices which are derived from a SNAP protein are joined together along with the polypeptide helix derived from synaptobrevin or a homolog thereof.

It will be apparent to those skilled in the art that these two component systems are binary reagents which can be used in affinity applications analogous to antibody-antigen interactions. For example, a FLAG epitope can be attached to a protein of interest and recognised by its cognate antibody much as a syntaxin-derived tag can be added to a protein of interest and recognised by a tri-helical construct of SNAP25 and synaptobrevin helices. The affinity of the interaction between the tri-helical SNARE construct and the lone SNARE tag may be such that is allows for immobilisation of the protein, or may be reversible for other applications. This is advantageous because the tri-helical construct is cheaper to produce than an antibody.

It is known that the head domain of syntaxin 3 protects its SNARE motif from SNARE assembly but certain detergents and/or lipids can ‘open’ syntaxin for SNARE assembly (Darios and Davletov (2006); Rickman and Davletov (2005)). Therefore, in one embodiment in which the syntaxin derived helix has a syntaxin head domain attached to it, the system further comprises a detergent, preferably a mild detergent, such as octylglucopyranoside to open the syntaxin molecule to allow the formation of a stable SNARE complex. This allows the syntaxin SNARE motif to be controlled and, therefore, allows regulation of the formation of a SNARE complex.

Furthermore, the system may further comprise a detergent regardless of whether the head domain of syntaxin 3 is attached to the polypeptide derived from syntaxin. The inventors have found that the presence of a detergent can help to promote the assembly of the SNARE complex in the present invention. Preferably, the detergent is a mild detergent. Some assembly takes place in the absence of a detergent but the presence of a detergent promotes more efficient assembly of the SNARE complex. Preferably, the detergent is at a concentration above the critical micellar concentration (CMC) which is the concentration at which the detergent starts to form micelles. Preferably, the detergent has a carbon chain with a length of 7-12 carbon atoms. Preferably, the detergent is not Triton X-100 or Thesit. A suitable detergent can be selected from the group consisting of MEGA 8, C-HEGA 10, C-HEGA 11, HEGA 9, heptyl glucopyranoside, octylglucopyranoside, nonylglucopyranoside, zwittergent 3-08, zwittergent 3-10, and zwittergent 3-12. Preferably, the detergent is octylglucopyranoside. The advantage of the system comprising a detergent, and in particular a mild detergent, is that it allows for controlled and faster assembly of the SNARE complex. The presence of a detergent could also help preferentially form the SNARE complex as other protein interactions are disrupted by the detergent.

In one embodiment, one of the helices can be immobilised on a substrate. Preferably, the helix is immobilised on a substrate. The helix can be immobilised in any suitable way and such ways are well known to those skilled in the art. The helix may be immobilised via a linker. The substrate can be any suitable substrate for immobilising the helix. For example, the substrate may be a surface, a matrix, a bead, a quantum dot, a resin, glass, a metal, a polymer, a microscope slide, an array or a nanotube such as a carbon naotube. In one embodiment, the helix that is immobilised on the substrate does not have a cargo moiety attached to it.

In another embodiment, the complexing system can further comprise one polypeptide helix derived from complexin. The complexin protein from which the polypeptide helix is derived can be any complexin protein which can bind to a SNARE complex. The skilled person is aware of the various complexin proteins which can bind to a SNARE complex. For example, the complexin protein may be selected from mammalian complexin 1 or complexin 2 isoforms. Other preferable features and properties (e.g. size) of the helix derived from the complexin protein are the same as for the other helices. The helix derived from complexin may optionally carry one or two cargo moieties. As the complexin moiety only binds to a formed SNARE complex, it can also be used to purify specifically an assembled complex.

The helix derived from complexin may comprise the sequence as shown in SEQ ID NO. 63. The helix derived from complexin may consist of this sequence.

An alternative embodiment of the invention is directed to a SNARE complex in which the polypeptide helix derived from syntaxin is joined to the polypeptide helix derived from synaptobrevin or a homolog thereof. In this embodiment, there is provided a complexing system for forming a molecular scaffold, the system comprising:

two polypeptide helices derived from a SNAP protein;

one polypeptide helix derived from syntaxin;

one polypeptide helix derived from synaptobrevin or a homolog thereof; and

one or more cargo moieties attached to the polypeptide helices,

wherein the four polypeptide helices can form a stable SNARE complex and wherein the polypeptide helix derived from syntaxin is joined to the polypeptide helix derived from synaptobrevin or a homolog thereof.

This embodiment of the invention is directed to the three component system described above in which the polypeptide helix derived from syntaxin is joined to the polypeptide helix derived from synaptobrevin or a homolog thereof. It will be apparent to one skilled in the art that virtually all the description above relating to limitations and preferable features of the complexing system containing a helix which is less than 50 amino acids in length is equally applicable to the above system containing a polypeptide helix derived from syntaxin joined to a polypeptide helix derived from synaptobrevin or a homolog thereof. For example, the two helices derived from a SNAP protein in this system may also be joined together as described above to produce a two component system. The skilled person would appreciate that two of the helices do not need to be less than 50 amino acids in length.

In another embodiment of the invention which is directed to the two two-component systems described above, there is provided a complexing system for forming a molecular scaffold, the system comprising:

two polypeptide helices derived from a SNAP protein;

one polypeptide helix derived from syntaxin;

one polypeptide helix derived from synaptobrevin or a homolog thereof; and

one or more cargo moieties attached to the polypeptide helices,

wherein the four polypeptide helices can form a stable SNARE complex and wherein the polypeptide helices are joined together to form two helix containing components.

As described above, the two component system can be formed in two ways. First, two of the helices can be joined together and the other two helices can be joined together to form two components each comprising two helices. Alternatively, three of the helices can be joined together to give a component containing three helices which can form a SNARE complex with the single remaining helix.

Virtually all the description above of the limitations of the complexing system containing a helix which is less than 50 amino acids in length is equally applicable to the above system containing two components. It is clear to a skilled person which parts are applicable to the two component system. For example, the two component system does not need to have two helices which are less than 50 amino acids in length.

In another embodiment, the invention provides a complexing system for forming a binary compound comprising two cargo moieties, the complexing system comprising:

two polypeptide helices derived from a SNAP protein;

one polypeptide helix derived from syntaxin; and

one polypeptide helix derived from synaptobrevin or a homolog thereof,

wherein the four polypeptide helices can form a stable SNARE complex, wherein a first cargo moiety is attached to a first helix and a second cargo moiety is attached to a second separate helix, and wherein formation of the SNARE complex causes formation of the binary compound.

The term “binary compound” means a compound which is made up of two separate parts (cargo moieties) so that when the two parts are brought together, the compound is able to carry out its function. The function of the compound will depend on the functions or identities of the two separate parts of the compound. Preferably, the first cargo moiety has a first function and the second cargo moiety has a second function. As discussed above, the first cargo moiety may have an enzymatic or imaging function and the second cargo moiety may have a targeting function. This enables the compound to be targeted to a particular location at which the enzymatic or imaging part is able to carry out its function.

In one embodiment, the binary compound may be a polypeptide which is formed from distinct units. The polypeptide may be a toxin which is formed from distinct units. Suitable toxins are botulinum toxin, diptheria toxin, tetanus toxin and ricin. It will be apparent to a skilled person which peptides and toxins are suitable for use in the system of the invention. For example, the botulinum toxin is made up of three distinct portions: a receptor binding portion; a translocation portion; and an enzymatic portion. Therefore, the enzymatic portion and the translocation portion can form one part (cargo moiety) and the receptor binding portion can form a second part (cargo moiety) so that when they are brought together by the complexing system, a functional botulinum toxin is formed. Similarly, tetanus, ricin and diptheria toxin can be separated into two parts so that when they are brought together, a functional toxin is formed.

In one embodiment, the first cargo moiety has a first function and the second cargo moiety has a second function, which when brought together form a fully functional peptide, e.g. a toxin.

The selection of peptides which are formed from distinct units and can be used in the way described above is well within the capability of a person skilled in the art. For example, US2009/0035822 describes the formation of functional proteins from separate parts.

Diptheria toxin, tetanus toxin and ricin can be used in therapy, for example, in the treatment of neoplastic disease. Therefore, complexing systems involving these polypeptides can be used in therapy and in the treatment of neoplastic disease. They can also be used in a method of treatment comprising administering an effective amount of a composition comprising the complexing system to a subject.

The present invention also provides a method of forming a SNARE complex to form a binary compound comprising two cargo moieties, the method comprising:

binding together two polypeptide helices derived from a SNAP protein, one polypeptide helix derived from syntaxin, and one polypeptide helix derived from synaptobrevin or a homolog thereof to form a stable SNARE complex, wherein a first cargo moiety is attached to a first helix and a second cargo moiety is attached to a second separate helix, and wherein formation of the SNARE complex causes formation of the binary compound.

In another particular embodiment, the invention provides a complexing system for forming a botulinum toxin comprising:

two polypeptide helices derived from a SNAP protein;

one polypeptide helix derived from syntaxin; and

one polypeptide helix derived from synaptobrevin or a homolog thereof,

wherein the four polypeptide helices can form a stable SNARE complex, wherein a first cargo moiety is attached to a first helix and a second cargo moiety is attached to a second separate helix, and wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin.

This embodiment of the invention is directed to a particular application of the SNARE complex assembly in which a botulinum toxin can be produced in separate parts, thereby avoiding any risk associated with the complete toxin during the manufacturing process. The two parts can then be combined, using the formation of the SNARE complex, to form a functional botulinum toxin.

The invention also provides the use of the complexing system described above. Further, the invention provides the complexing system described above for use in therapy and also for use in treating diseases or conditions which are alleviated by the inhibition of neural synapses.

The skilled person will be fully aware of the diseases or conditions which are alleviated by the inhibition of neural synapses since the use of botulinum toxin A has been in widespread use for medicinal and cosmetic therapies for a number of years (see, for example, Jankovic (2004) Botulinum in clinical practice. J Neurol Neurosurg Psychiatry 75 951-957). In particular, some of the diseases or conditions which are alleviated by the inhibition of neural synapses are selected from the group consisting of: excessive sweating, excessive salivation, dystonias, gastrointestinal disorders, urinary disorders, facial spasms, strabismus, cerebral palsy, stuttering, chronic tension headaches, hyperlacrymation, hyperhidrosis, spasms of the inferior constrictor of the pharynx, spastic bladder, pain, migraine, and cosmetic treatments such as reducing wrinkles, brow furrows, etc.

A method of treatment is also provided, the method comprising administering an effective amount of the complexing system described above to a subject.

The present invention also provides a method of forming a SNARE complex to form a botulinum toxin, the method comprising:

binding together two polypeptide helices derived from a SNAP protein, one polypeptide helix derived from syntaxin, and one polypeptide helix derived from synaptobrevin or a homolog thereof to form a stable SNARE complex, wherein a first cargo moiety is attached to a first helix and a second cargo moiety is attached to a second separate helix, wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin.

This method causes the two parts of the botulinum toxin to be brought together thus producing a fully functional botulinum toxin.

Further, the present invention provides a component for forming a botulinum toxin, the component comprising: a polypeptide helix derived from: a SNAP protein; syntaxin; or synaptobrevin or a homolog thereof, wherein the polypeptide helix is attached to a cargo moiety comprising the light chain of a botulinum toxin or a functional portion thereof and the translocation portion of the heavy chain of a botulinum toxin. Also provided by the present invention is a component for forming a botulinum toxin, the component comprising: a polypeptide helix derived from: a SNAP protein; syntaxin; or synaptobrevin or a homolog thereof, wherein the polypeptide helix is attached to a cargo moiety comprising the receptor binding portion of the heavy chain of a botulinum toxin.

Additionally, the invention provides the use of the above components as well as a kit comprising two polypeptide helices derived from a SNAP protein; one polypeptide helix derived from syntaxin; and one polypeptide helix derived from synaptobrevin or a homolog thereof, wherein the four polypeptide helices can form a stable SNARE complex, wherein a first cargo moiety is attached to a first helix and a second cargo moiety is attached to a second separate helix, and wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin.

It will be appreciated by one skilled in the art that the embodiments described above relating to a botulinum toxin can comprise further limitations and that the limitations described elsewhere with regard to other aspects and embodiments of the invention are equally applicable to these botulinum toxin embodiments.

In one particular embodiment relating to the detergent, the invention provides a complexing system for forming a molecular scaffold, the system comprising:

two polypeptide helices derived from a SNAP protein;

one polypeptide helix derived from syntaxin;

one polypeptide helix derived from synaptobrevin or a homolog thereof; and

one or more cargo moieties attached to the polypeptide helices,

wherein the four polypeptide helices can form a stable SNARE complex and wherein the SNARE complex is formed in the presence of a detergent.

The various features and limitations associated with the complexing system described above are equally applicable to this embodiment of the invention.

Previously, it has been shown that arachidonic acid allows SNARE complex formation in the presence of Munc18 (Rickman C and Davletov B (2005)). Munc18 is thought to be a negative regulator of SNARE complex formation. The inventors have surprising found that a detergent allows better SNARE complex formation in the absence of Munc18. Preferably, in the system relating to the presence of a detergent, the system does not contain a Munc18 protein. The system is a Munc18 free system.

The present invention also provides the use of a detergent for promoting the assembly of a SNARE complex in the absence of Munc18. Preferably, the detergent is a mild detergent. Preferably, the detergent is a synthetic detergent. In one embodiment, the detergent has a carbon chain length of 7-12 carbon atoms.

All the embodiments of the invention described above relate to a complexing system in which a single stable SNARE complex is produced with one or more cargo moieties attached to that SNARE complex. This is useful in a large number of applications, for example, diagnostics. The system also has application in tagging applications for affinity purification of labelled proteins, immobilisation of proteins or cells, or identification of labelled proteins (ELISA, Western blot). Immobilisation of cargoes on substrates is of use in diagnostics and microarray applications. It will be apparent to those skilled in the art that the stability of the SNARE complex will be of particular use for immobilisation of cargoes in microfluidics or continuous flow applications. Further applications are described below:

In Solution Applications:
recombinant affinity reagent assembly, including combinatorial assembly, poly-, homo- and hetero-oligomerisation;
targeted delivery of functional cargo, e.g. drug delivery (drug=small molecules, nucleic acids, proteins). e.g. for the assembly of targeting and internalisation signals with the cargo;
tagging and labelling of protein molecules, molecular complexes containing protein molecules, cells, tissues, organs and organisms containing protein molecules;
for the assembly of binary compounds, e.g. functional proteins, enzymes, factors, co-factors, (and any other functional proteins), FRET labels, binary inorganic compounds and small molecules, binary organic compounds;
self-assembling protein structures and more complex assemblies containing proteins (e.g. spores)—for the post-assembly immobilisation of proteins onto the surface of the self-assembled structures.

Solid Surface Applications:
arrays, surface immobilisation;
immuno assays (e.g. ELISA) well plates surface modifications and protein immobilisation;
BIAcore and other SPR (surface plasmon resonance) instruments, surface modifications and protein immobilisation;
QCM (quartz crystal microbalance) instruments surface modifications and protein immobilisation;
MALDI Mass Spectrometry plate surface modifications and protein immobilisation;
microfluidic instrument surface modifications and protein immobilisation;
capillary electrophoresis surface modifications and protein immobilisation;
chromatography columns and stationary medium (e.g. beads) surface modifications and protein immobilisation;
scanning probe microscopy surface and tip modifications and protein immobilisation;
micro and nano-calorimetry instrument sensor surface modifications and protein immobilisation;
micro and nano-particle surface modifications;
solid surfaces (e.g. gold-plated glass slides), thin films, wires (e.g. nanowires) and nanotubes surface modifications.

Other Applications:
nanobiotechnology, e.g. “gluing” surfaces together with the biodegradable protein based self-assembling “glue”;
protein based fibres and polymers;
tissue scaffolds and tissue engineering.

Further, the present invention provides the use of any of the embodiments of the complexing system described above and, in particular, in any of the applications described above. For example, the present invention provides the use of the complexing system in diagnostics, such as an array, an assay, a microfluidic device, an SPR instrument, a QCM instrument, a mass spectrometer, an electrophoresis instrument, a chromatography column, a scanning probe microscope, or a calorimetry instrument. For example, the complexing system may be used in arrays to secure antibodies to a substrate.

In one embodiment, the present invention provides an apparatus having a stable SNARE complex immobilised thereon, the SNARE complex comprising:

two polypeptide helices derived from a SNAP protein;

one polypeptide helix derived from syntaxin;

one polypeptide helix derived from synaptobrevin or a homolog thereof; and

one or more cargo moieties attached to the polypeptide helices,

wherein the apparatus is selected from an array, an assay, a microfluidic device, an SPR instrument, a QCM instrument, a mass spectrometer, an electrophoresis instrument, a chromatography column, a scanning probe microscope, and a calorimetry instrument.

The present invention also provides a method of forming a SNARE complex carrying one or more cargo moiety, the method comprising:

binding together two polypeptide helices derived from a SNAP protein, one polypeptide helix derived from syntaxin, and one polypeptide helix derived from synaptobrevin or a homolog thereof to form a stable SNARE complex, wherein one or more cargo moiety is attached to the polypeptide helices and wherein:

(i) the polypeptide helix derived from syntaxin is joined to the polypeptide helix derived from synaptobrevin or a homolog thereof;

(ii) the polypeptide helices are joined together to form two helix containing components; or

(iii) at least two of the polypeptide helices are less than 50 amino acids in length.

The above description relating to the system of the invention and the limitations are equally applicable to the above method.

The present invention also provides a component comprising a polypeptide helix derived from syntaxin joined to a polypeptide helix derived from synaptobrevin or a homolog thereof and wherein the two joined helices can form part of a stable SNARE complex. In one embodiment, the two helices are joined together so that they can assemble into the same stable SNARE complex. Alternatively, the two helices may be joined together so that they cannot assemble into the same stable SNARE complex. In this alternative embodiment, the two helices should be able to assemble into different stable SNARE complexes. The two helix component of the invention comprising a helix derived from syntaxin and a helix derived from synaptobrevin or a homolog thereof may comprise one of the following sequences shown in SEQ ID NO. 12, 13, 14 and 73.

Syntaxin 3 (1-260)/Synaptobrevin 2 (1-84) (syntaxin with the head domain and a linker)—SEQ ID NO. 12

Syntaxin 3 (195-253)/Synaptobrevin 2 (1-84) (syntaxin without the head domain but with a linker)—SEQ ID NO. 13

Syntaxin 3 (1-253)/Synaptobrevin 2 (29-84) (syntaxin with the head domain and no linker)—SEQ ID NO. 14

Syntaxin 3 (195-253)/Synaptobrevin 2 (29-84) (syntaxin without the head domain and no linker):—SEQ ID NO. 73

The two helix component of the invention comprising a helix derived from syntaxin and a helix derived from synaptobrevin or a homolog thereof may consist of one of the above sequences.

The two helix component may further comprise one or more cargo moieties attached to the polypeptide helices.

The present invention also provides a component comprising two polypeptide helices derived from a SNAP protein and either a polypeptide helix derived from synaptobrevin or a polypeptide helix derived from syntaxin, wherein the three helices are joined together to form a tri-helical component and wherein the three joined helices can form part of a stable SNARE complex. In one embodiment, the third helix is derived from synaptobrevin. In another embodiment, the three helices are joined together so that they can assemble into the same stable SNARE complex.

A tri-helical component of the invention comprising SNAP-25 helices and a helix derived from synaptobrevin or a homolog thereof may comprise SEQ ID NO. 15. Further, the tri-helical component may consist of this sequence.

Alternatively, the three helices may be joined together so that the two SNAP helices can assemble into the same stable SNARE complex but the third helix cannot assemble into the same stable SNARE complex as the two SNAP helices. In this alternative embodiment, the third helix should be able to assemble into a different stable SNARE complex compared to the two SNAP helices. The tri-helical component may further comprise one or more cargo moieties attached to the polypeptide helices.

Furthermore, the present invention provides the use of the above components, for example, to form a stable SNARE complex for forming a molecular scaffold.

The present invention further provides a kit comprising a component comprising a polypeptide helix derived from syntaxin joined to a polypeptide helix derived from synaptobrevin or a homolog thereof and wherein the two joined helices can form part of a stable SNARE complex.

The kit may further comprise two polypeptide helices derived from a SNAP protein which can form a stable SNARE complex with the syntaxin/synaptobrevin derived helices. The two SNAP helices may be joined together.

In an alternative embodiment, the present invention provides a kit comprising a component comprising two polypeptide helices derived from a SNAP protein and either a polypeptide helix derived from syntaxin or a polypeptide helix derived from synaptobrevin, wherein the three helices are joined together to form a tri-helical component and wherein the three joined helices can form part of a stable SNARE complex.

The kit may further comprise a single polypeptide helix derived from the fourth SNARE protein. So, if the tri-helical component comprises two SNAP derived helices and a synaptobrevin or homolog derived helix, the kit may further comprise a single polypeptide helix derived from syntaxin which can form a stable SNARE complex with the joined SNAP/synaptobrevin derived helices. Alternatively, if the tri-helical component comprises two SNAP derived helices and a syntaxin derived helix, the kit may further comprise a single polypeptide helix derived from synaptobrevin or a homolog thereof which can form a stable SNARE complex with the joined SNAP/syntaxin derived helices.

The kits of the invention may further comprise suitable reagents for use with the kit.

Further features of the helices and the SNARE complex of the kit are as described above.

The complexing system can also be used to form multimers of SNARE complexes. This is a plurality of SNARE complexes joined together. In one embodiment, two of the helices are joined together in such a way so that they cannot form a stable SNARE complex in the same complex. However, the two helices should be joined together in such a way so that they can each form a stable SNARE complex but in different SNARE complexes. This may be done by joining the helices directly together without any kind of linker so that the two helices cannot take up the correct conformation in the same SNARE complex. Instead, each helix in such a 2-helical joint construct can complex with the three other polypeptide helices to form two stable SNARE complexes. The two helices can be joined together in any suitable way, for example, by recombinant means or chemical coupling. Take, for example, a two component system in which the two helices derived from a SNAP protein are joined together and the two helices derived from syntaxin and synaptobrevin are joined in a restricted manner so that they cannot assemble in the same SNARE complex. Starting with a syntaxin-synaptobrevin fusion protein, the SNAP fusion protein will complex with the syntaxin helix. A synaptobrevin helix from another syntaxin-synaptobrevin fusion protein will then bind to the SNAP-syntaxin complex to form a four-helical SNARE complex. This complex will have a syntaxin helix and a synaptobrevin helix protruding from it, one from each of the syntaxin-synaptobrevin fusion proteins. These single helix protrusions can be the basis for the formation of further SNARE complexes. Therefore, a multimer of SNARE complexes is formed. In the above example, the SNAP helices do not have to be joined together. Further, any two helixes could be joined together for the system to work as long as they both cannot assemble in the same complex to form a stable SNARE complex.

The present invention also provides a multimer produced by the above system.

Alternatively, a multimer can be formed by controlling the reaction conditions of the system. Two of the helices of the system must be joined together. This can be done in any suitable way. The two helices do not need to be restricted in any way as in the above example although they can be, if desired. The system further comprises a single helix which is derived from the same SNARE protein as one of the two joined helices. For example, in a system comprising a syntaxin-synaptobrevin fusion protein, the system further comprises a single syntaxin helix. In order to produce a multimer, the single syntaxin helix is introduced into a solution. This single helix may be free in the solution or immobilised on a substrate, as discussed above. Two helices derived from a SNAP protein (which may or may not be joined) are added to the solution. These bind to the syntaxin helix to form a syntaxin-SNAP tri-helical complex. After this, the syntaxin-synaptobrevin fusion protein is added to the solution. The synaptobrevin helix from this fusion protein binds to the tri-helical complex to form a stable SNARE complex whilst the syntaxin helix will remain unbound and will protrude from the SNARE complex. By going through the same steps as above repeatedly, this syntaxin protrusion can be used to form further SNARE complexes, thus producing a multimer. Since the above system requires the helices to be added in a particular order, it may be necessary to ensure that there are no unwanted molecules after a particular step. This can be done, for example, by immobilizing the single syntaxin helix and performing washing after each binding step.

The present invention also provides a multimer produced by the above system.

Further, the present invention provides a multimer comprising a plurality of stable SNARE complexes joined together, wherein each SNARE complex comprises:

two polypeptide helices derived from a SNAP protein;

one polypeptide helix derived from syntaxin; and

one polypeptide helix derived from synaptobrevin or a homolog thereof,

wherein a helix from one SNARE complex is joined to a helix from another SNARE complex to join the SNARE complexes together, and wherein one or more cargo moiety is attached to the polypeptide helices.

The multimer of the present invention can be linear. If it is linear, each SNARE complex will be attached to two other SNARE complexes on either side to form a long linear chain similar to a string of beads. Therefore, two of the helices in a SNARE complex will be attached to a helix in two different SNARE complexes. Obviously, the SNARE complexes at the end of the chain will only be attached to one other SNARE complex by one polypeptide helix.

Alternatively, the multimer may be branched. This can be done, for example, by joining a synaptobrevin derived helix to two syntaxin derived helices in any order. The synaptobrevin derived helix from this triple joined construct will attach to a syntaxin/SNAP derived tri-helical intermediate to form a SNARE complex with the two syntaxin derived helices left unbound. When a SNAP derived helices are added, for example, SNAP-25, two joint syntaxin/SNAP-25 intermediates will form. Upon addition of a ‘normal’ synaptobrevin-syntaxin (2-helical) derived construct, two joint SNARE complexes will form with two syntaxin derived helices protruding. This will seed two branches for further growth. Therefore, joining three helices allows branching (in contrast to two joint helices which are used for linear polymerization). Therefore, if a multimer is branched, one SNARE complex within the multimer will be attached to three other SNARE complexes to form a branch point rather than being attached to two other SNARE complexes, as in a linear chain. Three of the helices in the SNARE complex at the branch point will be attached to a helix in three different SNARE complexes.

The multimer can have a cargo moiety attached to the end of each of the helices. The multimer may have a plurality of cargo moieties attached to the multimer at the end of the helices. In one embodiment, the multimer may have a cargo moiety on each SNARE complex. The multimer may have a plurality of cargo moieties attached to each SNARE complex. If the multimer has a plurality of cargo moieties, these may be the same or different. By providing SNARE domains carrying different cargoes after each wash step, the addition and ordered array of multiple cargoes can be controlled.

The present invention also provides a method of producing a multimer, the method comprising the following steps:

1) providing a first polypeptide helix derived from a first SNARE helix;

2) binding a second and a third polypeptide helix to the first polypeptide helix to form a tri-helical intermediate complex, wherein the second and third polypeptide helices are derived from a second and a third SNARE helix;

3) binding a fourth polypeptide helix to the tri-helical bundle to form a stable SNARE complex, wherein the fourth polypeptide helix is derived from a fourth SNARE helix, and wherein the fourth polypeptide helix is joined to a fifth polypeptide helix derived from a first SNARE helix; and

4) repeating steps 2) and 3) to form a multimer,

wherein one or more cargo moieties are attached to the polypeptide helices.

The description above relating to the complexing system, the multimer and the various limitations are also applicable to the above method.

Steps 2) and 3) can be repeated as many times a necessary to produce a multimer of the required length.

The above method refers to a first, second, third and fourth SNARE helix. As described above, the SNARE complex is formed from four helices. Two of these helices are provided by a SNAP protein, one is provided by syntaxin and one is provided by synaptobrevin or a homolog thereof. Therefore, in the above method, these four helices, in no particular order, are the first, second, third and fourth SNARE helices. This is because the identity of a particular helix is not important as long as the four helices are used in the SNARE complex. Optionally, the SNARE complex may also be bound to complexin, as described above.

In one embodiment, the first helix is a syntaxin derived helix; the second helix is a SNAP derived helix; the third helix is a SNAP derived helix; the fourth helix is a synaptobrevin derived helix; and the fifth helix is a syntaxin derived helix.

Due to the fact that the four helices can be used in the SNARE complex in any order, the fifth polypeptide helix does not need to be derived from the same SNARE helix as the first polypeptide helix. Similarly, when forming the second, third, fourth, fifth, sixth, etc. SNARE complex in the multimer as the steps of the method are repeated, the second, third and fourth polypeptide helices do not need to be derived from the same SNARE helix as the second, third and fourth polypeptide helices in the first SNARE complex. The only requirement is that all four SNARE helices are represented in each SNARE complex so that a stable SNARE complex forms.

Since the above method involves a multi-step process, this allows the possibility of introducing different polypeptide helices in the subsequent repeated steps compared to the first steps as long as they form a stable SNARE complex. For example, in the first cycle of steps, one of the polypeptide helices derived from a SNAP protein could be a full length SNAP-25 helix whereas in the second cycle of steps, this polypeptide helix could be a SNAP-25 helix which is 45 amino acids in length. The important aspect is that both polypeptide helices are derived from the same SNARE helix, e.g. a particular SNAP helix, so that all four SNARE helices are represented in the SNARE complex.

One or more cargo moiety is attached to the polypeptide helices so that the resulting multimer has a cargo moiety attached to it. This can be done by attaching a cargo moiety to the polypeptide helices before being used in the method. Preferably, the cargo moiety is attached at the end of the polypeptide helix. A plurality of cargo moieties can be introduced into the multimer. This can be done by attaching a cargo moiety to a number of polypeptide helices which are then incorporated into the multimer using the above method. The cargo moieties may be the same or different.

In order to make the method simpler, it is preferable that the identity of the first, second, third and fourth SNARE helices are maintained in the repeated steps. So, for example, if, in the first cycle of steps which produce the first SNARE complex, the first SNARE helix is syntaxin, the second and third SNARE helices are from a SNAP protein, and the fourth SNARE helix is synaptobrevin or a homolog thereof, in subsequent cycles of steps which produce further SNARE complexes, the first SNARE helix is always syntaxin, the second and third SNARE helices are always from a SNAP protein, and the fourth SNARE helix is always synaptobrevin or a homolog thereof.

Further, it is preferable that the identity of the second, third, fourth and fifth polypeptide helices are maintained in the repeated steps. So, for example, if, in the first cycle of steps which produce the first SNARE complex, the second and third SNARE helices are from a SNAP protein, the fourth SNARE helix is from a synaptobrevin SNARE helix, and the fifth polypeptide helix is from a syntaxin, in subsequent cycles of steps which produce further SNARE complexes, the fifth polypeptide helix (which forms the first helix in the next SNARE complex) is always a syntaxin, the second and third SNARE helices are always from SNAP-25, and the fourth SNARE helix is always a synaptobrevin SNARE helix.

In one embodiment, the second and third polypeptide helices are joined together so that they can assemble together in the same SNARE complex. Preferably, the second and third polypeptide helices are a full length SNAP protein, e.g. SNAP-25.

When the identity of the polypeptide helices are the same in all subsequent steps and the second and third polypeptide helices are joined together, this allows rapid multimer formation using only two building blocks.

In another embodiment, the first polypeptide helix is immobilised on a substrate. Suitable substrates are well known to those skilled in the art and are discussed above.

The method may further comprise the step of washing after each binding step to remove any unbound helices. This ensures that these unbound helices cannot interfere with the formation of the multimer in subsequent steps. Preferably, the multimer that is being formed by the method is immobilised before a washing step is used.

The method may be modified to introduce a branch into the multimer. In order to do this, a sixth polypeptide helix derived from a first SNARE helix is joined to one of the second, third, fourth or fifth polypeptide helices to provide a helix upon which another stable SNARE complex can be formed. In one embodiment, the sixth polypeptide helix is joined to the second or third polypeptide helices. In an alternative embodiment, the sixth polypeptide helix is joined to the fourth or fifth polypeptide helix. This allows two further SNARE complexes to originate from a particular SNARE complex, thereby introducing a branch in the multimer.

This can be done, for example, by joining a synaptobrevin derived helix to two syntaxin derived helices in any order, as discussed above. Alternatively, a syntaxin helix can be joined to SNAP-25 (being the second and third helix) to start the formation of a second SNARE complex from the originating SNARE complex, meaning that the originating SNARE complex will be attached to three other SNARE complexes overall to form a branch point.

Further, multiple branches can be introduced at a particular SNARE complex by introducing further helices in addition to the sixth helix which are joined to the other helices of the SNARE complex. For example, a synaptobrevin derived helix can be joined to three or more syntaxin derived helices in any order. Alternatively, two or more syntaxin helices can be joined to SNAP-25 (being the second and third helix) to start the formation of a third, fourth, etc. SNARE complex from the originating SNARE complex, meaning that the originating SNARE complex will be attached to four or more other SNARE complexes overall to form a multiple branch point.

As many branches as necessary can be introduced into the multimer at one particular point and over the length of the multimer.

The present invention also provides a multimer produced by the above method.

The multimer described above is useful in a large number of applications, for example, diagnostics and the applications described below:

In Solution Applications:
recombinant affinity reagent assembly, including combinatorial assembly, poly-, homo- and hetero-oligomerisation;
targeted delivery of functional cargo, e.g. drug delivery (drug=small molecules, nucleic acids, proteins). e.g. for the assembly of targeting and internalisation signals with the cargo;
tagging and labelling of protein molecules, molecular complexes containing protein molecules, cells, tissues, organs and organisms containing protein molecules;
for the assembly of multimeric compounds, e.g. functional proteins, enzymes, factors, co-factors, (and any other functional proteins), FRET labels, multimeric inorganic compounds and small molecules, multimeric organic compounds;
self-assembling protein structures and more complex assemblies containing proteins (e.g. spores)—for the post-assembly immobilisation of proteins onto the surface of the self-assembled structures.

Solid Surface Applications:
arrays, surface immobilisation;
immuno assays (e.g. ELISA) well plates surface modifications and protein immobilisation;
BIAcore and other SPR (surface plasmon resonance) instruments, surface modifications and protein immobilisation;
QCM (quartz crystal microbalance) instruments surface modifications and protein immobilisation;
MALDI Mass Spectrometry plate surface modifications and protein immobilisation;
microfluidic instrument surface modifications and protein immobilisation;
capillary electrophoresis surface modifications and protein immobilisation;
chromatography columns and stationary medium (e.g. beads) surface modifications and protein immobilisation;
scanning probe microscopy surface and tip modifications and protein immobilisation;
micro and nano-calorimetry instrument sensor surface modifications and protein immobilisation;
micro and nano-particle surface modifications;
solid surfaces (e.g. gold plated glass slides), thin films, wires (e.g. nanowires) and nanotubes surface modifications.

Other Applications:
nanobiotechnology, e.g. “gluing” surfaces together with the biodegradable protein based self-assembling “glue”;
protein based fibres and polymers;
tissue scaffolds and tissue engineering.

The invention will now be described in detail, by way of example only, with reference to the figures in which:

FIG. 1 is a schematic representation of the molecular machinery which drives vesicle fusion in neurotransmitter release. The core SNARE complex is formed by four α-helices contributed by synaptobrevin, syntaxin and SNAP-25. Synaptotagmin serves as a calcium sensor and regulates intimately SNARE zipping during vesicle fusion.

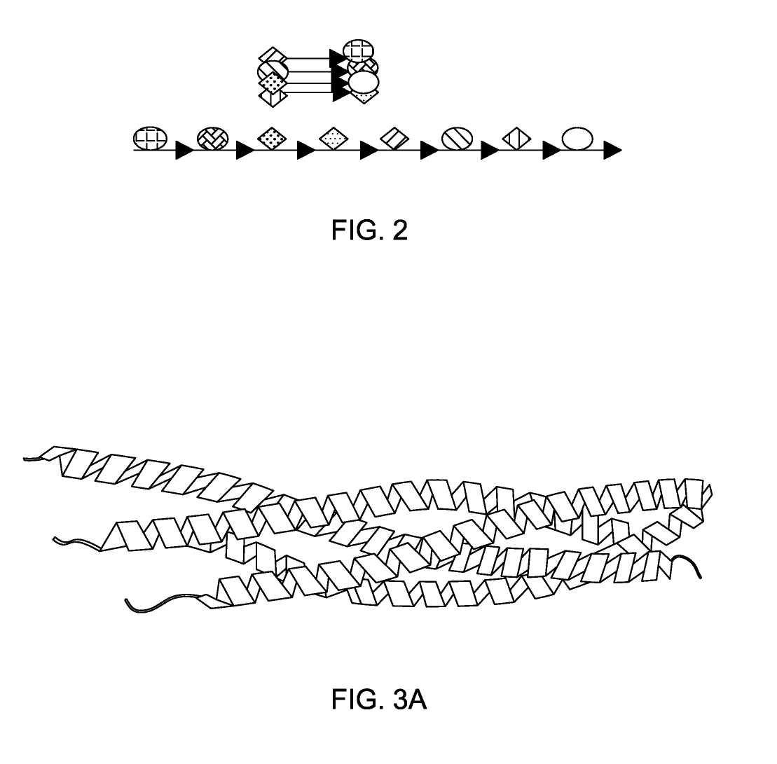

FIG. 2 is a schematic representation of linking of multiple functional units in an irreversible and site-specific manner as bundles or linear multimers. Arrows represent joining scaffold; functional units are represented by geometrical shapes.

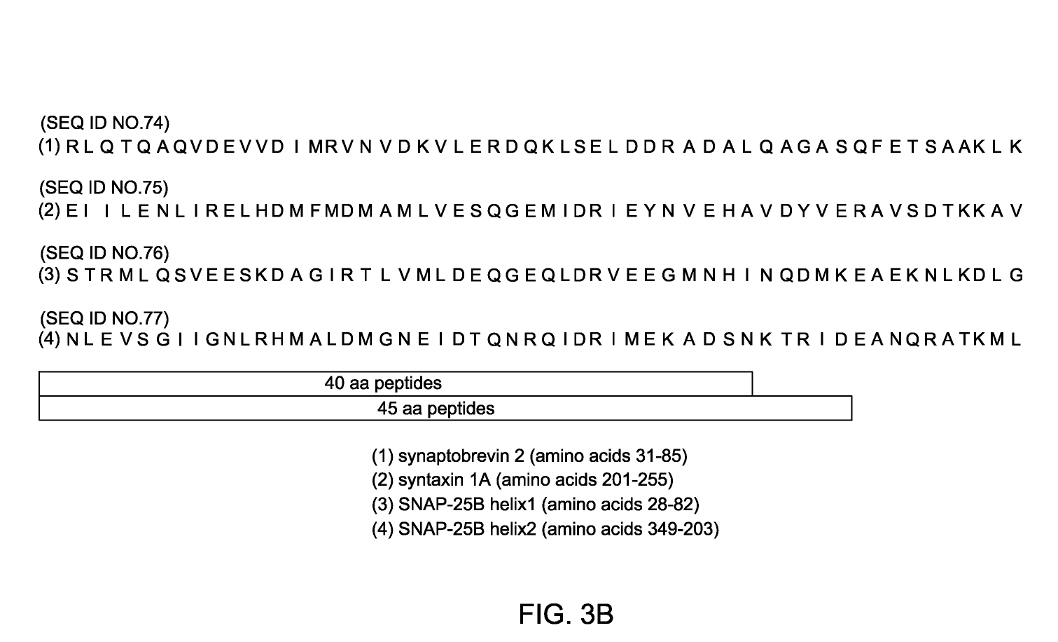

FIG. 3A shows the four-helical SNARE bundle made of four polypeptides with length of at least 80 amino acids (Sutton et al., 1998).

FIG. 3B shows SNARE motifs which were used for the design of 40 and 45 amino acid peptides. Shaded are hydrophobic layers, with the central layer highlighted in dark grey.

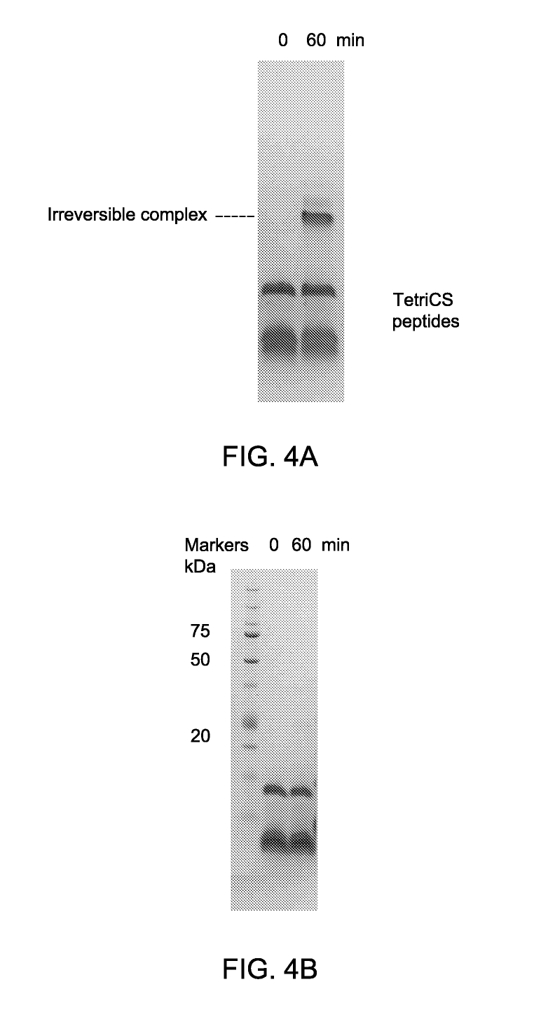

FIG. 4 is an SDS-PAGE gel showing that 45 amino acid polypeptides were able to form the irreversible SNARE complex (panel A), while 40 amino acids peptides did not (panel B). For brevity, the inventors call this assembly TetriCS (Tetrahelical Combinatorial Scaffold).

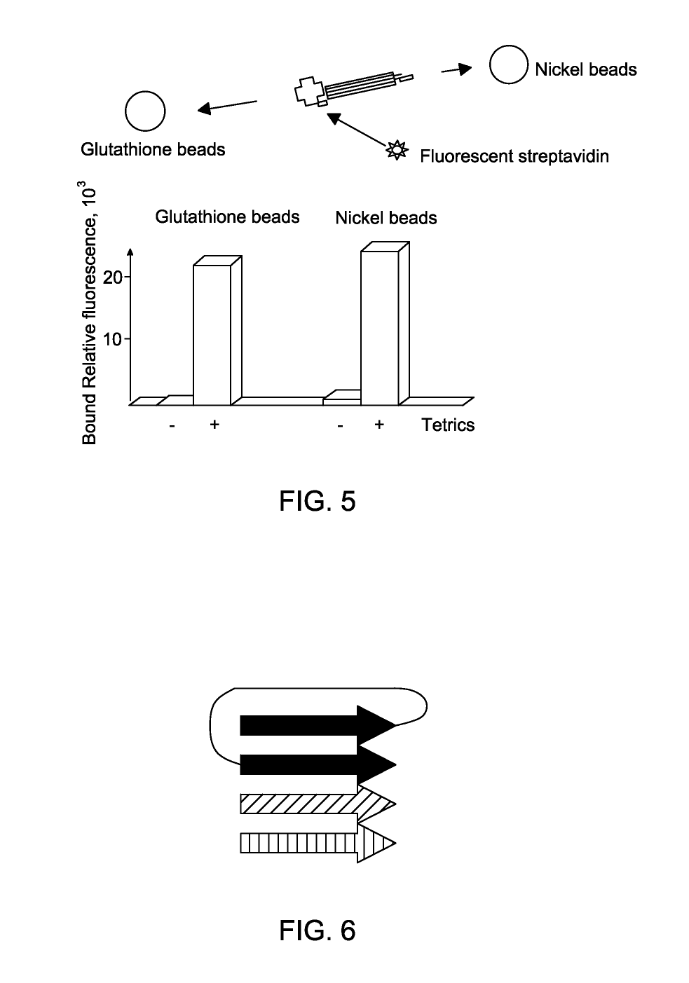

FIG. 5 is a graph showing that streptavidin could bind 45 aa Tetrics peptides attached to either glutathione or Nickel beads in a highly specific manner.

FIG. 6 is a schematic representation of a 3-component SNARE bundle comprising the full-length SNAP-25 molecule (amino acids 1-206) which has two SNARE helices and separate syntaxin and synaptobrevin SNARE helices.

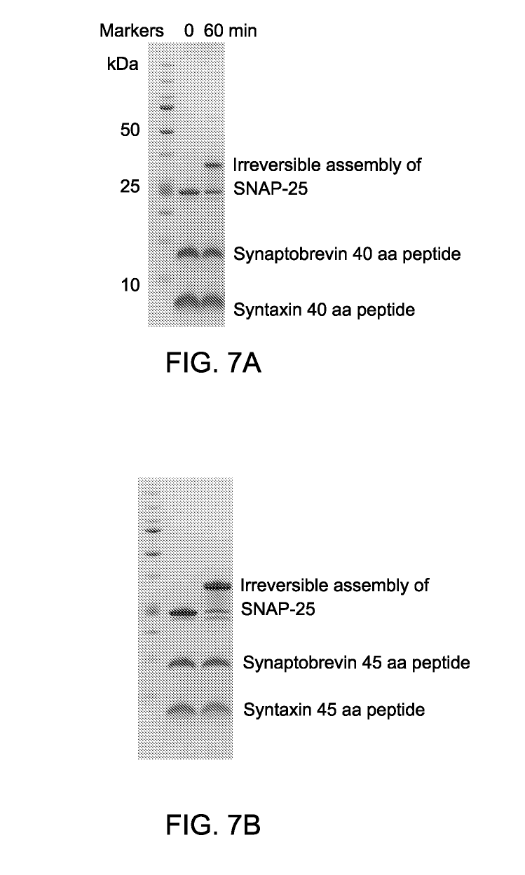

FIG. 7 is an SDS-PAGE gel showing that both 40 (panel A) and 45aa (panel B) peptides can assemble with SNAP25B, demonstrating that in the 3-component system, peptides of 40 aa length are sufficient for irreversible binding to full-length SNAP-25.

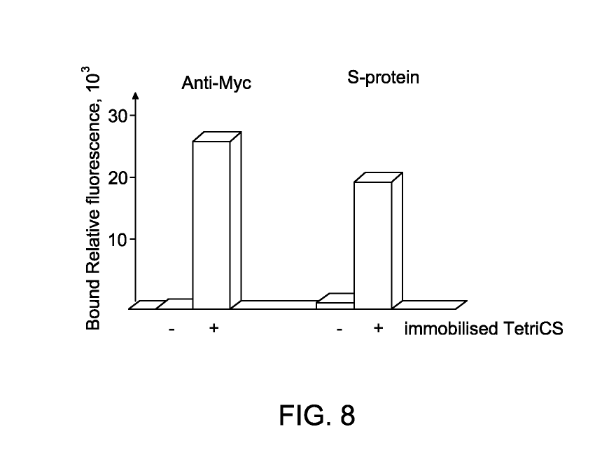

FIG. 8 is a graph showing that control reactions with GST-SNAP-25 alone on beads exhibited negligible binding, whereas addition of 40 aa syntaxin and synaptobrevin peptides, carrying Myc-tag and S-tag respectively, led to formation of 3-component complex as evidenced by a robust binding of both anti-Myc antibody and S-protein to glutathione beads.

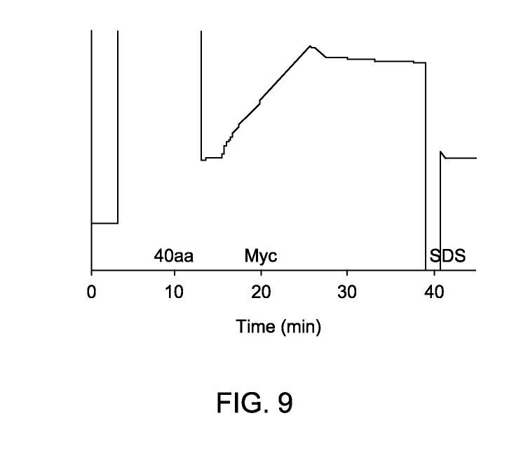

FIG. 9 is a sensogram showing binding of 40 aa syntaxin and synaptobrevin peptides to immobilized SNAP25B. Anti-myc antibody (Myc) binds to the syntaxin-myc epitope but can be eluted with 0.1% SDS. Bound syntaxin/synaptobrevin peptides cannot be dissociated by SDS.

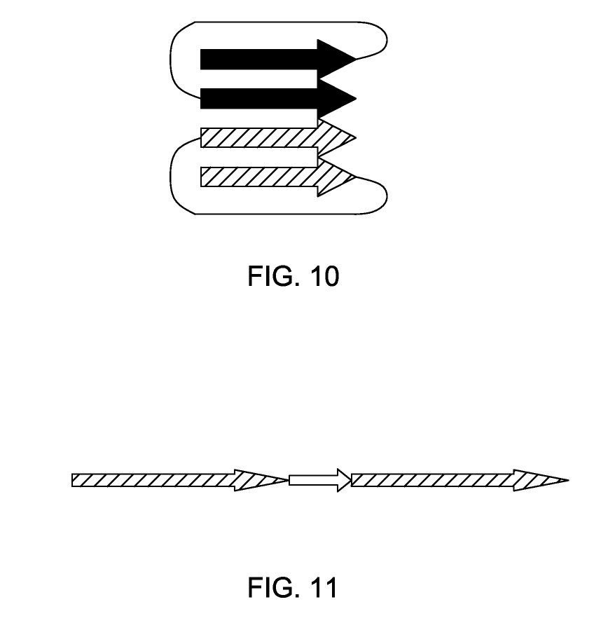

FIG. 10 is a schematic representation of a 2-component SNARE bundle comprising a SNAP-25 molecule, which has two SNARE helices, and a syntaxin/synaptobrevin fusion protein.

FIG. 11 is a schematic representation showing the linkage of the rat syntaxin1A SNARE motif (amino acids 195-254) with the rat synaptobrevin2 SNARE motif (aa 25-84) as illustrated by grey arrows.

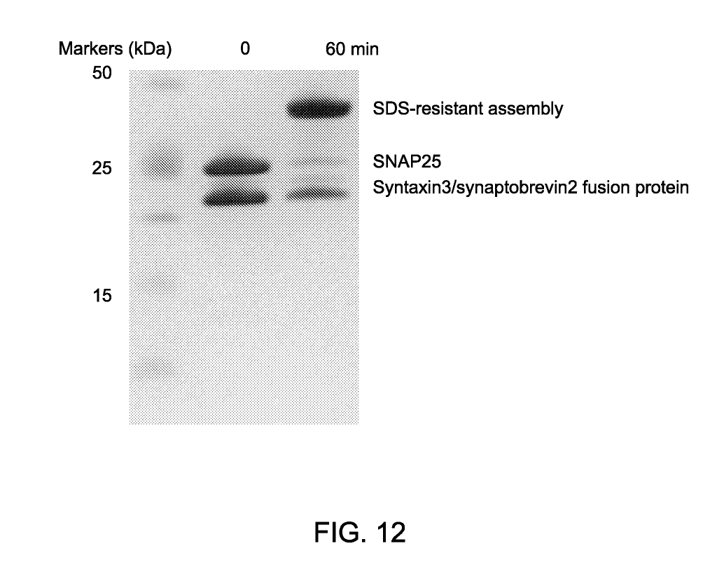

FIG. 12 is an SDS-PAGE gel showing that a syntaxin3-synaptobrevin2 fusion protein and rat SNAP-25B quickly assemble into an irreversible complex.

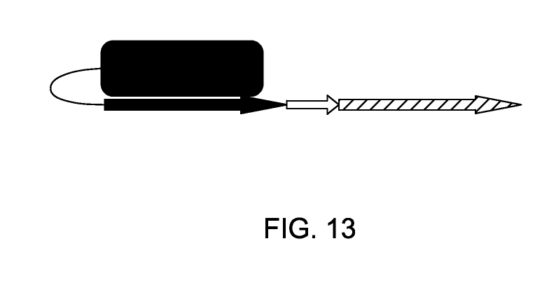

FIG. 13 is a schematic representation showing the head domain and the SNARE motif of rat syntaxin3 (amino acids 1-260) fused via a short stretch of amino acids to rat synaptobrevin2 sequence 1-84.

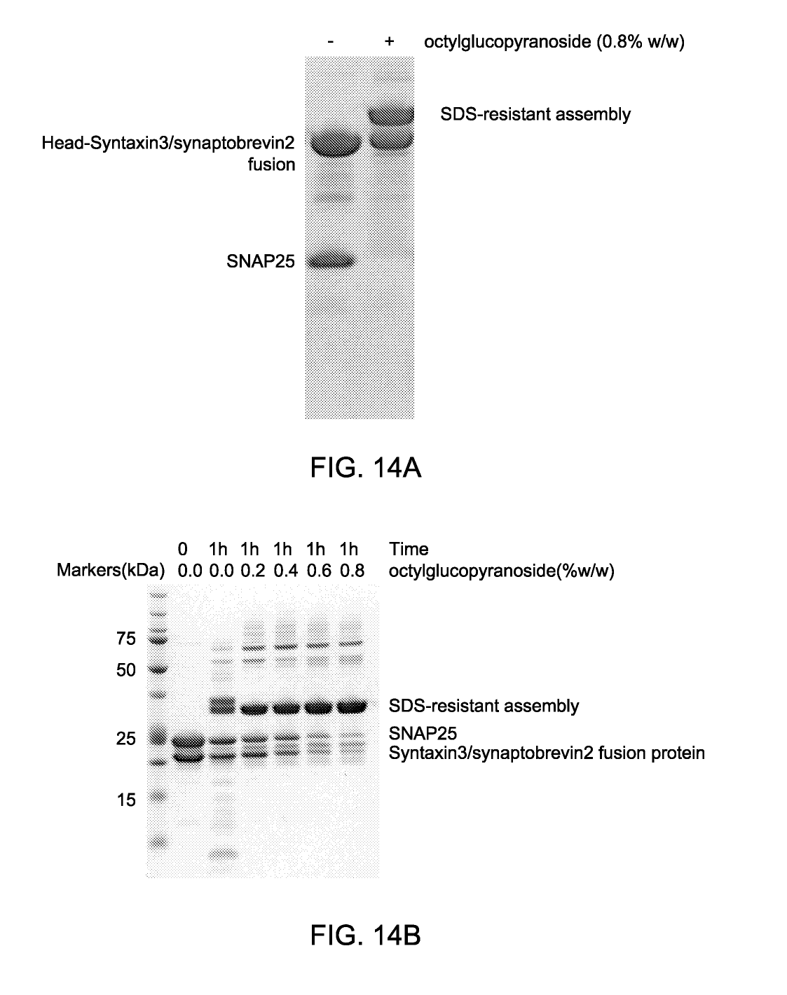

FIG. 14A is an SDS-PAGE gel showing that octylglucopyranoside can ‘open’ the syntaxin 3 molecule to allow a 2-component assembly to form.

FIG. 14B is an SDS-PAGE gel showing that octylglucopyranoside detergent promotes the assembly of a tight SNARE complex at concentrations above CMC (critical micellar concentration—for this detergent, the CMC is 0.6%). Some assembly does take place in the absence of the detergent, but efficient assembly requires the detergent octylglucopyranoside to ‘open’ the syntaxin 3 molecule to allow a 2-component assembly to form.

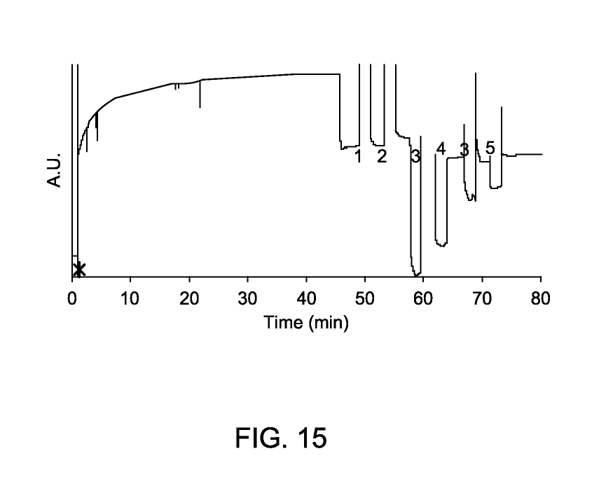

FIG. 15 is a Surface Plasmon Resonance Sensogram showing binding of syntaxin- synaptobrevin fusion protein (*) and the unique resistance of the assembly between the syntaxin/synaptobrevin fusion protein and SNAP-25 immobilized on the CM5 chip. Washes using 2M NaCl (1), glycine pH 2.5 (2), 1% SDS (3), 0.1 M NaOH (4), 0.1 M phosphoric acid (5) cannot break the binary capture reagent. Note, step 3 was repeated twice.

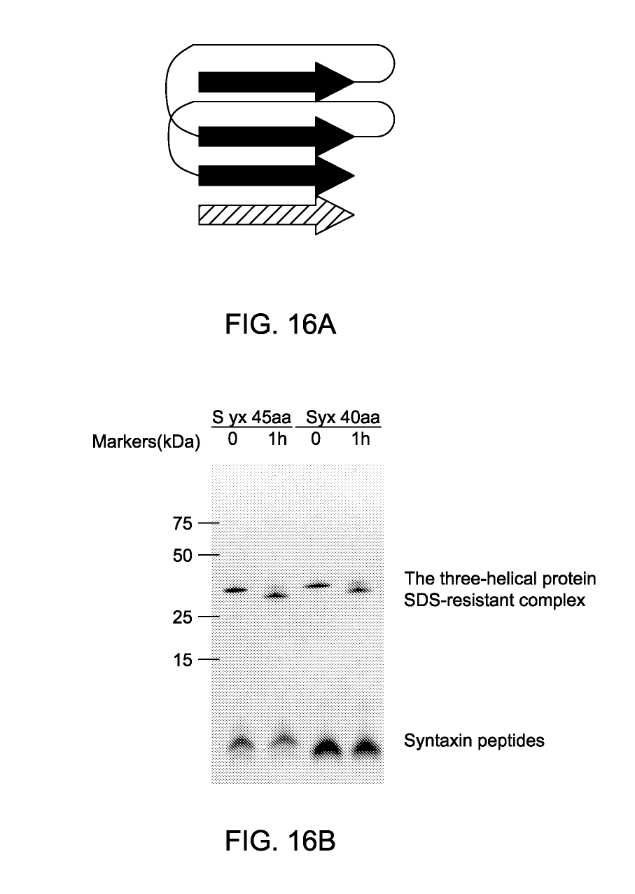

FIG. 16A is a schematic representation of a 2-component SNARE bundle comprising a three-helical molecule (black) and a forth helix (grey).

FIG. 16B is an SDS-PAGE gel showing that both 40 and 45aa syntaxin 1A peptides can assemble with the three-helical fusion protein composed of SNAP-25B and synaptobrevin 2 (SNAP-25B(22-206)/Syb2(1-84)) demonstrating that in a two-component system, peptides of 40 aa length are sufficient for irreversible SNARE complex formation. Note, the SDS-resistant complex migrates faster than the tri-helical protein in the SDS-gel, possibly due to its compact structure.

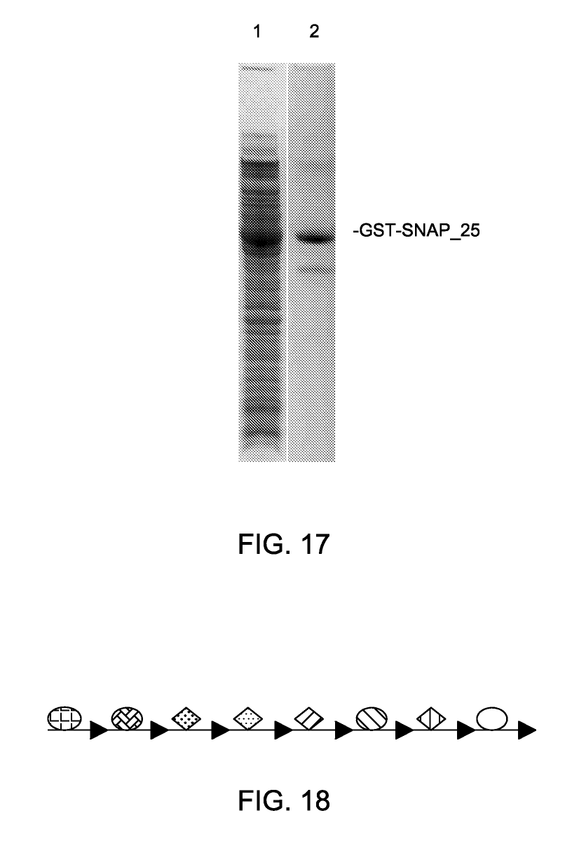

FIG. 17 is an SDS-PAGE gel showing that GST-SNAP-25 can bind to the Sepharose beads carrying syntaxin3/synaptobrevin2 fusion protein in a highly specific manner. Note, the syntaxin3/synaptobrevin2 fusion protein is covalently attached to BrCN-Sepharose beads and therefore cannot be eluted and visualised on the SDS PAGE gel. lane 1—bacterial extract produced using zwitergent 3-08. lane 2—GST-SNAP-25 purified on beads carrying syntaxin3/synaptobrevin2 fusion protein.

FIG. 18 is a schematic representation of a supramolecular device in the form of strong linear multimers of unlimited length formed by SNARE proteins.

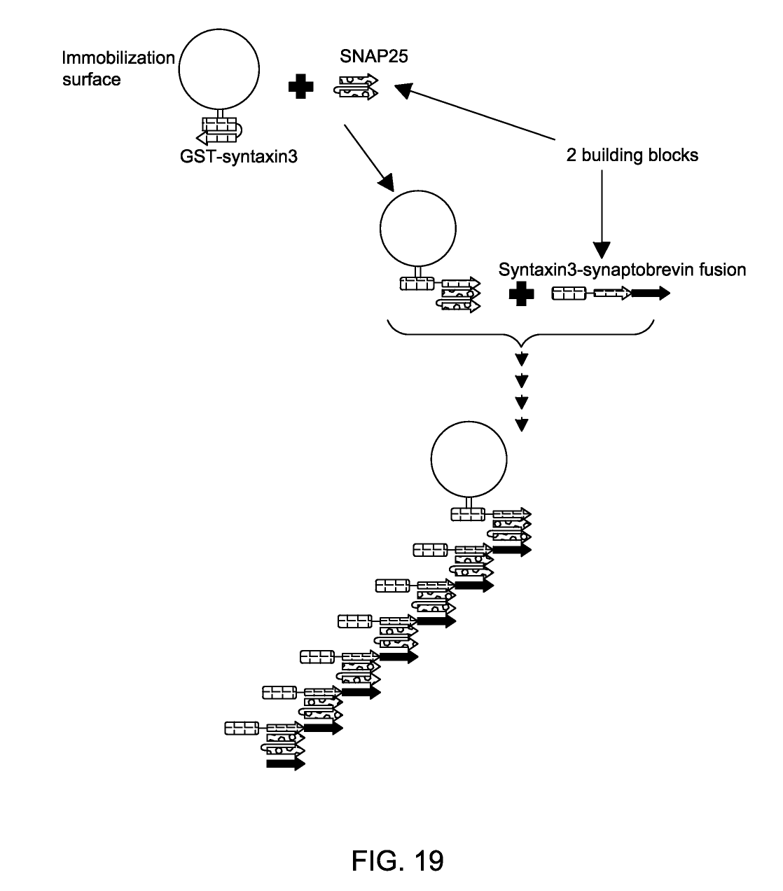

FIG. 19 is a schematic representation of the process of forming a long linear multimer using SNARE proteins.

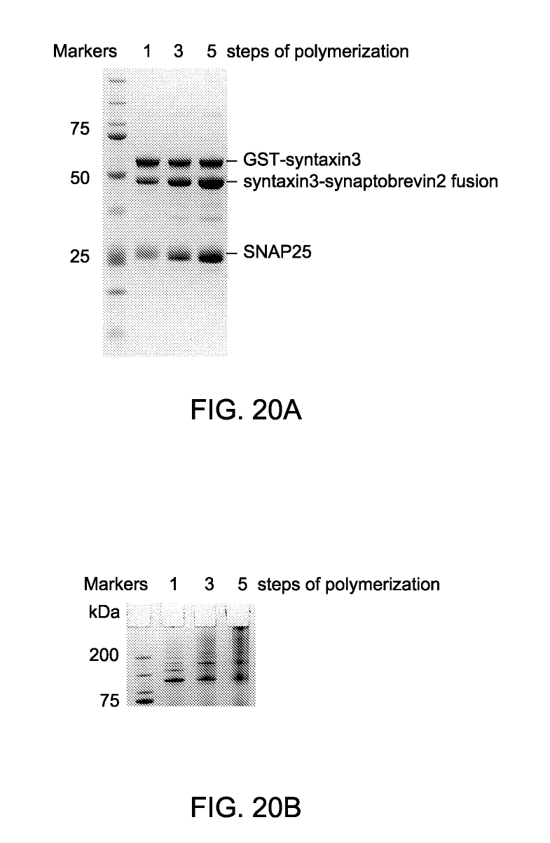

FIG. 20. (A) Coomassie-stained gel showing a step-wise increase in the amounts of both syntaxin3-synaptobrevin2 fusion and SNAP-25 bound to beads. Note, while the amount of GST-syntaxin3, used for attachment to beads, remains constant there is a gradual increase in the amounts of bound SNAP-25 and syntaxin3-synaptobrevin2 fusion protein. To show the amounts of bound material, the samples were boiled to disrupt SDS-resistant nature of the assemblies. (B) As in panel A, but samples were not boiled prior to SDS-PAGE. Note the increase in the molecular weight of SDS-resistant polymers in line with extra polymerization steps.

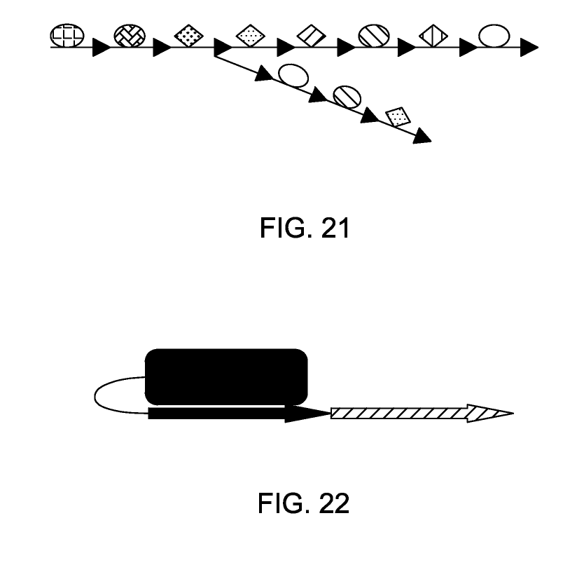

FIG. 21 is a schematic representation of a branched multimer formed from SNARE proteins.

FIG. 22 is a schematic representation of a fusion construct formed from syntaxin3 residues (1-253) directly fused to synaptobrevin2 residues (29-84) (no linker).

FIG. 23 is a SDS-PAGE gel showing that SNARE bundles can be multimerised in solution by simple mixing.

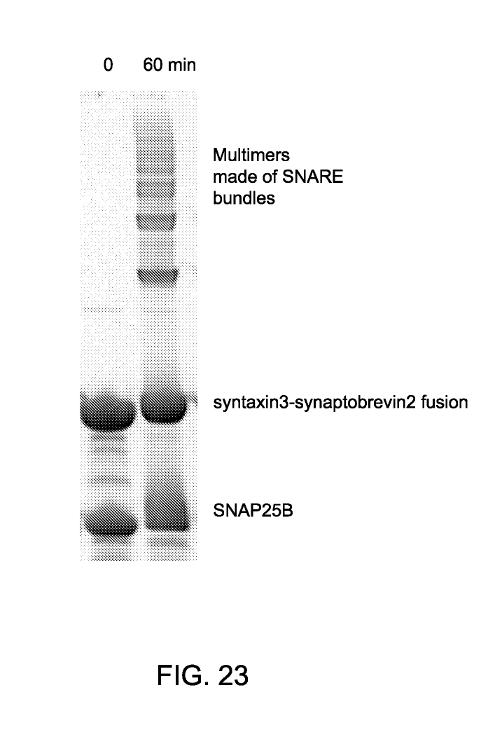

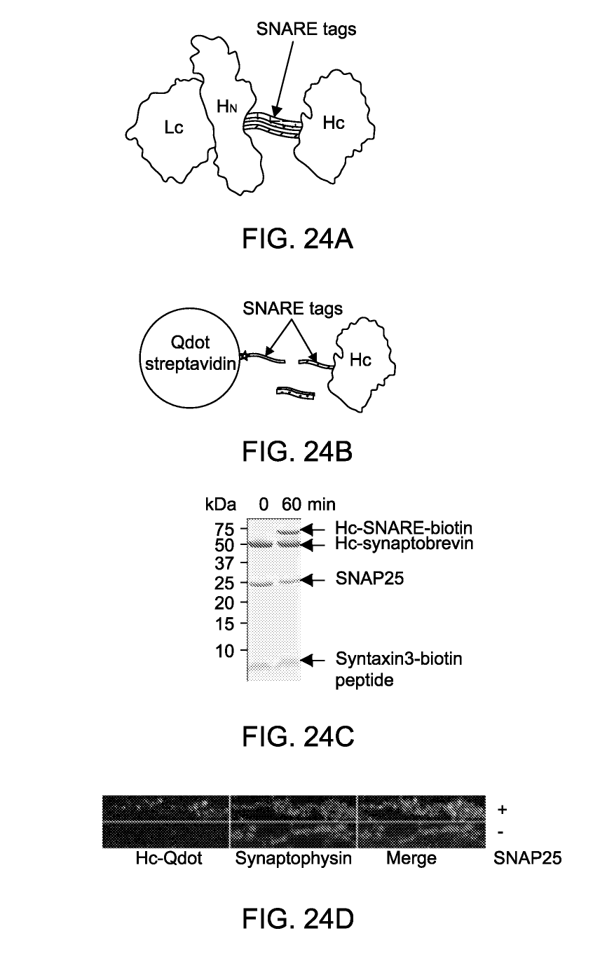

FIG. 24 shows that SNARE tagging allows Hc-mediated delivery of quantum dots to synaptic endings. a, Schematic showing SNARE linking of the LcHN part with the Hc part of BoNT/A. The individual subunits are shown as in the structural model (adapted from Lacy et al, 1998). b, Schematic showing the SNARE tagging scheme for linking streptavidin-coated quantum dot with the SV2C-binding part of botulinum neurotoxin (Hc). Biotin(star)-syntaxin peptide (blue) allows SNARE-tagging of the quantum dot, whereas Hc is fused to synaptobrevin SNARE motif (purple). SNAP25 (green and red) allows linking of Q-dot to Hc. c, Coomassie-stained SDS-gel showing an irreversible assembly of Hc-synaptobrevin, SNAP25 and biotinylated syntaxin3 peptide into an SDS-resistant complex, Hc-SNARE-biotin. d, Hc-SNARE-Q-dots exhibit synaptic binding as evidenced by the immunostaining for the synaptic vesicle marker synaptophysin at axonal extensions of cultured hippocampal neurons. Omission of SNAP25 during assembly prevents targeting of Q-dots to synaptic terminals.

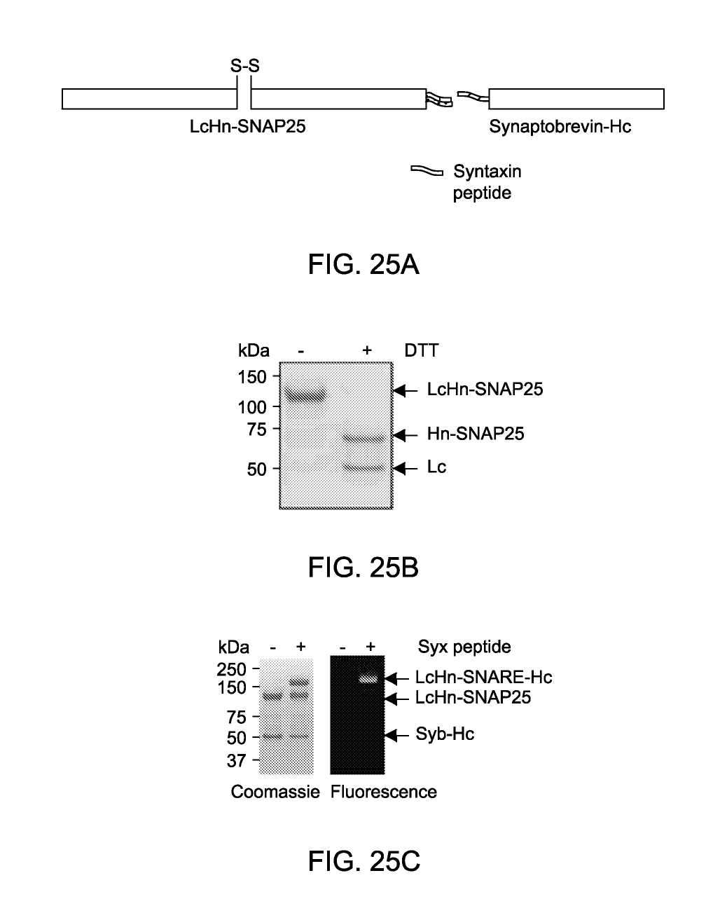

FIG. 25 shows that SNARE tagging allows a step-wise assembly of individual parts of BoNT/A into a single molecular entity. a, Diagram showing the position of the disulphide bond and SNARE tagging of LcHN and the He part of BoNT/A. b, LcHN, tagged with SNAP25, can be purified and broken into Lc and HN-SNAP25 following treatment with 50 mM dithiotreitol (DTT). Coomassie-stained SDS-gel. c, LcHN, tagged with SNAP25, can be united with Hc, tagged with synaptobrevin, upon addition of the syntaxin3 peptide as evidenced by the Coomassie-stained and fluorescently-imaged SDS-gels.

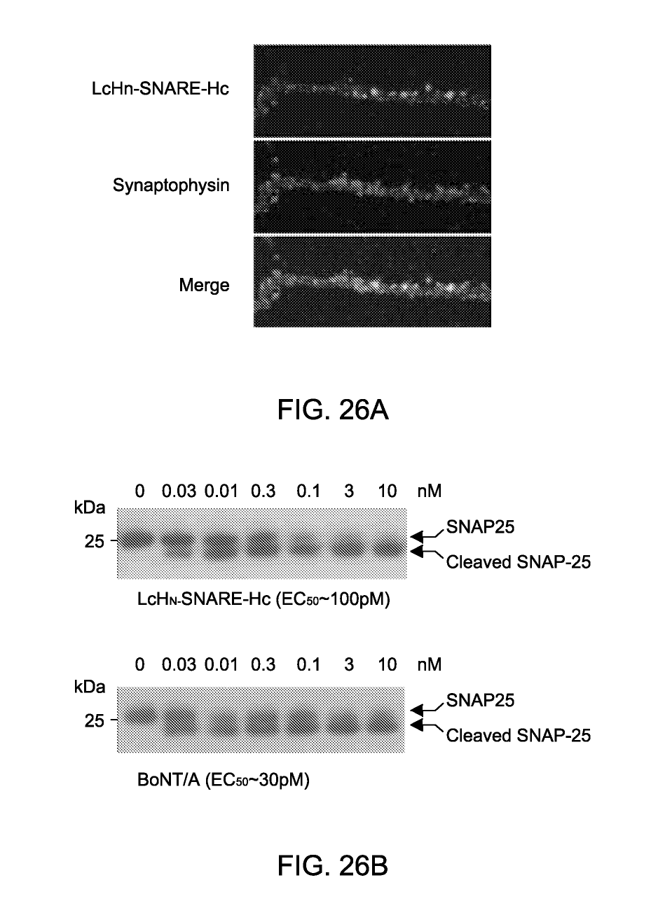

FIG. 26 shows that SNARE-linked botulinum neurotoxin exhibits synaptic localization and cleaves its intrasynaptic target. a, Fluorescein-labelled LcHN-SNARE-Hc binds to axonal extensions of hippocampal neurons. Immunostaining with anti-synaptophysin antibody highlights presynaptic terminals of cultured hippocampal neurons. b, Immunoblot showing cleavage of intrasynaptic SNAP25 by the assembled neurotoxin in a similar fashion as the native BoNT/A.

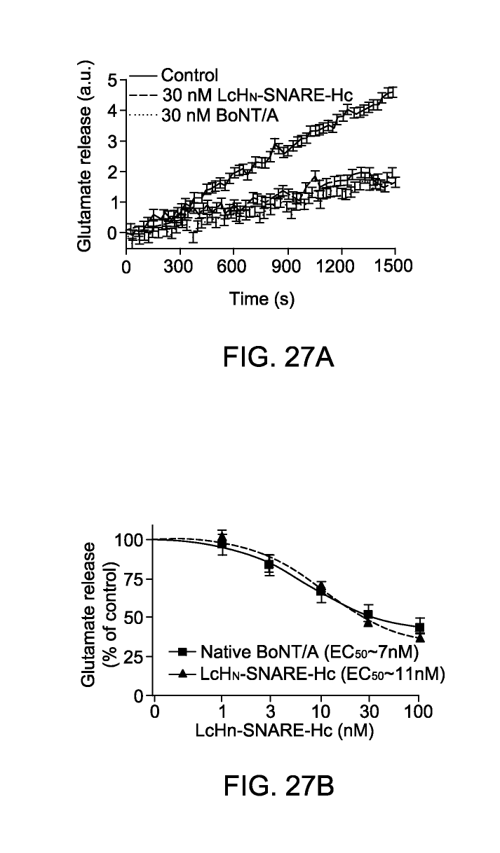

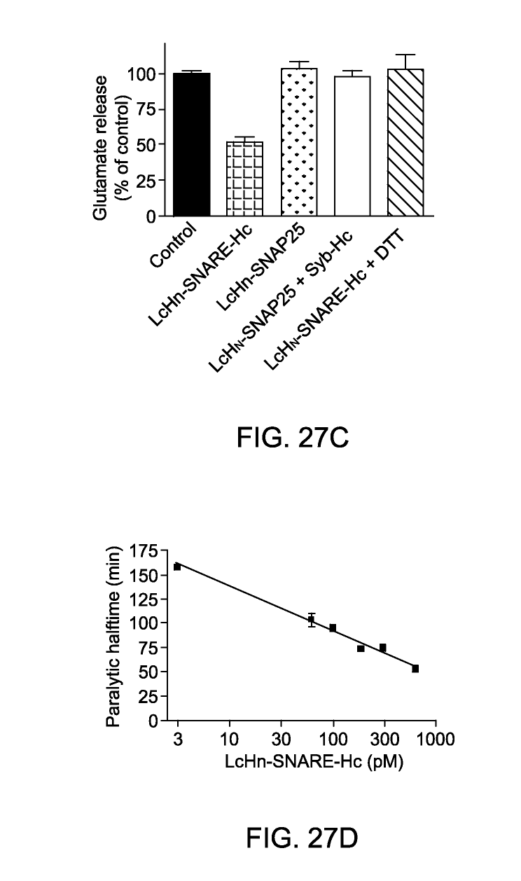

FIG. 27 shows that SNARE-linked botulinum neurotoxin inhibits neurotransmitter release. a, Fluorometric measurements of glutamate release from isolated rat brain synaptic endings (synaptosomes) indicate a similar degree of inhibition between LcHN-SNARE-Hc and BoNT/A. Real-time glutamate release graph (upper panel) and dose-dependence graph (bottom panel, assessed after 15 min stimulation with 35 mM KCl and 2 mM CaCl₂) were obtained following 1-hour incubation of synaptosomes with toxins. b, Individual SNARE-tagged neurotoxin parts do not block glutamate release, following 1 hour incubation with synaptosomes, as assessed after 15 min stimulation. c, Graph showing dose-dependent inhibition of isometric contractions of mouse diaphragm by the LcHN-SNARE-Hc. Error bars represent SEM; n=3.

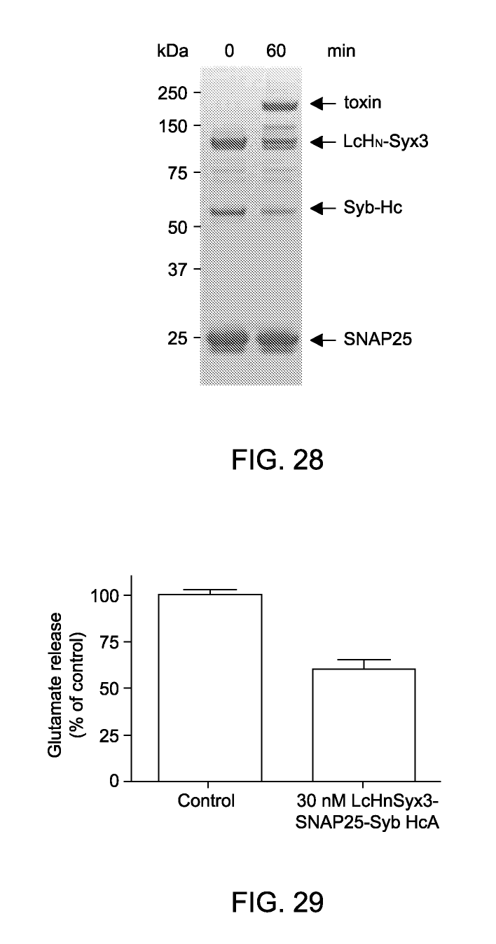

FIG. 28 shows LcHn was tagged by syntaxin3 (195-253) whereas He was tagged by synaptobrevin (25-84). The two botulinum parts were mixed in the presence of SNAP-25 and the toxin formation was visualised on Coomassie-stained SDS-gel.

FIG. 29 shows blockade of glutamate release from rat brain synaptosomes was assessed after 1 hour incubation with LcHnSyx3-SNAP25-synaptobrevinHc toxin from panel A.

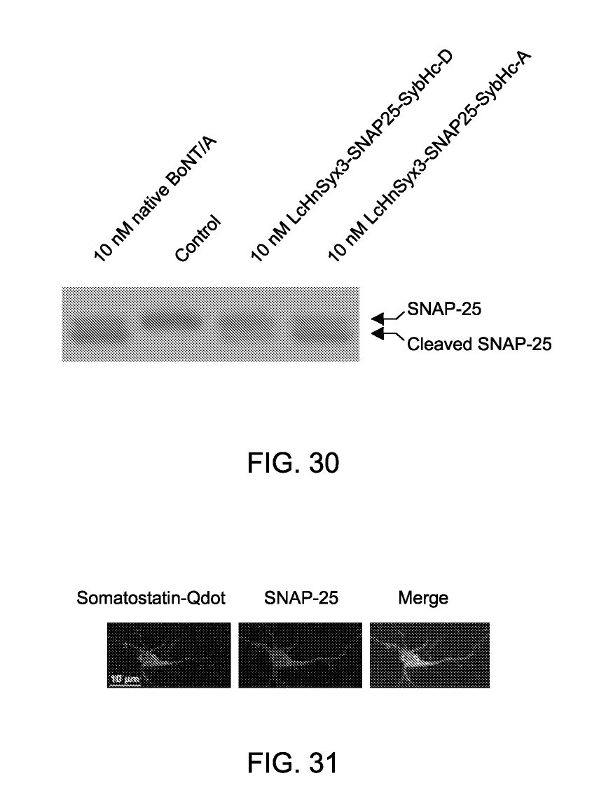

FIG. 30 shows cleavage of intraneuronal target of the catalytic part Lc after application of toxin LcHnSyx3-SNAP25-synaptobrevinHcA from panel A on hippocampal neurons was assessed by immunoblotting using anti-SNAP-25 antibody. The reassembled toxin has similar activity in cleaving SNAP-25 as the native botulinum neurotoxin (BoNT/A). Note, LcHnSyx3-SNAP25-synaptobrevinHcA is more efficient than LcHnSyx3-SNAP25-synaptobrevinHcD in this neuronal assay.

FIG. 31 shows SNARE tagging of synaptobrevin 40 amino acid motif with somatostatin peptide Ac-RLQQTQAQVDEVVDIMRVNVDKVLERDQKLSELDDRADAL-Ahx-Ahx-AGCKNFFWKTFTSC-OH allows making of somatostatin-quantum dots. Streptavidin-coated quantum dots were incubated with biotynylated syntaxin peptide and then mixed with somatotostatin-synaptobrevin and SNAP-25. The assembled somatostatin-Q-dots were applied on cultured hippocampal neurons and their entry into neuronal somas was visualised by fluorescence of Q-dots and counterstaining with anti-SNAP-25 antibody which labels the abundant intraneuronal protein.

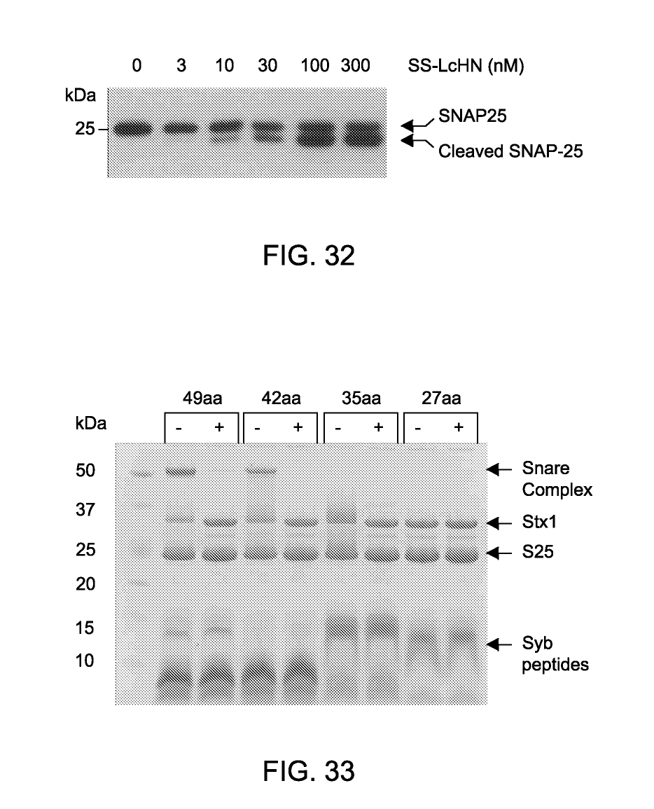

FIG. 32 shows SNARE tagging of synaptobrevin 40 amino acid motif with somatostatin peptide allows making of functional somatostatin-botulinum construct after mixing with LcHnsyntaxin3 and SNAP-25. The activity of the LcHnsyntaxin3-SNAP25-synaptobrevinSomatostatin (SS-LcHN) was assessed by cleavage of SNAP-25 in cultured hippocampal neurons after 20 hour application of the assembled toxin.

FIG. 33 is an SDS-gel showing formation of stable SDS-resistant complexes using SNARE motifs with N-terminal truncation.

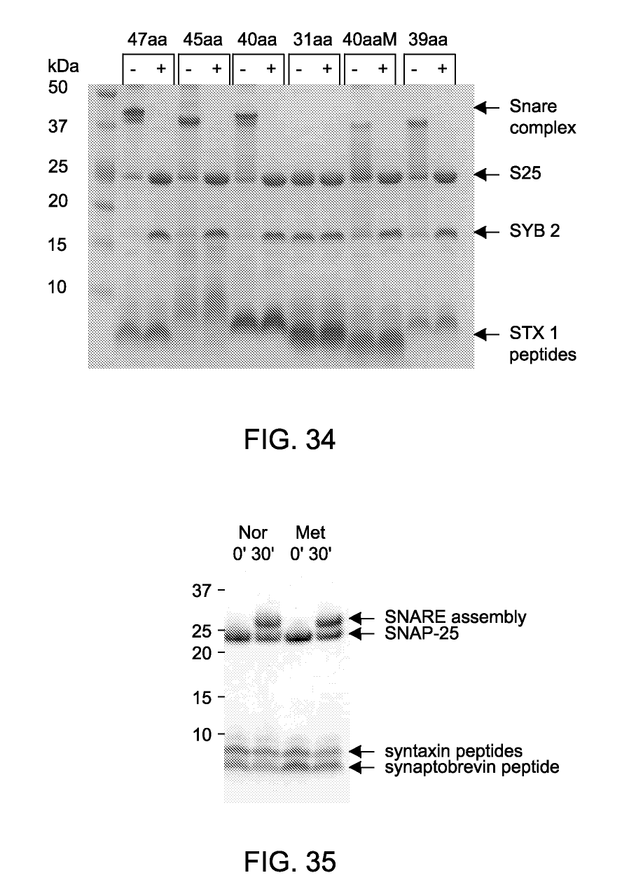

FIG. 34 is an SDS-gel showing formation of stable SDS-resistant complexes using SNARE motifs with C-terminal truncation.

FIG. 35 is an SDS-gel showing formation of stable SDS-resistant complexes using a syntaxin SNARE peptide in which the internal methionines have been replaced with non-oxidizable norleucines.

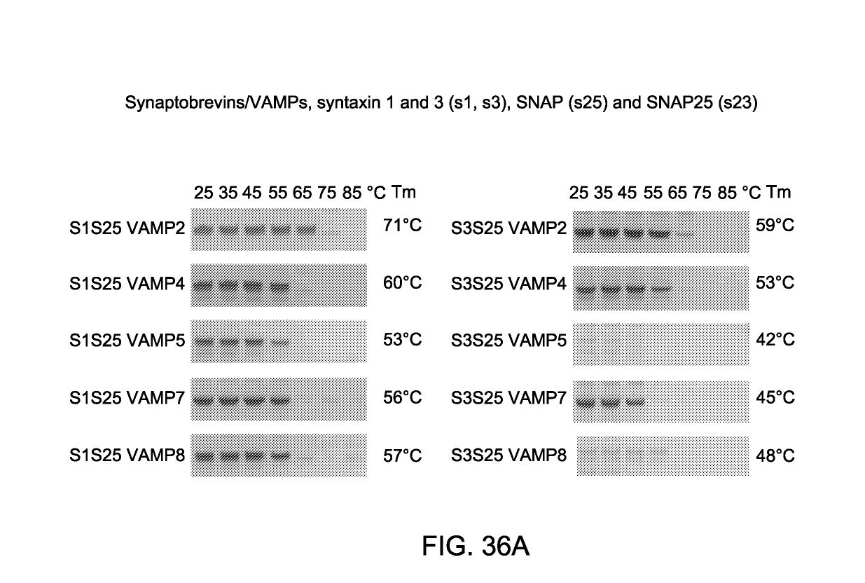

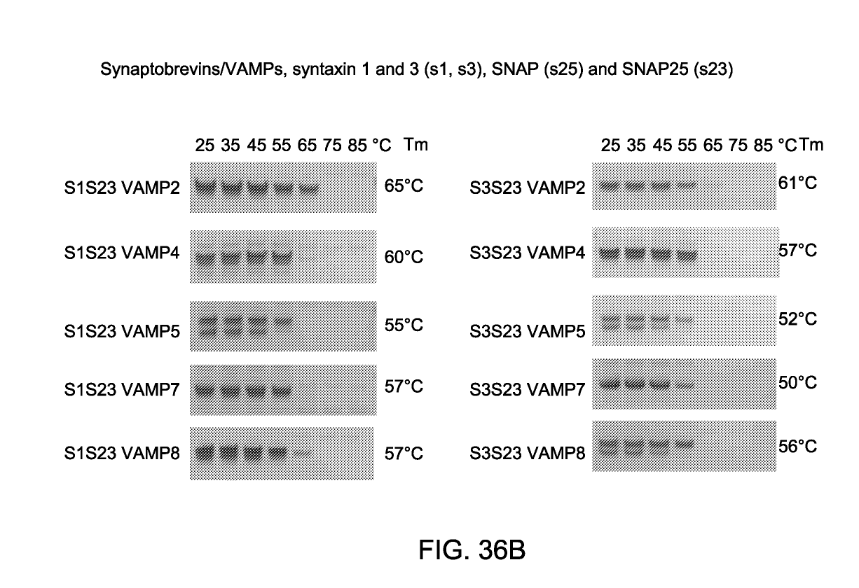

FIG. 36 is a number of SDS-gels showing formation of stable SDS-resistant complexes using a variety of different SNARE peptides.

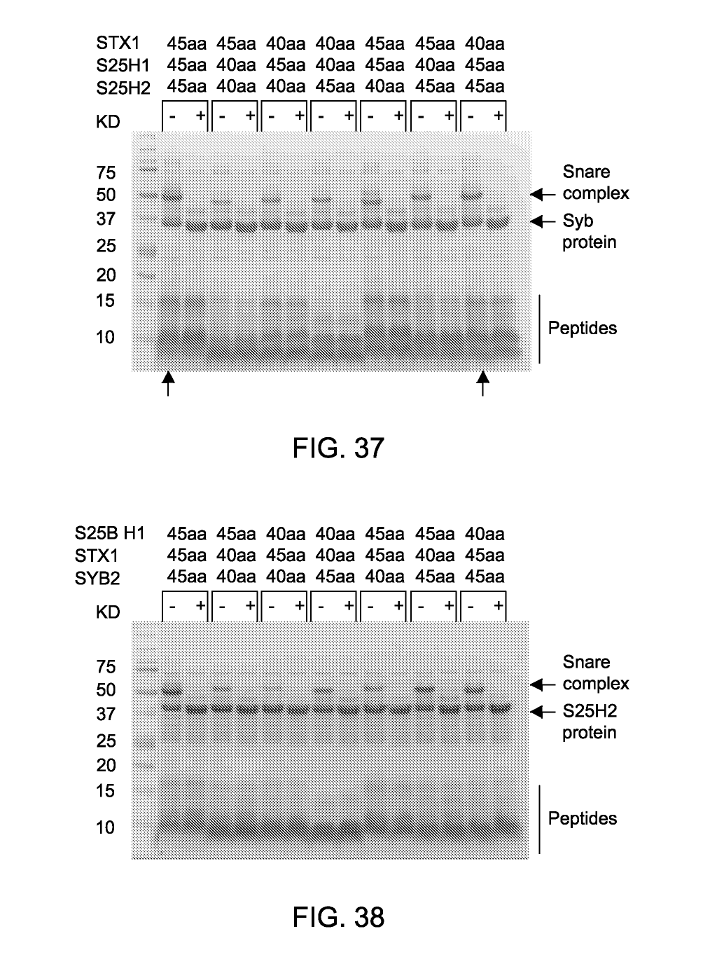

FIG. 37 is an SDS-gel showing formation of stable SDS-resistant complexes using three shortened SNARE peptides.

FIG. 38 is an SDS-gel showing formation of stable SDS-resistant complexes using three shortened SNARE peptides.

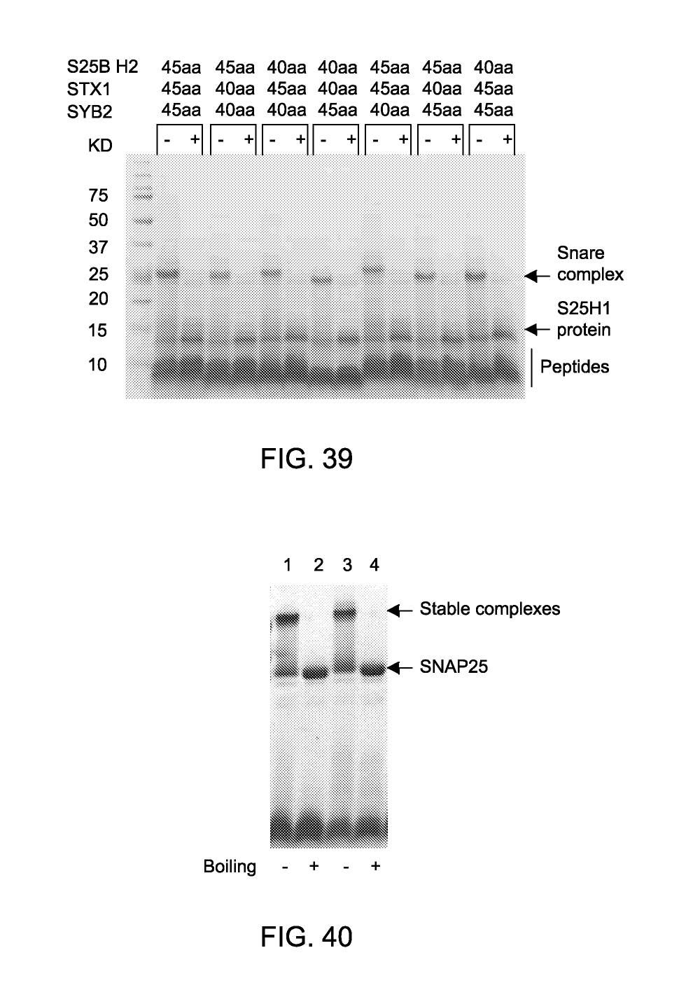

FIG. 39 is an SDS-gel showing formation of stable SDS-resistant complexes using three shortened SNARE peptides.

FIG. 40 is an SDS-gel showing formation of stable SDS-resistant complexes when a neuropeptide is complexed to one of the SNARE peptides.

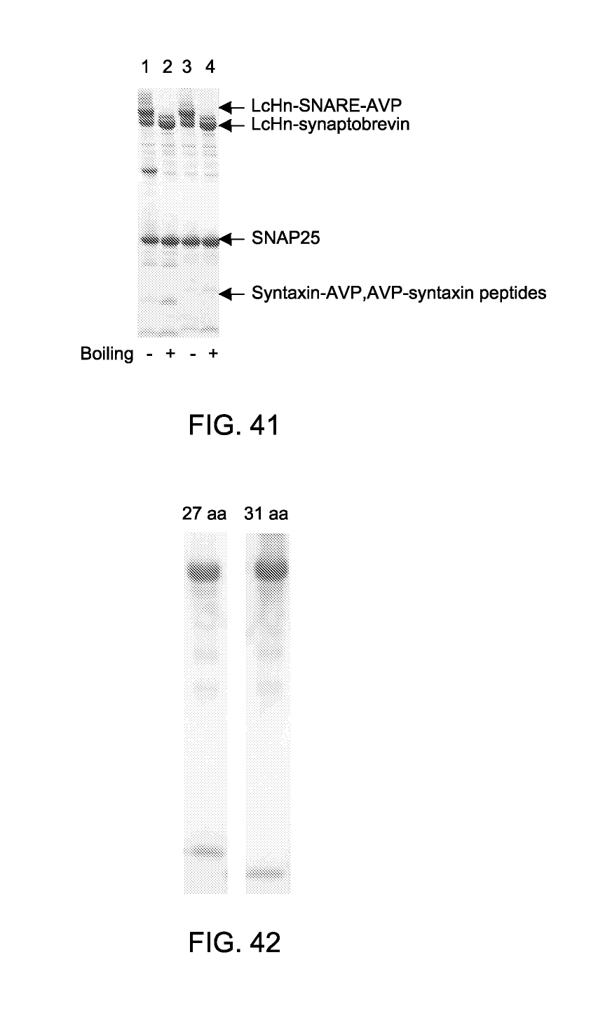

FIG. 41 is an SDS-gel showing formation of stable SDS-resistant complexes when arginine/vasopressin peptide (AVP) is complexed to the N-terminus or C-terminus of the syntaxin peptide.

FIG. 42 is an SDS-gel showing formation of stable complexes using SNARE peptides containing less than 40 amino acids.

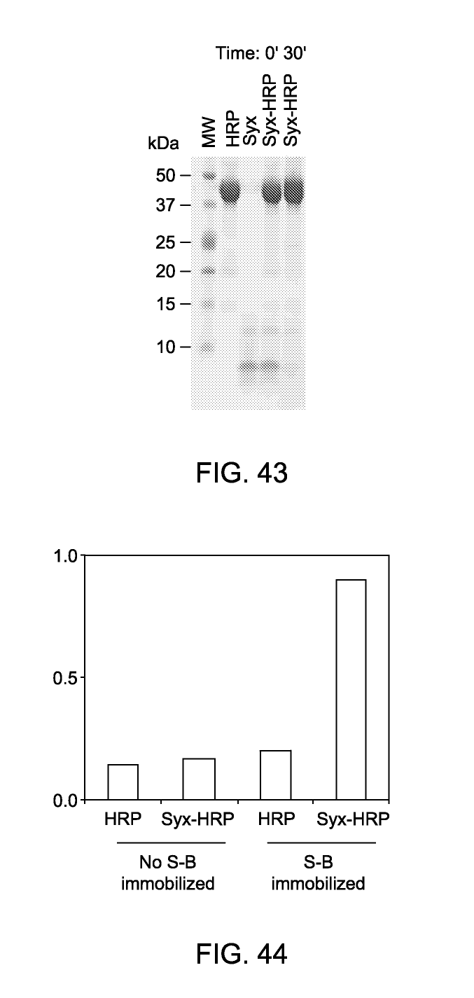

FIG. 43 is an SDS-gel showing maleimide-based cross linking of a protein to the SNARE peptides.

FIG. 44 is a chart showing absorbance at 650 nm for the TMB (3,3′,5,5′-tetramethylbenzidine) substrate.

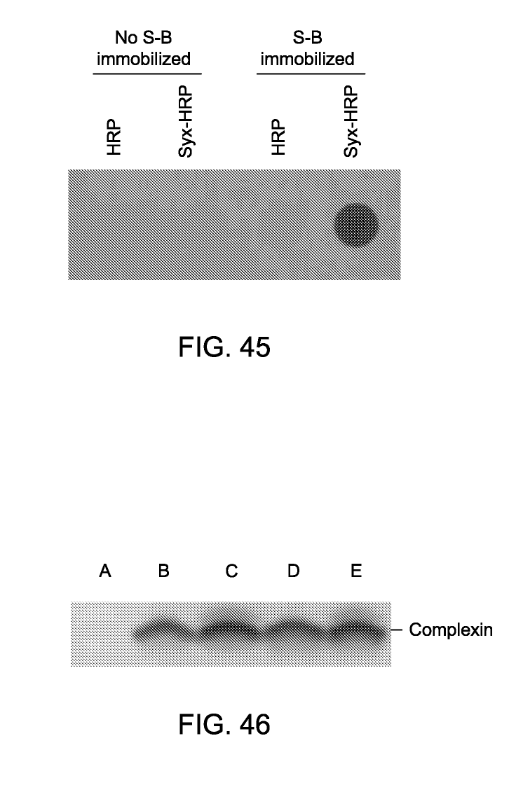

FIG. 45 is a portion of a film showing luminescence.

### EXAMPLES

Introduction

Bundle- or Linear-Shaped Scaffolds made of SNARE-Derived Polypeptides for Controlled, Irreversible, Non-Chemical Linking of Functional Units.

The inventors have developed a method for linking functional or structural units by simple mixing. Generation of well-defined, functional supramolecular architectures of nanometric size through self-assembly provide means for performing programmed engineering in life biosciences, medicine and nanotechnologies. Specifically, the invention relates to the unmet need for a controlled linking of multiple functional units in an irreversible and site-specific manner as bundles or linear multimers as depicted in FIG. 2.

The core of this new technology lies in exploitation of the unique properties of SNARE proteins for linking protein domains, and in fact any conceivable chemical entities. In nature, these proteins drive fusion of vesicles to the plasma membrane by forming a tripartite complex composed of syntaxin, SNAP-25 and synaptobrevin (also known as VAMP—vesicle-associated membrane protein). This SNARE complex is a 4-helical coiled-coil bundle comprising two helices from SNAP-25, one helix from syntaxin and one helix from synaptobrevin (FIG. 3); the length of each helix being ˜60-70 amino acids (Jahn and Scheller (2006)). This 4-helical bundle is unusually stable, even in SDS—a direct indication of the irreversible nature of SNARE assembly (Hu, K et al., 2002).

Various strategies have been employed previously for dimerization, oligomerization, multimerization of ligands and other functional groups. Among these, chemical cross-linking of small (<5 kDa) peptides (Pillai et al., 2006; Tweedle, 2006), transglutaminase-catalyzed heterodimerization (Tanaka et al., 2004) and tetrameric streptavidin binding of biotinylated ligands (Leisner et al., 2008) represent a few examples for linking functional units. In addition, coiled coils have attracted considerable interest as design templates for oligomerization in a wide range of applications including protein engineering, biotechnological, biomaterial, basic research and medicine (Engel and Kammerer, 2000; O'Shea et al., 1993; Scherr et al., 2007). Examples of useful oligomerization domains are the leucine zipper of GCN4 comprised of 33 residues which form a parallel coiled coil homodimer and 46 residue-homopentameric coiled coil COMPcc (Engel and Kammerer, 2000). The choice of a coiled coil for various applications depends on several characteristics: the length of the coiled coil polypeptides, their solubility, their ability to allow homo- or hetero-oligomerization; and the strength of the coiled coil, i.e. ability to withstand dissociation in normal and adverse conditions.

The unique properties of the SNARE coiled-coil bundle such as hetero-tetramerization and the irreversible nature of SNARE assembly have not been considered yet for exploitation. The core idea of using the SNARE bundle relates to the means of producing diagnostic/therapeutic/biotechnological protein which must carry a combination of different cargoes (fluorescent, radioactive, immune, chemical, affinity, etc.). The inventors have proposed using engineered polypeptides, based on the syntaxin, SNAP-25 and synaptobrevin proteins, for production of well-defined, organised heterotetrameric supramolecular architectures capable of self-assembly from distinct individual components. The inventors first define the minimal core for the 4- and 3-component bundles; second, the inventors describe a 2-component capture system for irreversible binding; and, third, the inventors show the usefulness of the SNARE bundle to produce linear multimers.

### Example 1

### 4-Component Bundle

The inventors show that shortened SNARE helices can be used for assembly of functional units as a 4-component bundle. Use of shortened SNARE helices is essential for attachment of chemical entities to the SNARE peptides via a synthetic route. At present, peptide synthesis is sufficiently reliable and financially feasible for ˜50 amino acids. Therefore, it would be advantageous if SNARE helices are shortened allowing attachment of further peptide sequences or other chemical entities. It is known that shortening of a single SNARE motif can lead to disruption of irreversible SNARE assembly (Hao et al., 1997). The inventors have found that (1) truncation of all four helices to 45 amino acid peptides still allows a stable tetrahelical complex, and (2) various functional groups can be added to either terminus of these peptides. This allows a simple fabrication of multivalent complexes in a bundle for a variety of uses, including affinity reagents and kits, or multivalent therapeutics (where display of an array of ligands, or multimerisation of receptors, is desired). The free ends of the 4 distinct helices can be used for attachment, by synthetic or recombinant means, of up to 8 distinct entities in desirable spatial combinations. For brevity, the inventors call the irreversible heterooligomeric protein complex—tetrahelical combinatorial scaffold (TetriCS).

The inventors synthesized TetriCS peptides containing either 40 or 45 amino acids.

40 amino acids (aa):

Rat SNAP25A Helix 1 (amino acids 28-67) with biotin:

(SEQ ID NO. 1)

STRRMLQLVEESKDAGIRTLVMLDEQGEQLDRVEEGMNHIGSGGG-
biotin

(40 amino acids SNARE sequence in bold);

Rat SNAP25A Helix 2 (amino acids 149-188) with 6-Histidine tag:

(SEQ ID NO. 2)

NLEQVSGIIGNLRHMALDMGNEIDTQNRQIDRIMEKADSNGSGGGHHHHH
H

(40 amino acids SNARE sequence in bold);

Rat Syntaxin1A (amino acids 201-240) with 6-Histidine tag and a cysteine:

(SEQ ID NO. 3)

EIIKLENSIRELHDMFMDMAMLVESQGEMIDRIEYNVEHAGSGGGHHHHH
HC

(40 amino acids syntaxin sequence in bold);

Rat Synaptobrevin-2 (amino acids 31-70) with the S-tag epitope for monoclonal antibody recognition:

(SEQ ID NO. 4)

RLQQTQAQVDEVVDIMRVNVDKVLERDQKLSELDDRADALGSKETAAAKF
ERQHMDS

(40 amino acids SNARE sequence in bold).

In parallel, the inventors tested 45 amino acid-length TetriCS peptides:

Rat SNAP25A Helix 1 (amino acids 28-72) with biotin:

(SEQ ID NO. 5)

biotin-STRRMLQLVEESKDAGIRTLVMLDEQGEQLDRVEEGMNHINQD
MKC

(45 amino acids SNARE sequence in bold);

Rat SNAP25A Helix 2 (amino acids 149-193) with a cysteine and 6-Histidine tag:

(SEQ ID NO. 6)

CNEMDENLEQVSGIIGNLRHMALDMGNEIDTQNRQIDRIMEKADSNKTRI
DGGHHHHHH

(45 amino acids SNARE sequence in bold);

Rat Syntaxin1A (amino acids 201-245) with an N-terminal antibody epitope and a cysteine:

(SEQ ID NO. 7)

Ac-AEDAEIIKLENSIRELHDMFMDMAMLVESQGEMIDRIEYNVEHAVDY
VEC

(45 amino acids SNARE sequence in bold);

Rat Synaptobrevin2 (amino acids 31-75) with an N-terminal epitope for antibody recognition:

(SEQ ID NO. 8)

MSATAATVPPAAPAGEGGPPAPPPNLTSNRRLQQTQAQVDEVVDIMRVNV
DKVLERDQKLSELDDRADALQAGAS

(45 amino acids SNARE sequence in bold).

The 40 or 45 amino acid peptides were incubated for 60 min at 20° C. and their assembly was analysed by SDS-PAGE. FIG. 4 shows that 45 amino acid Tetrics peptides were able to form the irreversible SNARE complex (panel A), while 40 amino acids peptides did not (panel B).

The functionality of the assembly was tested in pull-down experiments. The inventors used GST-tagged synaptobrevin (45 amino acid SNARE sequence, produced recombinantly in bacteria), biotin chemically linked to Helix 1 of SNAP-25 and 6-Histidine tag linked to Helix 2 of SNAP-25 as functional units. The inventors tested binding of the TetriCS assembly to glutathione beads (for GST binding) or Nickel beads (for binding the 6-Histidine tag) followed by binding of fluorescent streptavidin (for binding to biotin). FIG. 5 shows that streptavidin could bind 45 aa TetriCS attached to either glutathione or Nickel beads in a highly specific manner.

These results show that the 45aa TetriCS peptides can be used for attaching various functional groups without compromising their functional properties. Since four Tetrics peptides have eight free ends, it is possible to attach eight different groups. Thus, TetriCS allows the development of functional supramolecular devices, defined as structurally organised and functionally integrated systems built from suitably designed molecular components performing a given action (Lehn, 2007).

### Example 2

### 3-Component Bundle

In cases when less than eight groups are to be attached, it will be useful to have a simplified TetriCS assembly. Indeed, it is possible to utilise the full-length SNAP-25 molecule (amino acids 1-206) already carrying 2 SNARE helices (see FIG. 6).

The inventors therefore tested whether the 40 and 45 aa rat syntaxin 1A and synaptobrevin 2 peptides described above can form an irreversible assembly with full-length rat SNAP-25B. FIG. 7 shows that both 40 (panel A) and 45aa (panel B) peptides can assemble with SNAP25B, demonstrating that in the 3-component system, peptides of 40 aa length are sufficient for irreversible binding to full-length SNAP-25.

The inventors tested the functionality of 40 aa Tetrics assembly containing.

(i) Full-length rat SNAP25B (amino acids 1-206) with substitutions of cysteines 84, 85, 90, 92 to alanines This is known to aid expression and purification of SNAP-25 (Fasshauer et al., 1999).

(ii) Synthetic syntaxin-myc peptide (40 amino acids syntaxin sequence in bold)

(SEQ ID NO. 9)

EIIKLENSIRELHDMFMDMAMLVESQGEMIDRIEYNVEHA-GSGEQKLIS
EEDLC

(iii) Synthetic synaptobrevin-S-tag peptide (40 amino acids synaptobrevin sequence in bold)

(SEQ ID NO. 4)

RLQQTQAQVDEVVDIMRVNVDKVLERDQKLSELDDRADAL-
GSKETAAAKFERQHMDS

The functionality of the assembly was tested, following binding of GST-fused SNAP-25 to glutathione beads, by the ability of the assembled 3-component TetriCS to bind fluorescent anti-Myc antibody and fluorescent S-protein (FIG. 8). Control reactions with GST-SNAP-25 alone on beads exhibited negligible binding, whereas addition of 40 aa syntaxin and synaptobrevin peptides led to formation of 3-component TetriCS as evidenced by a robust binding of both anti-Myc antibody and S-protein to glutathione beads.

The inventors concluded that 40 aa synthetic peptides allow assembly of various tags within a single irreversible complex. The 3-component system represents a self-assembling set of 2 short polypeptides and the SNAP-25 molecule allowing an easy 6-way multifunctional tagging of proteins.

The inventors next used Surface Plasmon Resonance technique to test the ability of the 40aa 3-component TetriCS to withstand harsh treatment. The inventors chemically attached GST-tagged SNAP-25B to the Biacore CM5 chip, followed by binding of the 40 aa syntaxin and synaptobrevin peptides and then of the antibody. FIG. 9 shows that the binding of peptides is stable and allows attachment of anti-myc antibody to the surface of the chip. Addition of 0.1% SDS removed the antibody but not the peptides from the immobilised SNAP-25.

### Example 3

### 2-Component Bundle

The inventors also simplified the irreversible SNARE assembly to a 2-component system. Two-component affinity-based tools underlie all basic research and are invaluable in the development of drugs and diagnostics (Uhlen, 2008). Applications include affinity chromatography, microarray technologies, microplate-based screens and many biotechnology processes. The main factor underlying successful outcome of these applications relies on a firm, irreversible immobilization of a protein in a defined orientation on either a solid surface or three-dimensional matrix. Several recent reviews highlighted a number of disadvantages of existing immobilization technologies (Kohn, 2009; Tomizaki et al., 2005). For example, in the case of chemical protein coupling, one can achieve irreversible surface immobilization but the product may be in a non-functional state due to orientation issues and chemical modifications. In contrast, it is possible to attach a protein in a site-selective way using various tags (GST, His-tag, anti-myc and other antibody-recognizing epitopes) to corresponding surfaces (glutathione-, metal-, antibody-covered) but in all these cases immobilization is not-permanent (all existing peptide affinity tags will dissociate) and/or is very expensive (antibody-based affinity surfaces). Clearly, the ideal immobilization technique should allow both irreversible coupling and site-specific orientation of target protein and, in addition, should be considerably less expensive than current antibody-based approaches. Separately, irreversible linking of two proteins in a functional orientation becomes possible if they can be expressed with irreversibly-binding peptides.

For the 2-component system, the inventors used a de-novo designed syntaxin/synaptobrevin fusion protein together with the 2-helical SNAP-25 (see FIG. 10).

In the first embodiment, the inventors linked rat syntaxin1A SNARE motif (amino acids 195-254) with the rat synaptobrevin2 SNARE motif (aa 25-84) as illustrated in FIG. 11. The short stretch of amino acids between the syntaxin and synaptobrevin sequences (GILDSMGRLELKL (SEQ ID NO. 10), a small arrow) is due to a multiple cloning site in the hybrid plasmid.

The inventors purified the fusion protein but found that it had a tendency to aggregate via the syntaxin1 motif preventing formation of SNARE complexes with SNAP-25. Next, the inventors produced a fusion of rat syntaxin3 with synaptobrevin2. In this chimera the inventors fused the SNARE motif of rat syntaxin3 (amino acids 195-253) via a short stretch of amino acids to rat synaptobrevin2 sequence 1-84. The short stretch of amino acids between the syntaxin and synaptobrevin sequences (GILDSMGRLELKL) is due to a multiple cloning site in the hybrid plasmid allowing insertion of functional units between syntaxin3 and synaptobrevin2. When mixed with rat SNAP-25B, this syntaxin3-synaptobrevin2 fusion protein quickly assembled into an irreversible complex as illustrated in FIG. 12.

In the next chimera, the inventors used both the head domain and the SNARE motif of rat syntaxin3 (amino acids 1-260) fused via a short stretch of amino acids to rat synaptobrevin2 sequence 1-84 (see FIG. 13). The short stretch of amino acids between the syntaxin and synaptobrevin sequences (GILDSMGRLELKL (SEQ ID NO. 10)) is due to a multiple cloning site in the hybrid plasmid allowing insertion of functional units between syntaxin3 and synaptobrevin2.

It is known that the head domain of syntaxin 3 protects its SNARE motif from SNARE assembly but certain lipids can ‘open’ syntaxin for SNARE assembly (Darios and Davletov, 2006; Rickman and Davletov, 2005). Unpublished work by the inventors has shown that a mild detergent called octylglucopyranoside can also ‘open’ the syntaxin 3 molecule. Thus, the inventors can control the syntaxin SNARE motif and form a SNARE complex in a regulated manner. FIG. 14A shows one example of such controlled 2-component assembly as tested in a SDS-gel. The inventors have also found that other detergents or similar lipid compounds have the same effect, such as MEGA 8, C-HEGA 10, C-HEGA 11, HEGA 9, heptylglucopyranoside, octylglucopyranoside, nonylglucopyranoside, zwittergent 3-08, zwittergent 3-10 and zwittergent 3-12.

Surface Plasmon Resonance experiments demonstrated exquisite resistance of this 2-component assembly to various disrupting agents (FIG. 15).

As an alternative 2-component system, the inventors have produced a binary affinity reagent with one small tag in which a short syntaxin helix (<5 kDa) can bind irreversibly to a de novo-designed tri-helical SNARE fusion protein (˜27-31 kDa) represented schematically in FIG. 16A. In this chimera the inventors fused the two SNAP-25 SNARE helices (22-206 amino acids) to the SNARE motif of synaptobrevin2 sequence 1-84. The linker GSGSEQKLISEEDLG (SEQ ID NO. 11) between the SNAP-25 and synaptobrevin sequences carries a myc-tag epitope. When mixed with syntaxin 40 or 45 amino acids peptides, described earlier, the tri-helical fusion protein quickly assembled into an irreversible complex as illustrated in FIG. 16B.

These two-component systems are useful alternatives to current affinity tags (Terpe, 2003). Both tags in the binary capture systems can be expressed in bacteria and easily added, in a site-specific manner, to any protein for recombinant production—this is different from biotin/streptavidin or similar very high affinity systems (biotin can not be expressed as part of the protein). Fast capture from highly diluted solutions is now possible due to the irreversible nature of the binary affinity reagents—no other such system currently exists. When necessary, either of the tags in the binary system can be chemically linked to surfaces of beads, chips, microarray plates, and modified by chemical or recombinant introduction of functional groups.

As an example of using two-component system for purification of a protein from a bacterial extract, the inventors first immobilized to BrCN-Sepharose beads the two-helical fusion protein containing the SNARE motif of rat syntaxin3 (amino acids 195-253) fused via a short stretch of amino acids to rat synaptobrevin2 sequence 1-84 (described above). The inventors also fused an enzyme called glutathione-S-transferase (GST) to SNAP-25B and expressed the latter fusion protein in E. coli bacteria. Following disruption of bacterial membranes using 2% detergent called zwittergent 3-08, the bacterial extract was loaded onto Sepharose beads containing the syntaxin3/synaptobrevin2 fusion protein. Following washing, the beads were analysed by SDS-PAGE and Coomassie staining FIG. 17 shows that GST-SNAP-25 can bind to the Sepharose beads carrying syntaxin3/synaptobrevin2 fusion protein in a highly specific manner.

### Example 4

### Controlled Linear Polymerization Reaction

In addition to attaching multiple groups in a bundled fashion, SNARE proteins also offer a possibility of producing advanced supramolecular devices in the form of strong linear multimers of unlimited length as depicted in FIG. 18.

In such an orientation, the inventors' assembly represents a unique approach in which biomaterials are assembled molecule by molecule to produce novel linear supramolecular architectures (for overview of possible applications (Hinman et al., 2000; Lehn, 2007; Ryadnov and Woolfson, 2003; Zhang, 2003)).

In this case, the inventors used the syntaxin3-synaptobrevin2 fusion protein described above for the 2-component system (rat syntaxin 3 (amino acids 1-260) fused via a short stretch of amino acids to rat synaptobrevin2 sequence 1-84). However, instead of mixing SNAP-25 and the syntaxin3-synaptobrevin2 fusion in solution, the inventors used a solid support with a single syntaxin3 polypeptide with the head domain (amino acids 1-260 fused to GST) to initiate the polymerization reaction. The inventors first immobilized the GST-syntaxin3 molecule alone on glutathione beads via a GST tag. Then the inventors added the SNAP25B molecule (amino acids 1-206) allowing formation of syntaxin3/SNAP-25 binary complex in the presence of 0.8% octylglucopyranoside. Following washing of the beads to remove unbound SNAP-25 the inventors added the syntaxin3-synaptobrevin2 fusion protein. This process of addition of 2 building blocks was repeated as many times as necessary. The process is depicted in FIG. 19.

FIG. 20A shows the step-wise increase in the amounts of both syntaxin3-synaptobrevin2 fusion and SNAP-25 over the course of the above process. While the amount of GST-syntaxin3, used for attachment to beads, remains constant there is a gradual increase in the amounts of bound SNAP-25 and syntaxin3-synaptobrevin2 fusion protein. To show the amounts of bound material, the samples were boiled to disrupt SDS-resistant nature of the assemblies. FIG. 20B shows the increase in the molecular weight of SDS-resistant polymers in line with extra polymerization steps as the samples were not boiled prior to SDS-PAGE.

Since every step in the above process is controlled, it is possible to add at any step of the polymerization distinct SNAP-25 or syntaxin3-synaptobrevin fusion proteins with any necessary cargo in a required position. SNAP-25 and syntaxin3-synaptobrevin represent building blocks for controlled fabrication of diverse molecular structures. Applications include fabrication of functionalised nanofibers, multiple ligand microarrays, supermolecular enzyme assemblies, new electronic devices and biomaterials for use in biotechnology and medicine (for overview of possible applications (Zhang, 2003)).

It is also possible to form branches in the linear scaffold, at specific points, as depicted in FIG. 21, using syntaxin-synaptobrevin-syntaxin or SNAP-25-syntaxin fusion proteins, as described above.

Finally, the inventors explored the possibility of making linear polymers not on a surface but by simple mixing the two components in solution. The inventors utilised a modified version of syntaxin3-synaptobrevin2 fusion protein in which the linker region between the two SNARE proteins was removed. This fusion construct had syntaxin3 residues (1-253) directly fused to synaptobrevin2 residues (29-84). See FIG. 22.

The inventors mixed the fusion protein with rat SNAP-25B (aa 1-206) and analysed the 60 min reaction by SDS-gel electrophoresis. FIG. 23 shows that SNARE bundles can be multimerised in solution by simple mixing.

Therefore, the 2-component system described above also allows multimerization in solution, which presents a way to link 1 to 4 protein functional domains in a linear fashion. In addition, the protein-based fabrication of fully SDS-resistant linear polymers can be used for creation of biodegradable fibres with properties superior to silk spider multi-component assemblies or current coiled-coil nanofibers. For an overview see Hinman et al., 2000; Ryadnov and Woolfson, 2003.

Sequences of Polypeptides Used in Examples 1-4 (with an Indication of the Corresponding Figures):

Syx3(1-260)/Syb2(1-84) (FIGS. 13, 14 & 20) (syntaxin with the head domain and a linker)—SEQ ID NO. 12

Syx3(195-253)/Syb2(1-84) (FIGS. 12 & 17) (syntaxin without the head domain but with a linker)—SEQ ID NO. 13

Syx3(1-253)/Syb2(29-84) (FIG. 22) (syntaxin with the head domain and no linker)—SEQ ID NO. 14

SNAP-25B(20-206)/Syb2(1-84) (FIG. 16B)—SEQ ID NO. 15

### Example 5

### SNARE Tagging allows a Step-Wise Assembly of Botulinum Neurotoxins

Summary

Generation of defined, functional supramolecular architectures of nanometric size through controlled linking of suitable building blocks can offer new perspectives to medicine and applied technologies. Current linking strategies often rely on chemical methods which have limitations and cannot take full advantage of the recombinant technologies. Here the inventors utilised three SNARE proteins, which form a stable tetrahelical complex to drive fusion of intracellular membranes, as versatile tags for irreversible linking of recombinant and synthetic functional units. The inventors show that SNARE tagging allows step-wise production of a functional supramolecular medicinal toxin, namely botulinum neurotoxin type A commonly known as BOTOX. Fusing the receptor-binding domain with synaptobrevin SNARE motif allowed delivery of the active part of botulinum neurotoxin, tagged with SNAP25, into neurons. The data show that SNARE-tagged toxin was able to cleave its intra-neuronal molecular target and inhibit release of neurotransmitters. These results demonstrate that the SNARE tetrahelical coiled-coil allows controlled linking of various building blocks into a functional nanomachine.

Introduction

Molecular biology and the advent of recombinant production of proteins revolutionised science. The use of recombinant polypeptides, functional fragments of proteins and whole enzymes is now widespread in medicine, diagnostics, nanotechnologies, and consumer bioindustries. Despite the obvious success of recombinant technologies, protein size remains an obstacle to producing the ever more sophisticated proteins as single functional units. It is believed that combining multiple functions in supramolecular units, rather than in individual proteins, would allow us to overcome this bottleneck. Clearly, achieving such a goal of building nanofactories or nanomachines strongly depends on our ability to link various functional units on demand and with high precision. It is surprising that current efforts in this promising field still rely on linking technologies that were invented several decades ago: biotin-streptavidin pairing, antibody-epitope recognition, chemical linking through amino- and sulfohydryl groups. These approaches are often limiting due to the need of chemical modifications of recombinant proteins or complexity of antibody-based techniques. The recently-developed ‘click’ chemistry addresses some of these issues but still relies on inorganic compounds and to date has not achieved linking of recombinant proteins into a proven supramolecular assembly. Alternative approaches based on self-assembly of DNA or oligomerizing polypeptides also have their limitations in designing multifunctional recombinant assemblies. Here the inventors explored a possibility of using the SNARE (Soluble N-ethylmaleimide sensitive factor Attachment protein REceptor) protein assembly, discovered nearly two decades ago, to achieve irreversible linkage of recombinant polypeptides into a functional unit.

SNARE proteins drive fusion of cellular membranes in every eukaryotic cell by forming a heteromeric tetrahelical coiled-coil. The brain-derived SNARE complex consists of three proteins: synaptobrevin, syntaxin and SNAP25. Whereas syntaxin and synaptobrevin each contribute a single helix, SNAP25 contributes two helices to form the tetrameric coiled-coil. The four SNARE motifs are 55 amino acids long carrying eight characteristic heptade repeats. The brain SNARE complex is extraordinary in its stability exhibiting resistance to chaotropic agents, strong detergents, proteases and elevated temperatures. The inventors decided to investigate whether fusing the SNAREs to recombinant proteins would allow a controlled building of a supramolecular entity.

As an example of a multifunctional molecule the inventors focused on a botulinum neurotoxin which was described as a ‘nanomachine that unites recognition, trafficking, unfolding, translocation, refolding and catalysis’. The Botulinum NeuroToxin type A (BoNT/A) has proven to be of great medical importance due to its ability to cause a very long neuromuscular paralysis upon local injections of minute amounts (1 pM concentration) (Montecucco, C. et al. (2009)). BoNT/A is a 150 kDa protein consisting of three main modules: 50 kDa catalytic part (Light chain, Lc) which is joined via a disulphide bridge to so-called Heavy chain which in turn made of the N-terminal 50 kDa translocation part (HN) and the C-terminal 50 kDa part (Hc), the latter being responsible for recognition of neuronal gangliosides and synaptic vesicle receptor, SV2C (Mahrhold, S. et al. (2006) & Dong, M. et al. (2006)). The three main modules can be recognized as separate structural units in an X-ray model (adapted from Lacy et al. (1998), FIG. 24a). The catalytic part, when in synaptic cytosol proteolyses its intraneuronal target, SNAP25, with exquisite specificity, leading to a long-term blockade of neurotransmission (Schiavo, G. et al. (1993) & Blasi, J. et al. (1993)). The BoNT/A-mediated removal of nine amino acids from the C-terminal end of SNAP25 does not compromise stability of the SNARE assembly with syntaxin and synaptobrevin (Hayashi, T. et al. (1994)).

Results

The inventors first fused the SV2-binding part (Hc) to synaptobrevin (FIG. 24b) and tested whether this fusion can deliver quantum dots (Q-dots) into neuronal endings. The Hc-synaptobrevin fusion was able to form the SNARE complex with SNAP25 and a 52 amino acid syntaxin3 peptide labelled with biotin for binding to streptavidin-coated Q-dots. We chose to use the syntaxin3 SNARE motif rather than syntaxin1 because the latter has a tendency to homooligomerise. FIG. 24c shows that the Hc-synaptobrevin fusion was able to form an irreversible (SDS-resistant) SNARE complex, in the presence of SNAP25, even with the modified syntaxin3 motif. The Q-dots with prebound biotinylated syntaxin peptide were incubated with Hc-synaptobrevin in the presence of SNAP25 and the delivery of Q-dots into synaptic endings was assessed in cultures of hippocampal neurons obtained from mice. Q-dots carrying He-SNARE accumulated at synaptic contacts as confirmed by staining with the vesicular protein synaptophysin (FIG. 24d). This shows that the targeting part of BoNT/A is still capable of recognizing its synaptic receptor and can deliver a large cargo following recombinant fusion with a SNARE tag.

Next, the inventors prepared a fusion of the enzymatic part, translocating part and SNAP25 (FIG. 25a). We introduced a thrombin cleavage (instead of the native trypsin-sensitive site) between the enzymatic (Lc) and translocating parts (HN) facilitating the cleavage, during the isolation procedure, between these two parts which are still held together by the disulphide bond. The LcHN-SNAP25 fusion was successfully expressed in E. coli and could be purified to homogeneity. When treated with dithiothreitol this fusion separates into two parts showing the functionality of the critical disulphide bond (FIG. 25b). We then tested whether the SNARE tags will allow an assembly of the LcHN and He parts into a single entity. FIG. 25c shows that combining the two recombinant fusions, in the presence but not in the absence, of the syntaxin peptide led to emergence, within 60 minutes, of a new molecular entity LcHN-SNARE-Hc as evidenced by the SDS gel. To aid visualisation of targeting of the reassembled BoNT/A we used a fluorescein-labelled version of the syntaxin peptide and the LcHN-SNARE-Hc indeed can be visualised as a fluorescent protein (FIG. 25c).

When the LcHN-SNARE-Hc was applied to cultured hippocampal neurons, the fluorescent molecule colocalised to a significant degree with the vesicular marker synaptophysin indicating its binding to the native target of the BoNT/A (FIG. 26a). Crucially, immunoblotting of the treated neurons with anti-SNAP25 antibody demonstrated that SNAP25 undergone cleavage in the same fashion as when neurons were treated with the native BoNT/A molecule (FIG. 26b). This shows that the enzymatic part was successfully released into neuronal cytosol upon entry of the LcHN-SNARE-Hc into synaptic vesicles. To test the effect of LcHN-SNARE-Hc on neurotransmitter release we used a 96-well glutamate release assay which allows simultaneous comparison of multiple factors (Darios, F. et al. (2009)). FIG. 27a shows that LcHN-SNARE-Hc was able to inhibit calcium-and KCl-dependent release of glutamate from isolated brain nerve endings with similar dose dependency as the native BoNT/A. The degree of inhibition of the glutamate release from central synaptic ending is in good agreement with the value obtained previously (McMahon, H. T. et al. (1992)) and suggests that not all central synapses carry the SV2C receptor for BoNT/A (Dong, M. et al. (2006)). Importantly, mixing the SNARE-tagged LcHN and Hc in the absence of the linking syntaxin peptide resulted in inactive molecules, confirming that full SNARE assembly is the key factor in linking recombinant parts into a functional entity (FIG. 27b). Treatment with dithiotreitol of LcHN-SNARE-Hc inactivated the assembled toxin, indicating the functionality of the disulphide bond between Lc and HN (FIG. 27b). Finally, we tested the ability of LcHN-SNARE-Hc to paralyse muscles. We applied several concentrations of the assembled neurotoxin on isolated mouse diaphragm and tested the paralytic response of phrenic nerves. The time required to decrease the amplitude to 50% of the starting value (paralytic half-time) was determined. FIG. 27c shows that LcHN-SNARE-Hc paralysed the diaphragm muscle at subnanomolar concentrations (190 pM) within 72 min. No paralysis was observed in the absence of the linking syntaxin peptide (data not shown).

Here the inventors demonstrated that SNARE tagging allows a step-wise assembly of a medicinal toxin, BoNT/A commonly known as BOTOX (Davletov, B. et al. (2005)). Although the efficiency of the LcHN-SNARE-Hc in blocking neuromuscular junctions was less than the native BoNT/A (Mahrhold, S. et al. (2006)), this can be explained by either reduced ability of the extended toxin to reach distant active zones within long neuromuscular junctions or due to a large volume of the presynaptic ending at the diaphragm muscle leading to a compromised efficiency. However, in the context of interneuronal synaptic contacts we observed a similar efficiency between LcHN-SNARE-Hc and the native neurotoxin. Such preferential effects on the inhibition of inter-neuronal synapses but not neuromuscular junctions could be advantageous in the development of pain-inhibitory thereapeutics that avoid muscle-paralysing side effects. The implications of our observations are many with some being listed here. First, it is now possible to express an active form of a multimodular medicinal toxin in bacteria in a safe way; in fact, our use of a ‘locking’ peptide allows an additional safety feature. It is also possible to utilize the SNARE-tagged Hc part to deliver imaging agents and future therapeutics by tagging them with SNARE counterparts (Binz, T et al. (2009)). Further, it is possible to oligomerise Hc part for eliciting a stronger immune response when producing anti-botulinum serum (Webb, R. P. et al. (2007)). The SNARE tagging of the LcHN part will also allow an easy-retargeting of the active portion of BoNT/A to specific neuroendocrine cells (Dolly, J. O. et al. (2009)). Here one can target neuropeptide or growth factor receptors by making corresponding SNARE-tagged ligands. Such SNARE tagging can allow a convenient combinatorial mixing of various functional units with the aim of finding the most beneficial combination(s) to silence specific subsets of neurons (Foster, K. A. (2009)).

While the inventors used BoNT/A as an example of a sophisticated ‘nanomachine’, it is clear that SNARE tagging can be used in building, in a highly controlled manner, many further supramolecular assemblies. Generation of well-defined, functional supramolecular architectures of nanometric size through controlled linking of suitable building blocks is believed to offer new perspectives for many fields (Lehn, J. M. (2007)). The relatively short SNARE motifs allow combination of both recombinantly-produced polypeptides and inorganic molecules as evidenced by incorporation of biotin and fluorescein upon assembly of the botulinum neurotoxin. The greatest advantage of the SNARE coiled-coil is its heterotetrameric nature allowing linking of up to eight distinct functionalities. This potential has yet to be exploited in future medicine and applied technologies.

Methods

Plasmids and protein reactions. All proteins were expressed in BL21 strain of E. coli as glutathione-S-transferase (GST) fusions. The plasmid for expression of LcHN-SNAP25 was generated as follows: cDNA of BoNT/A Lc (amino acids 1-449) was amplified by PCR and inserted into SmaI and EcoRI restrictions sites in the pGEX-KG vector (Guan, K. L. et al. (1991)). The codon optimised cDNA of BoNT/A translocation domain FIN (amino acids 450-872, from ATG Biosynthetics, Germany) was inserted at the 3′ end of light chain. The thrombin cleavage site (amino acids LVPRGS (SEQ ID NO. 16)) was inserted between the light chain and the translocation domain of BoNT/A. Finally, the cDNA of rat SNAP25B (aa 1-206) was inserted at the 3′ end of FIN. The plasmid allowing expression of Hc-Syb was generated as follows: cDNA of rat synaptobrevin (amino acids 25-84) was amplified by PCR and inserted into pGEX-KG vector between BamHI and EcoRI sites. The cDNA of the BoNT/A heavy chain (amino acids 876-1296) was amplified by PCR and inserted at the 3′ end of synaptobrevin. A peptide of the syntaxin3 SNARE motif (amino acids 200-250) was synthesized chemically with either biotin or fluorescein (Peptide Synthetics, UK). Proteins fused to GST were purified on glutathione Sepharose beads (GE Healthcare, USA) and eluted from beads in 20 mM Hepes, pH 7.3, 100 mM NaCl using thrombin. The supramolecular complexes were assembled by mixing the SNARE-tagged proteins with the syntaxin peptide for 1 hr at 22 ° C.

Neuronal imaging and immunoblotting. Mouse anti-synaptophysin antibody (clone 7.2) was from Synaptic Systems, and mouse anti-SNAP25 antibody (clone SMI81) was from Sternberger Monoclonals. Streptavidin-conjugated Q-dots 525 were from Invitrogen. Primary cultures of hippocampal neurons were prepared as described (Darios, F. et al. (2009)) and used after 7-10 days in vitro. Neurons were exposed to SNARE-tagged, or native toxin, for 2 hours, fixed with 4% PFA and then immunostained with anti-synaptophysin antibody. Fluorescence was observed on a Radiance Confocal system (Zeiss/Bio-Rad; Hemel Hempstead, Herts., U.K.) linked to a Nikon Eclipse fluorescence microscope. Alternatively, neurons were incubated for 20 hours with assembled toxin or native BoNT/A, lysed in 60 mM Tris, pH 6.8, 2 mM MgCL₂, 2% SDS, benzonase (Novagen, 250 U/ml) and then SNAP25 cleavage was analysed by immunoblotting using an anti-SNAP25 antibody.

Blockade of neurotransmitter release. Rat brain synaptosomes were freshly isolated as described (Darios, F. et al. (2009)). Synaptosomes (0.5 mg/ml of proteins) were incubated in buffer A (in mM, 132 NaCl, 5 KCl, 20 HEPES, 1.2 NaH₂PO₄, 1.3 MgCl₂, 0.15 Na₂EGTA, 1 MgSO₄, 5 NaHCO₃, 10 D-glucose) with LcHN-SNARE-Hc at indicated concentrations for 1 hr at 37° C. An equal volume of buffer A containing glutamate dehydrogenase (15 units/ml, Sigma) and 3 mM NADP (Sigma) was added for 10 min. Glutamate release was induced by addition of KCl (35 mM) in the presence of 2 mM CaCl₂ and monitored by following fluorescence (Exc. 340 nm, Em. 460 nm) (Darios, F. et al. (2009) & McMahon, H. T. et al. (1992)). The Phrenic Nerve hemidiaphragm assay was performed as described previously (Mahrhold, S. et al. (2006)). Mouse phrenic nerves were derived from Naval Medical Research Institute (NMRI) mice. The phrenic nerve was continuously stimulated at 5-25 mA with a frequency of 1 Hz, 0.1 ms pulse duration. Isometric contractions were transformed using a force transducer and recorded with VitroDat Online software (FMI GmbH). The time required to decrease the amplitude to 50% of the starting value (paralytic half-time) was determined.

Additional Information

Botulinum neurotoxins are the most potent toxins designed by nature. These toxins are produced by Clostridium bacteria to cause long-lasting paralysis and death. Over the last 30 years, some members of the botulinum family, e.g. botulinum neurotoxin type A (BoNT/A) also known as BOTOX, have been successfully exploited for medicinal and cosmetic purposes. These toxins silence neuromuscular junctions and also can block neurotransmitter release from many types of neurons. Practically every part of the human body, with the exception of the brain, can be treated by BOTOX. Since the paralysis of neuromuscular junctions is reversible, the sustained relaxation of muscles requires repeat injections every three to four months. BoNT/A can block innervation of not only striated muscles but also of smooth muscles. Furthermore, the cholinergic junctions of the autonomous nervous system that control sweating, salivation and other types of secretion are as sensitive to BOTOX as are the neuromuscular junctions. Therefore, BOTOX-based treatments have recently expanded to include a dazzling array of nearly a hundred conditions from dystonias to gastrointestinal and urinary disorders.

The effectiveness of BoNT/A in clinical medicine has led to increasing interest in other members of the botulinum family. Comparative studies have demonstrated that BoNT/A has the longest paralysing effect among the seven immunologically distinct serotypes of BoNTs (A-G), thus underpinning the usefulness of specifically BoNT/A in the treatment of neurological disorders. All BoNTs are synthesised by the bacteria as single polypeptide chains with a molecular mass of 150 kDa. Following bacterial death and lysis, the toxins are ‘nicked’ by bacterial proteases to yield the 50 kDa light and the 100 kDa heavy chains that are kept together by a disulphide bond. The two chains, still linked through the disulphide bond, traverse the intestinal epithelial cells by transcytosis, enter the bloodstream and eventually bind to peripheral cholinergic nerve terminals.

The extreme toxicity of BoNTs indicates that the peripheral nerve endings carry molecules that can serve as BoNTs' high-affinity receptors. Indeed, several synaptic vesicle proteins have been shown to act as receptors for BoNTs. While the heavy chains are responsible for BoNTs' binding to nerve terminals, the light chains are potent endopeptidases that attack the vesicle fusion machinery and therefore have to get inside the nerve terminal. BoNTs accomplish this task by hijacking the vesicle endocytosis route. As the pH of the recycling vesicle's interior drops, the BoNTs undergo major conformational changes. This enables the translocating part (known as HN) of the heavy chains to form putative channels across the vesicular membrane through which the partially unfolded light chains slip into the cytosol. On entry into the cytosol, reduction of the disulphide bond frees the light chain from the heavy chain.

BoNT light chains are potent endopeptidases that attack a number of isoforms of the three SNARE proteins that mediate vesicle fusion and therefore neurotransmitter release. It is now known that BoNT/A and BoNT/E proteolyse SNAP-25, while BoNTs B, D, F and G cleave VAMP on the synaptic vesicles. SNAP-25 shortened by only nine amino acids by BoNT/A retains its ability to interact with the plasma membrane syntaxin and vesicular synaptobrevin but cannot mediate the normal vesicle fusion process. Further information about botulinum neurotoxins (BoNTs) can be found in: Davletov, B., Bajohrs, M. and Binz, T., Trends Neurosci 28, 446-452 (2005); Johnson, E. A. (1999) Annu Rev Microbiol 53, 551-575; Jankovic, J. (2004) J Neurol Neurosurg Psychiatry 75 (7), 951-957; Aoki, K. R. and Guyer, B. (2001) Eur J Neurol 8 Suppl 5, 21-29; Simpson, L. L. (2004) Annu Rev Pharmacol Toxicol 44, 167-193; Dolly, O. (2003) Headache 43 Suppl 1, S16-24; and Montecucco, C. and Schiavo, G. (1993) Trends Biochem Sci 18 (9), 324-327. The complete sequence information for BoNT/A (BOTOX) was published in Binz, T. et al. (1990) J Biol Chem 265 (16), 9153-9158.

Retargeting Strategies:

To date the benefits of BoNTs have been restricted to treatments of neuromuscular conditions and disorders of the automonous nervous system. BoNTs, however, can also block neurotransmitter release in central neurons, making it possible to exploit them in experimental neuroscience and in future neurology dealing with higher brain functions. Studies on brain slices, cultured neurons and synaptosomes have demonstrated that BoNTs can stop the neurotransmitter release of not only acetylcholine but also glutamate, glycine, noradrenaline, dopamine, serotonin, ATP and various neuropeptides (Ashton et al. (1988), Capogna, M. et al. (1997), Sanchez-Prieto, J. et al. (1987), Verderio, C. et al. (2004), Luvisetto, S. et al. (2004), and Costantin, L. et al. (2005)). The light chains of BoNTs are naturally delivered by their partner heavy chains, but alternative means of delivery, such as liposomes or recombinant fusion constructs, are also effective (de Paiva, A. et al. (1990), Chaddock, J. A. et al. (2004), and Duggan, M. J. et al. (2002)). Recombinant chimeras of lectins with the BoNT/A light chain recently allowed delivery of the latter into nociceptive afferents or dorsal root ganglia (Chaddock, J. A. et al. (2004)). Importantly, the delivery efficiency can be easily tracked if the light chain is fused to a GFP fluorescent tag, allowing marking of the silenced cells. The ability of the heavy chains of BoNTs to target synapses and transport their light chains into the nerve terminal offers another tool that can be utilised in neurobiology (Goodnough, M. C. et al. (2002) and Bade, S. et al. (2004)). Delivery of various molecules, especially enzymes, using BoNT heavy chains may be feasible for manipulation of synapse physiology. Indeed, it has recently been demonstrated that BoNT/D can deliver recombinantly attached enzymes into the nerve terminals (Bade, S. et al. (2004)).

The inventors have shown that it is possible to obtain a functional botulinum neurotoxin by recombining the toxin from two parts:

The light chain (LC) with the translocation domain (FIN) where the latter carries on its C-terminus a SNARE tag (syntaxin, SNAP-25 or synaptobrevin); and

Receptor-binding part of the heavy chain (HC part) which carries on its N-terminus a SNARE tag (syntaxin, SNAP-25 or synaptobrevin).

SNARE tags allow irreversible linking of functional units.

These two parts (LCHN and HC) can be produced separately in a protein-producing bacterial strain without health risks. Each part can be purified in a safe way. When the two parts are mixed they produce within 1 hour an active neurotoxin which can cleave its molecular target SNAP-25 in exposed neurons and also block neurotransmitter release from nerve endings.

Since the LCHN part with a SNARE tag is functional (i.e. SNAP-25 neuronal cleavage and a blockade of neurotransmitter release is observed), it is possible to direct this active part to specific neurons or endocrine cells by adding a ligand with a SNARE tag for irreversible assembly of LCHN/ligand moiety. One example is somatostatin peptide linked to a SNARE tag. Binding of the ligand to the intended cells can result in the transport of LCHN into the cell with subsequent release of the light chain and therefore SNAP-25 damage with subsequent halt in neurotransmitter or hormone release.

Since the HC part with a SNARE tag is functional (i.e. observation of a blockade of neurotransmitter release after attachment of LCHN/SNARE tagged), it is possible to use the SNARE tagged HC part for delivery of other enzymatic or imaging moieties into neurons. It is also possible to use other receptor-binding compounds (e.g. somatostatin neuropeptide) with a SNARE tag to deliver drugs, imaging reagents, etc. into specific cells that carry necessary receptors.

The sequences of LCHN tagged with SNARE motifs and the sequences of HC tagged with SNARE motifs are given below:

cDNA encoding BoNT/A Light chain (aa 1-449), translocation domain (aa 449-872) and SNARE proteins were fused by molecular biology techniques. A thrombin site LVPR-GS (SEQ ID NO. 17) was inserted between the light chain (LC) and the translocation domain (FIN) of BoNT/A to mimic natural nicking of toxin by trypsin (represented by a “-” in sequence above). Proteins fused to GST were purified as described. Proteins were eluted in 20 mM Hepes, pH 7.3, 100 mM NaCl from beads using thrombin. In the case of Synaptobrevin 2 (25-84) BoNT/A Hc (876-1296), 0.8% octylglucoside was present in the elution buffer. After elution we obtained proteins with the following sequences:

SEQ ID NO. 18

LcHN-SNAP25: BoNT/A Lc(1-449) Thrombin
HN(449-872)-SNAP25B (C to A)

SEQ ID NO. 19

LcHN-Syx: BoNT/A Lc(1-449) Thrombin HN(449-872)-
Syx3(195-253)

SEQ ID NO. 20

LcHN-Syb: BoNT/A Lc(1-449) Thrombin HN(449-872)-
Syb2 (1-96; WWK-AAA)

SEQ ID NO. 21

Syb-HcA: Syb2 (25-84) BoNT/A Hc (876-1296)

SEQ ID NO. 22

Syb HcD: Syb2 (25-84) BoNT/D Hc (863-1276)

SEQ ID NO. 23

Nanolock HcA: Syx 3 (195-253) Syb 2 (1-84) BoNT/A
Hc (876-1296)

SEQ ID NO. 24

SNAP25B (C toA)

SEQ ID NO. 25

Syntaxin 3 (195-253)

In addition to the recombinant proteins obtained by bacterial production (all above), synthetic peptides were also used:

Syx3 peptide (45aa, 52 aa-FITC)
Somatostatin-synaptobrevin peptide
Somatostatin-syntaxin peptide
somatostatin peptide

SEQ ID NO. 26

Ac-RLQQTQAQVDEVVDIMRVNVDKVLERDQKLSELDDRADAL-Ahx-
Ahx-AGCKNFFWKTFTSC-OH

To link the catalytic part to receptor-binding domain part, the inventors mixed for 1 hr the active part fused to one SNARE protein (LcHN-Syx, LcHN SNAP25 or LcHN-Syb) with the receptor binding part containing a second SNARE protein. The assembly was locked by the addition of the third SNARE partner. Examples of combinations are given in the table:

| Catalytic Domain | Locking protein | Receptor-binding part |
|---|---|---|
| LcHN-Syx3 | SNAP25 | Syb-HcA |
| LcHN-SNAP25 | Syx3 (45aa) | Syb-HcA |
| LcHN-SNAP25 | Syx3 (52aa-FITC) | Syb-HcA |
| LcHN-SNAP25 | Syx3 (195-253) | Syb-HcA |
| LcHN-SNAP25 | — | Nanolock-HcA |
| LcHN-Syx3 | SNAP25 | Syb-Somatostatin |
| LcHN-Syb | SNAP25 | Syx peptide-AVP |

### Example 6

The following example shows the possibility of obtaining stable SDS-resistant complexes by cutting the N-terminal end of SNARE motifs:

Full SNARE motif of Synaptobrevin 2 (from N-terminal to C-terminal):

(SEQ ID NO. 27)

RLQQTQAQVDEVVDIMRVNVDKVLERDQKLSELDDRADALQAGASQFETS
AAKLA

The following synthetic synaptobrevin peptides were tested which were N-terminal truncations of the SNARE motif of Synaptobrevin 2:

(SEQ ID NO. 28)

1) FITC-Ahx-
AQVDEVVDIMRVNVDKVLERDQKLSELDDRADALQAGASQFETSAAKLA
(49 amino acids);

(SEQ ID NO. 29)

2) FITC-Ahx-DIMRVNVDKVLERDQKLSELDDRADALQAGASQFETSA
AKLA (42 amino acids);

(SEQ ID NO. 30)

3) FITC-Ahx-DKVLERDQKLSELDDRADALQAGASQFETSAAKLA
(35 amino acids);
and

(SEQ ID NO. 31)

4) FITC-Ahx-KLSELDDRADALQAGASQFETSAAKLA
(27 amino acids).

FIG. 33 shows that the synaptobrevin SNARE motif can be reduced to 42 amino acids and still forms an SDS-resistant complex with syntaxin1 (stx1) and SNAP25 (S25). The 35 amino acid synaptobrevin SNARE motif also forms a complex but it ‘melts’ during gel-electrophoresis.

### Example 7

It is also possible to reduce the SNARE motif from both the N- and C-termini as exemplified by shortening of the syntaxin SNARE motif In addition, replacement of internal residues is permissive for SNARE assembly for tailored applications.

Full SNARE motif of Syntaxin 1:

(SEQ ID NO. 32)

EIIKLENSIRELHDMFMDMAMLVESQGEMIDRIEYNVEHAVDYVERAVSD
TKKA

The following synthetic syntaxin peptides were tested:

(SEQ ID NO. 33)

1) FITC-Ahx-EIIRLENSIRELHDMFMDMAMLVESQGEMIDRIEYNVE
HAVDYVERA-Ahx-KK-NH2 (47 amino acids);

(SEQ ID NO. 34)

2) Ac-HHHHHH-Ahx-
EIIRLENSIRELHDMFMDMAMLVESQGEMIDRIEYNVEHAVDYVEC
(45 amino acids);

(SEQ ID NO. 35)

3) EIIKLENSIRELHDMFMDMAMLVESQGEMIDRIEYNVEHAGSGGGHH
HHHHC (40 amino acids);

(SEQ ID NO. 36)

4) Flu-GGEIIRLENSIRELHDMFMDMAMLVESQGEMID
(31 amino acids);

(SEQ ID NO. 37)

5) EIIRLENSIRELHDMFMDMAMLVESTGEMIDRIEYNVEHA-NH2
(40 amino acids);
and

(SEQ ID NO. 38)

6) Bio-NSIRELHDMFMDMAMLVESQGEMIDRIEYNVEHAVDYVEC
(39 amino acids).

The results are shown in FIG. 34. Syntaxin 1 with 40 amino acids is sufficient for complex formation. The 39 amino acid peptide, shortened from both termini and carrying biotin on the N-terminus and additional cysteine useful for further modifications, forms a stable complex as shown in the SDS gel. Note, it is possible to replace the syntaxin internal lysine (K204) with arginine and still retain strong complexation with SNAP25 and synaptobrevin. Such replacements are advantageous if lysines should be added on the N- or C-termini for peptide modifications or immobilization on surfaces.

It is also possible to replace all internal methionines with non-oxidizable norleucines to obtain a stable version of syntaxin SNARE peptide:

(SEQ ID NO. 39)

Wild-type (Met)-FITC-Ahx-
EIIKLENSIRELHDMFMDMAMLVESQGEMIDRIEYNVEHAVDYVE-NH2

(SEQ ID NO. 40)

Norleucine peptide-FITC-Ahx-
EIIKLENSIRELHDnFnDnAnLVESQGEnIDRIEYNVEHAVDYVE-NH2

The SDS-gel of FIG. 35 shows that comparable assemblies can be made from SNAP-25, synaptobrevin 45 aa peptides, and the two syntaxin peptides above.

### Example 8

Many combinations of SNARE motifs can be employed for the complexing system. Examples of stable, SDS-resistant complexes and their melting temperatures (Tm) are provided for SNARE complexes made of recombinant syntaxins 1 and 3, SNAP23 and SNAP25, and VAMPs 2, 4, 5, 7 and 8, as visualised in the SDS gels shown in FIG. 36.

The strongest complexes (higher melting temperature, Tm) are obtained with syntaxin1, VAMP2 and either SNAP25 or SNAP23.

The amino acid sequences of additional SNARE isoforms produced in bacteria and used here are:

SNAP-23—SEQ ID NO. 41

VAMP4—SEQ ID NO. 42

VAMP5—SEQ ID NO. 43

VAMP7—SEQ ID NO. 44

VAMP8—SEQ ID NO. 45

### Example 9A

The complexing system affords uniting of 3 separate shortened polypeptide SNARE motifs.

Recombinant synaptobrevin (VAMP2) fused to glutathione-S-transferase can be united with 40 and 45 amino acid syntaxin1 and SNAP25 peptides. SNAP25 peptides are designated as helix 1 (S25H1) and helix 2 (S25H2).

Syx1 45 aa: SEQ ID NO. 46;

Syx1 40 aa: SEQ ID NO. 47;

S25H1 45 aa: SEQ ID NO. 48;

S25H1 40 aa: SEQ ID NO. 49;

S25H2 45 aa: SEQ ID NO. 50; and

S25H2 40 aa: SEQ ID NO. 51.

The results are shown in FIG. 37 in which the lanes marked with + contain samples which were boiled in SDS and the lanes marked with − contain samples which were not boiled in SDS.

### Example 9B

Recombinant S25H2 protein can be united with 40 and 45 amino acid syntaxin1, VAMP2 and S25H1 peptides.

S25H1 45 aa: SEQ ID NO. 48;

S25H1 40 aa: SEQ ID NO. 49;

Syx1 45 aa: SEQ ID NO. 46;

Syx1 40 aa: SEQ ID NO. 47;

Syb2 45 aa: SEQ ID NO. 52; and

Syb2 40 aa: SEQ ID NO. 53.

The results are shown in FIG. 38 in which the lanes marked with + contain samples which were boiled in SDS and the lanes marked with − contain samples which were not boiled in SDS.

### Example 9C

Recombinant S25H1 protein can be united with 40 and 45 amino acid syntaxin1, VAMP2 and S25H2 peptides.

S25H2 45 aa: SEQ ID NO. 50;

S25H2 40 aa: SEQ ID NO. 51;

Syx1 45 aa: SEQ ID NO. 46;

Syx1 40 aa: SEQ ID NO. 47;

Syb2 45 aa: SEQ ID NO. 52; and

Syb2 40 aa: SEQ ID NO. 53.

The results are shown in FIG. 39 in which the lanes marked with + contain samples which were boiled in SDS and the lanes marked with − contain samples which were not boiled in SDS.

### Example 10

Complexing of neuropeptides can be accomplished in various combinations:

Examples are provided using the following peptides:

Syntaxintag-somatostatin:

(SEQ ID NO. 54)

Ac-EIIKLENSIRELHDMFMDMAMLVESQGEMIDRIEYNVEHA-Ahx-
Ahx-AGCKNFFWKTFTSC-OH
Brevintag-somatostatin:

(SEQ ID NO. 55)

Ac-RLQQTQAQVDEVVDIMRVNVDKVLERDQKLSELDDRADAL-Ahx-
Ahx-AGCKNFFWKTFTSC-OH
Syntaxintag-SubstanceP:

(SEQ ID NO. 56)

Ac-EIIKLENSIRELHDMFMDMAMLVESQGEMIDRIEYNVEHAVDYVE-
Ahx-Ahx-RPKPQQFFGLM-NH2
Brevintag-SubstanceP:

(SEQ ID NO. 57)

Ac-RLQQTQAQVDEVVDIMRVNVDKVLERDQKLSELDDRADALQAGAS-
Ahx-Ahx-RPKPQQFFGLM-NH2

Combining these peptides affords alternative stable combinations as exemplified in FIG. 40. In FIG. 40, lane 1 and 2: Brevintag-SubstanceP was mixed with Syntaxintag-somatostatin in the presence of SNAP25. The stable complex contains both substanceP and somatostatin (heterodimeric peptide). Lane 3 and 4: Brevintag-somatostatin was mixed with Syntaxintag-somatostatin in the presence of SNAP25. The stable complex contains two somatostatins (homodimeric peptide). Note, lanes 1 and 3 show the stable complexes obtained using tagged neuropeptides.

### Example 11

Neuropeptides can be united with other functional groups, e.g. botulinum neurotoxin parts, in different orientations which could affect binding to cell surface receptors and translocation of peptides. In this example the following sequences were used:

45 aa Syntaxin motif-arginine/vasopressin
peptide (AVP)

(SEQ ID NO. 58)

Ac-AEIIKLENSIRELHDMFMDMAMLVESQGEMIDRIEYNVEHAVDYVE
R-Ahx-Ahx-CYFQNCPRG-NH2
arginine/vasopressin peptide-45 aa Syntaxin motif

(SEQ ID NO. 59)

CYFQNCPRG-Ahx-Ahx-
AEIIKLENSIRELHDMFMDMAMLVESQGEMIDRIEYNVEHAVDYVE-NH2

Combining these peptides with LcHn part of botulinum toxin A affords alternative stable combinations visualised in the SDS-gel of FIG. 41.

Lanes 1 and 2: syntaxin-AVP was incubated with LcHn-brevin in the presence of SNAP-25 for 60 min. The stable complex contains LcHn-SNARE-AVP (AVP is on the C-terminal end of the SNARE linkers).

Lanes 3 and 4: AVP-syntaxin was incubated with LcHn-brevin in the presence of SNAP-25 for 60 min. The stable complex contains LcHn-AVP-SNARE (AVP is on the N-terminal end of the SNARE linkers).

### Example 12

Peptides shorter than 40 can still form stable complexes (but not SDS-resistant). Sequences used:

biotin-Ahx-EIIRLENSIRELHDMFMDMAMLVESQG-NH2 (SEQ ID
NO. 60) - 27 aa syntaxin1 peptide
Biotin-Ahx-EIIKLENSIRELHDMFMDMAMLVESQGEMID-NH2
(SEQ ID NO. 61) - 31 aa syntaxin peptide

Binding of the peptides was observed in a pull-down experiment using GST-SNAP25linkerSynaptobrevin protein (trihelical construct) immobilized on glutathione-beads. Binding was for 30 min at 25 degrees C., followed by extensive washing in buffer A (20 mM HEPES, 100 mM NaCl). Protein and peptides were visualised on SDS-gels (FIG. 42). Biotinylated peptides bound to beads are seen at the bottom of the SDS-gel.

### Example 13

SNARE peptides can be chemically cross-linked to proteins when the proteins cannot be expressed recombinantly. The peptides retain their ability to form the SNARE complex and the modified proteins retain their activity. Sequences used:

45aa syntaxin1 peptide

(SEQ ID NO. 62)

Ac-AEDAEIIKLENSIRELHDMFMDMAMLVESQGEMIDRIEYNVEHAVDY
VEC

The above peptide was cross-linked to Maleimide-Horse Radish Peroxidase (Sigma-Aldrich Co.).

FIG. 43 is an SDS-gel showing maleimide-based cross-linking

The cross-linked syntaxin-HRP can bind to GST-trihelix (Snap25-brevin, S-B) which was immobilized on glutathione beads. The results are shown in FIGS. 44 and 45. FIG. 44 shows adsorbance at 650 nm of the TMB substrate. FIG. 45 shows luminescence of luminal visualized on film. Note, control glutathione beads without GST-trihelix (S-B) show only background binding of syntaxin-HRP.

### Example 14

A 29 amino acid complexin 1 peptide can interact with SNARE assemblies. This peptide can be used for purification following its immobilization or as an additional carrier in SNARE-based assemblies.

Complexin Peptide:

(SEQ ID NO. 63)

Ac-ERKAKYAKMEAEREVMRQGIRDKYGIKKGSGSGGIKVAV-NH2

FIG. 46 is an SDS-gel showing pull down of the complexin peptide by the following proteins immobilized on Ni2+ beads.

A. Full length HIS-SNAP25 (no SNARE complex)

B. Full length HIS-SNAP25+Full length Syntaxin1+Full length Synaptobrevin2

C. Full length HIS-SNAP25+Full length Syntaxin1+Synaptobrevin2 45aa peptide

D. Full length HIS-SNAP25+Syntaxin1 45aa peptide+Full length Synaptobrevin2

E. Full length HIS-SNAP25+Syntaxin1 45aa peptide+Synaptobrevin2 45aa peptide

In particular, lane E represents a product where one recombinant protein binds three synthetic peptides one of which is the complexin peptide.

### Example 15

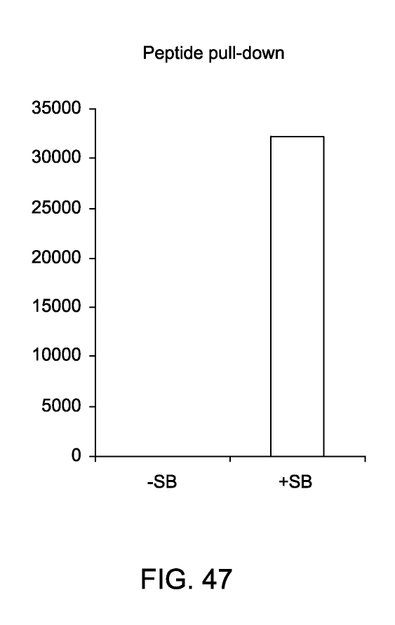

SNARE assembly can be accomplished in the presence of blood serum. Certain applications will require de novo interaction of SNARE-based medicines following their injection into blood. The 45 amino acid syntaxin1 peptide with FITC fluorescent tag was tested for binding to GST-trihelical protein (SNAP-25 linked to synaptobrevin, SB protein) immobilised on glutathione beads in the presence of 100% calf serum. The results are shown in FIG. 47. Note, the presence of the trihelical protein on beads results in pull-down of the syntaxin fluorescent peptide in the presence of serum. The vertical axis represents relative fluorescence units.

### REFERENCES

Ashton, A. C. and Dolly, J. O. (1988) J Neurochem 50 (6), 1808-1816.

Bade, S. et al. (2004) J Neurochem 91 (6), 1461-1472.

Besteiro (2006). BMC Genomics 7:250.

Bhattacharya, S., Stewart, B. A., Niemeyer, B. A., Burgess, R. W., McCabe, B. D., Lin, P., Boulianne, G., O'Kane, C. J., and Schwarz, T. L. (2002). Proceedings of the National Academy of Sciences of the United States of America 99, 13867-13872.

Binz, T. and Rummel, A., J Neurochem 109 (6), 1584 (2009).

Blasi, J. et al., Nature 365 (6442), 160 (1993).

Bowen, M. E., Weninger, K., Ernst, J., Chu, S., and Brunger, A. T. (2005). Biophysical journal 89, 690-702.

Capogna, M. et al. (1997) J Neurosci 17 (19), 7190-7202.

Chaddock, J. A. et al. (2004) Mov Disord 19 Suppl 8, S42-47.

Costantin, L. et al. (2005) J Neurosci 25 (8), 1943-1951.

Darios, F. et al., Neuron 62 (5), 683 (2009).

Davletov, B., Bajohrs, M., and Binz, T., Trends Neurosci 28 (8), 446 (2005).

de Paiva, A. and Dolly, J. O. (1990) FEBS Lett 277 (1-2), 171-174.

Dolly, J. O. et al., Curr Opin Pharmacol 9 (3), 326 (2009).

Dong, M. et al., Science 312 (5773), 592 (2006).

Duggan, M. J. et al. (2002) J Biol Chem 277 (38), 34846-34852.

Engel, J., and Kammerer, R. A. (2000). Matrix Biol 19, 283-288.

Fasshauer, D., Antonin, W., Margittai, M., Pabst, S., and Jahn, R. (1999). J Biol Chem 274, 15440-15446.

Fasshauer, D., and Margittai, M. (2004). The Journal of biological chemistry 279, 7613-7621.

Foster, K. A., Toxicon 54 (5), 587 (2009).

Goodnough, M. C. et al. (2002) FEBS Lett 513 (2-3), 163-168.

Guan, K. L. and Dixon, J. E., Anal Biochem 192 (2), 262 (1991)

Hao, J. C., Salem, N., Peng, X. R., Kelly, R. B., and Bennett, M. K. (1997). J Neurosci 17, 1596-1603.

Hayashi, T. et al., EMBO J 13 (21), 5051 (1994).

Hinman, M. B., Jones, J. A., and Lewis, R. V. (2000). 18, 374-379.

Hu, K., Carroll, J., Fedorovich, S., Rickman, C., Sukhodub, A., and Davletov, B. (2002). Nature 415, 646-650.

Hu et al. (2002) Action of Complexin on SNARE Complex. J. Biol. Chem., 277, 41652-41656Jahn R and Scheller R H. Nat Rev Mol Cell Biol 2006 September; 7(9): 631-43.

Jahn R, and Scheller R H. Nat Rev Mol Cell Biol. 2006 September; 7(9):631-43

Kammerer, R. A., Kostrewa, D., Progias, P., Honnappa, S., Avila, D., Lustig, A., Winkler, F. K., Pieters, J., and Steinmetz, M. O. (2005). Proc Natl Acad Sci USA 102, 13891-13896.

Karlsson and Falt (1997). Journal of Immunological Methods Volume 200, Issues 1-2, Pages 121-133

Kasai and Akagawa (2001) Journal of Cell Science 114, 3115-3124.

Kohn, M. (2009). J Pept Sci 15, 393-397.

Laage et al. (2000). J Biol Chem. 275:17481-7.

Lacy, D. B. et al., Nat Struct Biol 5 (10), 898 (1998).

Lauer, J. M., Dalal, S., Marz, K. E., Nonet, M. L., and Hanson, P. I. (2006). The Journal of biological chemistry 281, 14823-14832.

Lehn, J. M., Chem Soc Rev 36 (2), 151 (2007)

Leisner, C., Loeth, N., Lamberth, K., Justesen, S., Sylvester-Hvid, C., Schmidt, E. G., Claesson, M., Buus, S., and Stryhn, A. (2008). PLoS ONE 3, e1678.

O'Shea, E. K., Lumb, K. J., and Kim, P. S. (1993). Curr Biol 3, 658-667.

Luvisetto, S. et al. (2004) Behav Pharmacol 15 (3), 233-240.

Mahrhold, S. et al., FEBS Lett 580 (8), 2011 (2006).

McMahon, H. T. et al., J Biol Chem 267 (30), 21338 (1992).

Montecucco, C. et al., Toxicon 54 (5), 561 (2009).

Pillai, R., Marinelli, E. R., and Swenson, R. E. (2006). Biopolymers 84, 576-585.

Pobbati, A. V., Stein, A., and Fasshauer, D. (2006). Science 313, 673-676.

Rickman and Davletov (2005) Chemistry and Biology May 12(5):545-53.

Rickman C, Meunier F A, Binz T, Davletov B (2004). J. Biol. Chem. 2004 Jan. 2; 279(1):644-51.

Rossi G et al. (1997). J Biol Chem.; 272 16610-7.

Ryadnov, M. G., and Woolfson, D. N. (2003). Nature materials 2, 329-332.

Sanchez-Prieto, J. et al. (1987) Eur J Biochem 165 (3), 675-681.

Scherr, N., Honnappa, S., Kunz, G., Mueller, P., Jayachandran, R., Winkler, F., Pieters, J., and Steinmetz, M. O. (2007). Proceedings of the National Academy of Sciences of the United States of America 104, 12151-12156.

Schiavo, G. et al., FEBS Lett 335 (1), 99 (1993).

Sieber et al. (2006) Biophys J. 90(8): 2843-2851.

Sutton, R. B., Fasshauer, D., Jahn, R., and Brunger, A. T. (1998). Nature 395, 347-353.

Tanaka, T., Kamiya, N., and Nagamune, T. (2004). Bioconjug Chem 15, 491-497.

Terpe, K. (2003). Applied microbiology and biotechnology 60, 523-533.

Tomizaki, K. Y., Usui, K., and Mihara, H. (2005). Chembiochem 6, 782-799.

Tweedle, M. F. (2006). Contrast media & molecular imaging 1, 2-9.

Uhlen, M. (2008). Biotechniques 44, 649-654.

Verderio, C. et al. (2004) Neuron 41 (4), 599-610.

Webb, R. P. et al., Vaccine 25 (21), 4273 (2007).

Yang, B., Gonzalez, L., Jr., Prekeris, R., Steegmaier, M., Advani, R. J., and Scheller, R. H. (1999). The Journal of biological chemistry 274, 5649-5653.

Zhang, S. (2003). Nat Biotechnol 21, 1171-1178.

We claim:

1. A complexing system for forming a molecular scaffold, the system comprising:
(a) two polypeptide helices derived from a SNAP protein;
(b) one polypeptide helix derived from syntaxin;
(c) one polypeptide helix derived from synaptobrevin or a homolog thereof; and
(d) one or more cargo moieties attached to the polypeptide helices,
wherein the four polypeptide helices can form a stable SNARE complex, and wherein the polypeptide helix derived from syntaxin is joined to the polypeptide helix derived from synaptobrevin or a homolog thereof.

2. A complexing system for forming a molecular scaffold, the system comprising:
(a) two polypeptide helices derived from a SNAP protein;
(b) one polypeptide helix derived from syntaxin;
(c) one polypeptide helix derived from synaptobrevin or a homolog thereof; and
(d) one or more cargo moieties attached to the polypeptide helices,
wherein the four polypeptide helices can form a stable SNARE complex, and wherein the polypeptide helices are joined together to form two helix containing components.

3. The system of claim 1, wherein two of the helices are joined together so that both helices can assemble into the same stable SNARE complex.

4. The system of claim 3, wherein the other two helices are joined together so that both helices can assemble into the same stable SNARE complex.

5. The system of claim 2, wherein three of the helices are joined together so that all three helices can assemble into the same stable SNARE complex.

6. The system of claim 5, wherein the two polypeptide helices derived from the SNAP protein and: either the polypeptide helix derived from syntaxin; or the polypeptide helix derived from synaptobrevin or a homolog thereof, are joined together.

7. The system of claim 1 or claim 2, wherein one of the helices is immobilised on a substrate.

8. The system of claim 1 or claim 2, wherein the SNAP protein is SNAP-25.

9. The system of claim 1 or claim 2, wherein the polypeptide helix of (c) is derived from synaptobrevin.

10. The system of claim 1 or claim 2, wherein each helix is at least about 40 amino acids in length.

11. The system of claim 1 or claim 2, wherein the sequence of each of the polypeptide helices has at least about 80% identity with the sequence of the protein or a portion of the protein from which the polypeptide helix is derived.

12. The system of claim 1 or claim 2 comprising a cargo moiety which comprises the light chain of a botulinum toxin or a functional portion thereof and the translocation portion of the heavy chain of a botulinum toxin.

13. The system of claim 1 or claim 2 comprising a cargo moiety which comprises the receptor binding portion of the heavy chain of a botulinum toxin, or a somatostatin peptide or functional portion thereof.

14. The system of claim 1 or claim 2 comprising a first and a second cargo moiety which are attached to separate helices or helix containing components, and wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin or a somatostatin peptide or functional portion thereof.

15. The system of claim 14, wherein the second cargo moiety comprises the receptor binding portion of the heavy chain of a botulinum toxin.

16. The system of claim 1 or claim 2, further comprising one polypeptide helix derived from complexin which can bind to the SNARE complex.

17. The system of claim 1 or claim 2 further comprising a detergent.

18. The system of claim 17, wherein the detergent is selected from MEGA 8, C-HEGA 10, C-HEGA 11, HEGA 9, heptylglucopyranoside, octylglucopyranoside, nonylglucopyranoside, zwittergent 3-08, zwittergent 3-10 and zwittergent 3-12.

19. The system of claim 17, wherein the detergent is octylglucopyranoside.

20. The system of claim 1 or claim 2, wherein two of the helices are joined together so that both helices cannot assemble into the same SNARE complex.

21. The system of claim 1 or claim 2, wherein two of the helices are joined together and wherein the system further comprises a single polypeptide helix which is derived from the same protein as one of the two helices which are joined together.

22. The system of claim 21, wherein the single polypeptide helix is immobilised on a substrate.

23. A multimer produced by the system of claim 20.

24. An apparatus having a stable SNARE complex immobilised thereon, the SNARE complex comprising:
(a) two polypeptide helices derived from a SNAP protein;
(b) one polypeptide helix derived from syntaxin;
(c) one polypeptide helix derived from synaptobrevin or a homolog thereof; and
(d) one or more cargo moieties attached to the polypeptide helices,
wherein the apparatus is selected from an array, a substrate, a microfluidic device, an SPR instrument, a QCM instrument, a mass spectrometer, an electrophoresis instrument, a chromatography column, a scanning probe microscope, and a calorimetry instrument.

25. A method of forming a SNARE complex carrying one or more cargo moiety, the method comprising:
binding together two polypeptide helices derived from a SNAP protein, one polypeptide helix derived from syntaxin, and one polypeptide helix derived from synaptobrevin or a homolog thereof to form a stable SNARE complex, wherein one or more cargo moieties is attached to the polypeptide helices, and wherein:
(i) the polypeptide helix derived from syntaxin is joined to the polypeptide helix derived from synaptobrevin or a homolog thereof; or
(ii) the polypeptide helices are joined together to form two helix containing components.

26. A component for forming a molecular scaffold, the component comprising a polypeptide helix derived from syntaxin joined to a polypeptide helix derived from synaptobrevin or a homolog thereof and wherein the two joined helices can form part of a stable SNARE complex.

27. The component of claim 26 comprising a sequence selected from SEQ ID NOs. 12, 13, 14 and 73.

28. A component for forming a molecular scaffold comprising two polypeptide helices derived from a SNAP protein and either a polypeptide helix derived from syntaxin or a polypeptide helix derived from synaptobrevin, wherein the three helices are joined together to form a tri-helical component and wherein the three joined helices can form part of a stable SNARE complex.

29. The component of claim 28 comprising the sequence of SEQ ID NO. 15.

30. A kit comprising a component comprising a polypeptide helix derived from syntaxin joined to a polypeptide helix derived from synaptobrevin or a homolog thereof and wherein the two joined helices can form part of a stable SNARE complex.

31. The kit of claim 30, further comprising two polypeptide helices derived from a SNAP protein which can form a stable SNARE complex with the syntaxin/synaptobrevin derived helices.

32. A kit comprising a component comprising two polypeptide helices derived from a SNAP protein and either a polypeptide helix derived from syntaxin or a polypeptide helix derived from synaptobrevin, wherein the three helices are joined together to form a tri-helical component and wherein the three joined helices can form part of a stable SNARE complex.

33. The kit of claim 32, further comprising a single polypeptide helix derived from the fourth SNARE protein being either synaptobrevin or a homolog thereof, or syntaxin, wherein the single polypeptide helix can form a stable SNARE complex with the tri-helical component.

34. A complexing system for forming a molecular scaffold, the system comprising:
(a) two polypeptide helices derived from a SNAP protein;
(b) one polypeptide helix derived from syntaxin;
(c) one polypeptide helix derived from synaptobrevin or a homolog thereof; and
(d) one or more cargo moieties attached to the polypeptide helices,
wherein the four polypeptide helices can form a stable SNARE complex and wherein the SNARE complex is formed in the presence of a detergent.

35. The complexing system of claim 34, wherein the complexing system does not contain a Munc18 protein.

36. A multimer comprising a plurality of stable SNARE complexes joined together, wherein each SNARE complex comprises:
(a) two polypeptide helices derived from a SNAP protein;
(b) one polypeptide helix derived from syntaxin; and
(c) one polypeptide helix derived from synaptobrevin or a homolog thereof,
wherein a helix from one SNARE complex is joined to a helix from another SNARE complex to join the SNARE complexes together, and wherein one or more cargo moiety is attached to the polypeptide helices.

37. The multimer of claim 36, further comprising a branch in which three helices from one SNARE complex are attached to a helix in three different SNARE complexes.

38. A method of producing a multimer, the method comprising the following steps:
(a) providing a first polypeptide helix derived from a first SNARE helix;
(b) binding a second and a third polypeptide helix to the first polypeptide helix to form a tri-helical complex, wherein the second and third polypeptide helices are derived from a second and a third SNARE helix;
(c) binding a fourth polypeptide helix to the tri-helical bundle to form a stable SNARE complex, wherein the fourth polypeptide helix is derived from a fourth SNARE helix, and wherein the fourth polypeptide helix is joined to a fifth polypeptide helix derived from a first SNARE helix; and
(d) repeating steps 2) and 3) to form a multimer,
wherein one or more cargo moiety is attached to the polypeptide helices.

39. The method of claim 38, wherein the identities of the second, third, fourth and fifth polypeptide helices are maintained in the repeated steps.

40. The method of claim 38, wherein the second and third polypeptide helices are joined together so that they can assemble together in the same SNARE complex.

41. The method of claim 38, wherein the first polypeptide helix is immobilised on a substrate.

42. The method of claim 38, further comprising a step of washing after each binding step to remove any unbound helices.

43. The method of claim 38, wherein a branch is introduced into the multimer by using a sixth polypeptide helix derived from a first SNARE helix, wherein the sixth polypeptide helix is attached to one of the second, third, fourth or fifth polypeptide helices.

44. A multimer produced by the method of claim 38.

45. A complexing system for forming a binary compound comprising two cargo moieties, the complexing system comprising:
two polypeptide helices derived from a SNAP protein;
one polypeptide helix derived from syntaxin; and
one polypeptide helix derived from synaptobrevin or a homolog thereof,
wherein the four polypeptide helices can form a stable SNARE complex, wherein a first cargo moiety is attached to a first helix and a second cargo moiety is attached to a second separate helix, and wherein formation of the SNARE complex causes formation of the binary compound.

46. The complexing system of claim 45, wherein the binary compound is a toxin.

47. The complexing system of claim 46, wherein the toxin is selected from botulinum toxin, diptheria toxin, tetanus toxin and ricin.

48. The complexing system of claim 47, wherein the toxin is botulinum toxin.

49. The complexing system of claim 48 comprising:
two polypeptide helices derived from a SNAP protein;
one polypeptide helix derived from syntaxin; and
one polypeptide helix derived from synaptobrevin or a homolog thereof,
wherein the four polypeptide helices can form a stable SNARE complex, wherein a first cargo moiety is attached to a first helix and a second cargo moiety is attached to a second separate helix, and wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin.

50. The complexing system of claim 47, wherein the toxin is selected from diptheria toxin and ricin.

51. A method of treating a disease or condition which is alleviated by the inhibition of neural synapses, the method comprising the administration of an effective amount of a composition comprising the system of claim 48 or 49 to a subject.

52. A method of forming a SNARE complex to form a binary compound comprising two cargo moieties, the method comprising:
binding together two polypeptide helices derived from a SNAP protein, one polypeptide helix derived from syntaxin, and one polypeptide helix derived from synaptobrevin or a homolog thereof to form a stable SNARE complex, wherein a first cargo moiety is attached to a first helix and a second cargo moiety is attached to a second separate helix, and wherein formation of the SNARE complex causes formation of the binary compound.

53. The method of claim 52, wherein the binary compound is a toxin.

54. The method of claim 53, wherein the toxin is selected from botulinum toxin, diptheria toxin, tetanus toxin and ricin.

55. The method of claim 54, wherein the toxin is botulinum toxin.

56. The method of claim 55 to form a botulinum toxin, the method comprising:
binding together two polypeptide helices derived from a SNAP protein, one polypeptide helix derived from syntaxin, and one polypeptide helix derived from synaptobrevin or a homolog thereof to form a stable SNARE complex, wherein a first cargo moiety is attached to a first helix and a second cargo moiety is attached to a second separate helix, wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin.

57. A component for forming a botulinum toxin, the component comprising: a polypeptide helix derived from: a SNAP protein; syntaxin; or synaptobrevin or a homolog thereof, wherein the polypeptide helix is attached to a cargo moiety comprising the light chain of a botulinum toxin or a functional portion thereof and the translocation portion of the heavy chain of a botulinum toxin.

58. A component for forming a botulinum toxin, the component comprising: a polypeptide helix derived from: a SNAP protein; syntaxin; or synaptobrevin or a homolog thereof, wherein the polypeptide helix is attached to a cargo moiety comprising the receptor binding portion of the heavy chain of a botulinum toxin.

59. A kit comprising two polypeptide helices derived from a SNAP protein; one polypeptide helix derived from syntaxin; and one polypeptide helix derived from synaptobrevin or a homolog thereof, wherein the four polypeptide helices can form a stable SNARE complex, wherein a first cargo moiety is attached to a first helix and a second cargo moiety is attached to a second separate helix, and wherein the first cargo moiety comprises the light chain of a botulinum toxin or a functional portion thereof and the translocation portion of the heavy chain of a botulinum toxin and the second cargo moiety comprises the receptor binding portion of the heavy chain of a botulinum toxin.

60. A complexing system for forming a molecular scaffold, the system comprising:
(a) two polypeptide helices derived from a SNAP protein;
(b) one polypeptide helix derived from syntaxin;
(c) one polypeptide helix derived from synaptobrevin or a homolog thereof; and
(d) one or more cargo moieties attached to the polypeptide helices,
wherein the four polypeptide helices can form a stable SNARE complex, and wherein at least two of the polypeptide helices are less than 50 amino acids in length.

61. A method of forming a SNARE complex carrying one or more cargo moiety, the method comprising:
binding together two polypeptide helices derived from a SNAP protein, one polypeptide helix derived from syntaxin, and one polypeptide helix derived from synaptobrevin or a homolog thereof to form a stable SNARE complex, wherein one or more cargo moiety is attached to the polypeptide helices, and wherein at least two of the polypeptide helices are less than 50 amino acids in length.


SEQUENCE LISTING (77 sequences)

SEQ ID NO: 1 - protein, 45 aa, Artificial sequence
  Synthetic construct
STRRMLQLVEESKDAGIRTLVMLDEQGEQLDRVEEGMNHIGSGGG

SEQ ID NO: 2 - protein, 51 aa, Artificial sequence
  Synthetic construct
NLEQVSGIIGNLRHMALDMGNEIDTQNRQIDRIMEKADSNGSGGGHHHHHH

SEQ ID NO: 3 - protein, 52 aa, Artificial sequence
  Synthetic construct
EIIKLENSIRELHDMFMDMAMLVESQGEMIDRIEYNVEHAGSGGGHHHHHHC

SEQ ID NO: 4 - protein, 57 aa, Artificial sequence
  Synthetic construct
RLQQTQAQVDEVVDIMRVNVDKVLERDQKLSELDDRADALGSKETAAAKFERQHMDS

SEQ ID NO: 5 - protein, 46 aa, Artificial sequence
  Synthetic construct
STRRMLQLVEESKDAGIRTLVMLDEQGEQLDRVEEGMNHINQDMKC

SEQ ID NO: 6 - protein, 59 aa, Artificial sequence
  Synthetic construct
CNEMDENLEQVSGIIGNLRHMALDMGNEIDTQNRQIDRIMEKADSNKTRIDGGHHHHHH

SEQ ID NO: 7 - protein, 50 aa, Artificial sequence
  Synthetic construct
AEDAEIIKLENSIRELHDMFMDMAMLVESQGEMIDRIEYNVEHAVDYVEC

SEQ ID NO: 8 - protein, 75 aa, Artificial sequence
  Synthetic construct
MSATAATVPPAAPAGEGGPPAPPPNLTSNRRLQQTQAQVDEVVDIMRVNVDKVLERDQKLSELDDRADALQAGAS

SEQ ID NO: 9 - protein, 54 aa, Artificial sequence
  Synthetic construct
EIIKLENSIRELHDMFMDMAMLVESQGEMIDRIEYNVEHAGSGEQKLISEEDLC

SEQ ID NO: 10 - protein, 13 aa, Artificial sequence
  Synthetic construct
GILDSMGRLELKL

SEQ ID NO: 11 - protein, 15 aa, Artificial sequence
  Synthetic construct
GSGSEQKLISEEDLG

SEQ ID NO: 12 - protein, 359 aa, Artificial sequence
  Synthetic construct
GSMKDRLEQLKAKQLTQDDDTDEVEIAIDNTAFMDEFFSEIEETRLNIDKISEHVEEAKKLYSIILSAPIPEPKTKDDLEQLTTEIKKRANNVRNKLKSMEKHIEEDEVRSSADLRIRKSQHSVLSRKFVEVMTKYNEAQVDFRERSKGRIQRQLEITGKKTTDEELEEMLESGNPAIFTSGIIDSQISKQALSEIEGRHKDIVRLESSIKELHDMFMDIAMLVENQGEMLDNIELNVMHTVDHVEKARDETKRAMKYQGQAGILDSMGRLELKLMSATAATVPPAAPAGEGGPPAPPPNLTSNRRLQQTQAQVDEVVDIMRVNVDKVLERDQKLSELDDRADALQAGASQFETSAAKL

SEQ ID NO: 13 - protein, 158 aa, Artificial sequence
  Synthetic construct
GSEGRHKDIVRLESSIKELHDMFMDIAMLVENQGEMLDNIELNVMHTVDHVEKARDEAKRAGILDSMGRLELKLMSATAATVPPAAPAGEGGPPAPPPNLTSNRRLQQTQAQVDEVVDIMRVNVDKVLERDQKLSELDDRADALQAGASQFETSAAKL

SEQ ID NO: 14 - protein, 312 aa, Artificial sequence
  Synthetic construct
GSMKDRLEQLKAKQLTQDDDTDEVEIAIDNTAFMDEFFSEIEETRLNIDKISEHVEEAKKLYSIILSAPIPEPKTKDDLEQLTTEIKKRANNVRNKLKSMEKHIEEDEVRSSADLRIRKSQHSVLSRKFVEVMTKYNEAQVDFRERSKGRIQRQLEITGKKTTDEELEEMLESGNPAIFTSGIIDSQISKQALSEIEGRHKDIVRLESSIKELHDMFMDIAMLVENQGEMLDNIELNVMHTVDHVEKARDEAKRAGNRRLQQTQAQVDEVVDIMRVNVDKVLERDQKLSELDDRADALQAGASQFETSAAKL

SEQ ID NO: 15 - protein, 283 aa, Artificial sequence
  Synthetic construct
GSADESLESTRRMLQLVEESKDAGIRTLVMLDEQGEQLERIEEGMDQINKDMKEAEKNLTDLGKFAGLAVAPANKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSGSEQKLISEEDLGMSATAATVPPAAPAGEGGPPAPPPNLTSNRRLQQTQAQVDEVVDIMRVNVDKVLERDQKLSELDDRADALQAGASQFETSAAKL

SEQ ID NO: 16 - protein, 6 aa, Artificial sequence
  Synthetic construct
LVPRGS

SEQ ID NO: 17 - protein, 6 aa, Artificial sequence
  Synthetic construct
LVPRGS

SEQ ID NO: 18 - protein, 1095 aa, Artificial sequence
  Synthetic construct
GSPGMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSMAEDADMRNELEEMQRRADQLADESLESTRRMLQLVEESKDAGIRTLVMLDEQGEQLERIEEGMDQINKDMKEAEKNLTDLGKFAGLAVAPANKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 19 - protein, 949 aa, Artificial sequence
  Synthetic construct
GSPGMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSEGRHKDIVRLESSIKELHDMFMDIAMLVENQGEMLDNIELNVMHTVDHVEKARDETKRAG

SEQ ID NO: 20 - protein, 988 aa, Artificial sequence
  Synthetic construct
GSPGMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKAKSLVPRGSQALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIRSPEFMSATAATVPPAAPAGEGGPPAPPPNLTSNRRLQQTQAQVDEVVDIMRVNVDKVLERDQKLSELDDRADALQAGASQFETSAAKLKRKYAAANLKMM

SEQ ID NO: 21 - protein, 73 aa, Artificial sequence
  Synthetic construct
GSNLTSNRRLQQTQAQVDEVVDIMRVNVDKVLERDQKLSELDDRADALQAGASQFETSAAKLGILDSMGINTE

SEQ ID NO: 22 - protein, 491 aa, Artificial sequence
  Synthetic construct
GSNLTSNRRLQQTQAQVDEVVDIMRVNVDKVLERDQKLSELDDRADALQAGASQFETSAAKLGILDSMASINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVEVGHHHHHH

SEQ ID NO: 23 - protein, 588 aa, Artificial sequence
  Synthetic construct
GSEGRHKDIVRLESSIKELHDMFMDIAMLVENQGEMLDNIELNVMHTVDHVEKARDETKRAGILDSMGRLELKLMSATAATVPPAAPAGEGGPPAPPPNLTSNRRLQQTQAQVDEVVDIMRVNVDKVLERDQKLSELDDRADALQAGASQFETSAAKLGILDSMGINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 24 - protein, 208 aa, Artificial sequence
  Synthetic construct
GSMAEDADMRNELEEMQRRADQLADESLESTRRMLQLVEESKDAGIRTLVMLDEQGEQLERIEEGMDQINKDMKEAEKNLTDLGKFAGLAVAPANKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 25 - protein, 62 aa, Artificial sequence
  Synthetic construct
GSEGRHKDIVRLESSIKELHDMFMDIAMLVENQGEMLDNIELNVMHTVDHVEKARDETKRAG

SEQ ID NO: 26 - protein, 56 aa, Artificial sequence
  Synthetic construct
RLQQTQAQVDEVVDIMRVNVDKVLERDQKLSELDDRADALXXAGCKNFFWKTFTSC

SEQ ID NO: 27 - protein, 55 aa, Artificial sequence
  Synthetic construct
RLQQTQAQVDEVVDIMRVNVDKVLERDQKLSELDDRADALQAGASQFETSAAKLA

SEQ ID NO: 28 - protein, 49 aa, Artificial sequence
  Synthetic construct
AQVDEVVDIMRVNVDKVLERDQKLSELDDRADALQAGASQFETSAAKLA

SEQ ID NO: 29 - protein, 42 aa, Artificial sequence
  Synthetic construct
DIMRVNVDKVLERDQKLSELDDRADALQAGASQFETSAAKLA

SEQ ID NO: 30 - protein, 35 aa, Artificial sequence
  Synthetic construct
DKVLERDQKLSELDDRADALQAGASQFETSAAKLA

SEQ ID NO: 31 - protein, 27 aa, Artificial sequence
  Synthetic construct
KLSELDDRADALQAGASQFETSAAKLA

SEQ ID NO: 32 - protein, 54 aa, Artificial sequence
  Synthetic construct
EIIKLENSIRELHDMFMDMAMLVESQGEMIDRIEYNVEHAVDYVERAVSDTKKA

SEQ ID NO: 33 - protein, 50 aa, Artificial sequence
  Synthetic construct
EIIRLENSIRELHDMFMDMAMLVESQGEMIDRIEYNVEHAVDYVERAXKK

SEQ ID NO: 34 - protein, 53 aa, Artificial sequence
  Synthetic construct
HHHHHHXEIIRLENSIRELHDMFMDMAMLVESQGEMIDRIEYNVEHAVDYVEC

SEQ ID NO: 35 - protein, 52 aa, Artificial sequence; identical to SEQ ID NO: 3
  Synthetic construct

SEQ ID NO: 36 - protein, 33 aa, Artificial sequence
  Synthetic construct
GGEIIRLENSIRELHDMFMDMAMLVESQGEMID

SEQ ID NO: 37 - protein, 40 aa, Artificial sequence
  Synthetic construct
EIIRLENSIRELHDMFMDMAMLVESTGEMIDRIEYNVEHA

SEQ ID NO: 38 - protein, 40 aa, Artificial sequence
  Synthetic construct
NSIRELHDMFMDMAMLVESQGEMIDRIEYNVEHAVDYVEC

SEQ ID NO: 39 - protein, 45 aa, Artificial sequence
  Synthetic construct
EIIKLENSIRELHDMFMDMAMLVESQGEMIDRIEYNVEHAVDYVE

SEQ ID NO: 40 - protein, 45 aa, Artificial sequence
  Synthetic construct
EIIKLENSIRELHDXFXDXAXLVESQGEXIDRIEYNVEHAVDYVE

SEQ ID NO: 41 - protein, 211 aa, Artificial sequence
  Synthetic construct
MDNLSSEEIQQRAHQITDESLESTRRILGLAIESQDAGIKTITMLDEQKEQLNRIEEGLDQINKDMRETEKTLTELNKCCGLCVCPCNRTKNFESGKAYKTTWGDGGENSPCNVVSKQPGPVTNGQLQQPTTGAASGGYIKRITNDAREDEMEENLTQVGSILGNLKDMALNIGNEIDAQNPQIKRITDKADTNRDRIDIANARAKKLIDS

SEQ ID NO: 42 - protein, 114 aa, Artificial sequence
  Synthetic construct
PPKFKRHLNDDDVTGSVKSERRNLLEDDSDEEEDFFLRGPSGPRFGPRNDKIKHVQNQVDEVIDVMQENITKVIERGERLDELQDKSESLSDNATAFSNRSKQLRRQMWWRGCK

SEQ ID NO: 43 - protein, 69 aa, Artificial sequence
  Synthetic construct
AGKELKQCQQQADEVTEIMLNNFDKVLERHGKLAELEQRSDQLLDMSSAFSKTTKTLAQQKRWENIRCR

SEQ ID NO: 44 - protein, 187 aa, Artificial sequence
  Synthetic construct
AILFAVVARGTTILAKHAWCGGNFLEVTEQILAKIPSENNKLTYSHGNYLFHYICQDRIVYLCITDDDFERSRAFSFLNEVKKRFQTTYGSRAQTALPYAMNSEFSSVLAAQLKHHSENKSLDKVMETQAQVDELKGIMVRNIDLVAQRGERLELLIDKTENLVDSSVTFKTTSRNLARAMCMKNIK

SEQ ID NO: 45 - protein, 74 aa, Artificial sequence
  Synthetic construct
EEASGSAGNDRVRNLQSEVEGVKNIMTQNVERILSRGENLDHLRNKTEDLEATSEHFKTTSQKVARKFWWKNVK

SEQ ID NO: 46 - protein, 45 aa, Artificial sequence
  Synthetic construct
EIIRLENSIRELHDMFMDMAMLVESQGEMIDRIEYNVEHAVDYVE

SEQ ID NO: 47 - protein, 40 aa, Artificial sequence
  Synthetic construct
EIIKLENSIRELHDMFMDMAMLVESQGEMIDRIEYNVEHA

SEQ ID NO: 48 - protein, 45 aa, Artificial sequence
  Synthetic construct
STRRMLQLVEESKDAGIRTLVMLDEQGEQLDRVEEGMNHINQDMK

SEQ ID NO: 49 - protein, 40 aa, Artificial sequence
  Synthetic construct
STRRMLQLVEESKDAGIRTLVMLDEQGEQLDRVEEGMNHI

SEQ ID NO: 50 - protein, 45 aa, Artificial sequence
  Synthetic construct
NLEQVSGIIGNLRHMALDMGNEIDTQNRQIDRIMEKADSNKTRID

SEQ ID NO: 51 - protein, 40 aa, Artificial sequence
  Synthetic construct
NLEQVSGIIGNLRHMALDMGNEIDTQNRQIDRIMEKADSN

SEQ ID NO: 52 - protein, 45 aa, Artificial sequence
  Synthetic construct
RLQQTQAQVDEVVDIMRVNVDKVLERDQKLSELDDRADALQAGAS

SEQ ID NO: 53 - protein, 40 aa, Artificial sequence
  Synthetic construct
RLQQTQAQVDEVVDIMRVNVDKVLERDQKLSELDDRADAL

SEQ ID NO: 54 - protein, 56 aa, Artificial sequence
  Synthetic construct
EIIKLENSIRELHDMFMDMAMLVESQGEMIDRIEYNVEHAXXAGCKNFFWKTFTSC

SEQ ID NO: 55 - protein, 56 aa, Artificial sequence; identical to SEQ ID NO: 26
  Synthetic construct

SEQ ID NO: 56 - protein, 58 aa, Artificial sequence
  Synthetic construct
EIIKLENSIRELHDMFMDMAMLVESQGEMIDRIEYNVEHAVDYVEXXRPKPQQFFGLM

SEQ ID NO: 57 - protein, 58 aa, Artificial sequence
  Synthetic construct
RLQQTQAQVDEVVDIMRVNVDKVLERDQKLSELDDRADALQAGASXXRPKPQQFFGLM

SEQ ID NO: 58 - protein, 58 aa, Artificial sequence
  Synthetic construct
AEIIKLENSIRELHDMFMDMAMLVESQGEMIDRIEYNVEHAVDYVERXXCYFQNCPRG

SEQ ID NO: 59 - protein, 57 aa, Artificial sequence
  Synthetic construct
CYFQNCPRGXXAEIIKLENSIRELHDMFMDMAMLVESQGEMIDRIEYNVEHAVDYVE

SEQ ID NO: 60 - protein, 27 aa, Artificial sequence
  Synthetic construct
EIIRLENSIRELHDMFMDMAMLVESQG

SEQ ID NO: 61 - protein, 31 aa, Artificial sequence
  Synthetic construct
EIIKLENSIRELHDMFMDMAMLVESQGEMID

SEQ ID NO: 62 - protein, 50 aa, Artificial sequence; identical to SEQ ID NO: 7
  Synthetic construct

SEQ ID NO: 63 - protein, 39 aa, Artificial sequence
  Synthetic construct
ERKAKYAKMEAEREVMRQGIRDKYGIKKGSGSGGIKVAV

SEQ ID NO: 64 - protein, 206 aa, Artificial sequence
  Synthetic construct
MAEDADMRNELEEMQRRADQLADESLESTRRMLQLVEESKDAGIRTLVMLDEQGEQLERIEEGMDQINKDMKEAEKNLTDLGKFAGLAVAPANKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 65 - protein, 211 aa, Artificial sequence; identical to SEQ ID NO: 41
  Synthetic construct

SEQ ID NO: 66 - protein, 59 aa, Artificial sequence
  Synthetic construct
EGRHKDIVRLESSIKELHDMFMDIAMLVENQGEMLDNIELNVMHTVDHVEKARDETKRA

SEQ ID NO: 67 - protein, 96 aa, Artificial sequence
  Synthetic construct
MSATAATVPPAAPAGEGGPPAPPPNLTSNRRLQQTQAQVDEVVDIMRVNVDKVLERDQKLSELDDRADALQAGASQFETSAAKLKRKYAAANLKMM

SEQ ID NO: 68 - protein, 60 aa, Artificial sequence
  Synthetic construct
NLTSNRRLQQTQAQVDEVVDIMRVNVDKVLERDQKLSELDDRADALQAGASQFETSAAKL

SEQ ID NO: 69 - protein, 84 aa, Artificial sequence
  Synthetic construct
MSATAATVPPAAPAGEGGPPAPPPNLTSNRRLQQTQAQVDEVVDIMRVNVDKVLERDQKLSELDDRADALQAGASQFETSAAKL

SEQ ID NO: 70 - protein, 449 aa, Artificial sequence
  Synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKA

SEQ ID NO: 71 - protein, 425 aa, Artificial sequence
  Synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNI

SEQ ID NO: 72 - protein, 420 aa, Artificial sequence
  Synthetic construct
SILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 73 - protein, 118 aa, Artificial sequence
  Synthetic construct
GSEGRHKDIVRLESSIKELHDMFMDIAMLVENQGEMLDNIELNVMHTVDHVEKARDEAKRAGNRRLQQTQAQVDEVVDIMRVNVDKVLERDQKLSELDDRADALQAGASQFETSAAKL

SEQ ID NO: 74 - protein, 55 aa, Artificial sequence
  Synthetic construct
RLQQTQAQVDEVVDIMRVNVDKVLERDQKLSELDDRADALQAGASQFETSAAKLK

SEQ ID NO: 75 - protein, 55 aa, Artificial sequence
  Synthetic construct
EIIKLENSIRELHDMFMDMAMLVESQGEMIDRIEYNVEHAVDYVERAVSDTKKAV

SEQ ID NO: 76 - protein, 55 aa, Artificial sequence
  Synthetic construct
STRRMLQLVEESKDAGIRTLVMLDEQGEQLDRVEEGMNHINQDMKEAEKNLKDLG

SEQ ID NO: 77 - protein, 55 aa, Artificial sequence
  Synthetic construct
NLEQVSGIIGNLRHMALDMGNEIDTQNRQIDRIMEKADSNKTRIDEANQRATKML